SEQUENCE LISTING (149 sequences)

SEQ ID NO: 1 - DNA, 20 bp, Artificial Sequence
  Synthetic sequence
gccgcagtctcgtagtccgg

SEQ ID NO: 2 - DNA, 20 bp, Artificial Sequence
  Synthetic sequence
aggccgcagtctcgtagtcc

SEQ ID NO: 3 - DNA, 21 bp, Artificial Sequence
  Synthetic sequence
aggccgcagtctcgtagtccg

SEQ ID NO: 4 - DNA, 20 bp, Artificial Sequence
  Synthetic sequence
cgcagtctcgtagtccggtg

SEQ ID NO: 5 - DNA, 20 bp, Artificial Sequence
  Synthetic sequence
gatatggcccagaaaggccg

SEQ ID NO: 6 - DNA, 11 bp, Artificial Sequence
  Synthetic sequence
cctttctgggc

SEQ ID NO: 7 - DNA, 17 bp, Artificial Sequence
  Synthetic sequence
ggactacgagagcgcgg

SEQ ID NO: 8 - DNA, 18 bp, Homo sapiens
ccgcggtctcgtagtccg

SEQ ID NO: 9 - protein, 6 aa, Homo sapiens
PRSRSP

SEQ ID NO: 10 - DNA, 30 bp, Artificial Sequence
  Synthetic sequence
gaaaggccgcagtctcgtagtccggtgagc

SEQ ID NO: 11 - protein, 10 aa, Artificial Sequence
  Synthetic sequence
ERPQSRSPVS

SEQ ID NO: 12 - DNA, 30 bp, Homo sapiens
gaaaggccgcggtctcgtagtccggtgagc

SEQ ID NO: 13 - protein, 10 aa, Homo sapiens
ERPRSRSPVS

SEQ ID NO: 14 - DNA, 36 bp, Homo sapiens
ccagaaaggccgcggtctcgtagtccggtgagccgg

SEQ ID NO: 15 - DNA, 14 bp, Homo sapiens
aggccgcggtctcg

SEQ ID NO: 16 - DNA, 14 bp, Artificial Sequence
  Synthetic sequence
  misc_feature (8)..(8): n is a, c, g, or t
aggccgcngtctcg

SEQ ID NO: 17 - DNA, 14 bp, Artificial Sequence
  Synthetic sequence
aggccgcagtctcg

SEQ ID NO: 18 - DNA, 20 bp, Homo sapiens
gccgcagtctcgtagtccgg

SEQ ID NO: 19 - DNA, 20 bp, Homo sapiens
gccgccgcctggtagtccgg

SEQ ID NO: 20 - DNA, 20 bp, Homo sapiens
gcagcagtcttgtagtctgg

SEQ ID NO: 21 - DNA, 20 bp, Homo sapiens
actgcagtcttgaagtccgg

SEQ ID NO: 22 - DNA, 20 bp, Homo sapiens
gcatcagtctcttagtcctg

SEQ ID NO: 23 - DNA, 20 bp, Homo sapiens
gccgcggcctggtattccgg

SEQ ID NO: 24 - DNA, 20 bp, Homo sapiens
gcctcagtcccgtggtcctg

SEQ ID NO: 25 - DNA, 20 bp, Homo sapiens
gccccagtctcctagtctga

SEQ ID NO: 26 - DNA, 20 bp, Homo sapiens
gccccagtcttgtggtcctg

SEQ ID NO: 27 - DNA, 22 bp, Homo sapiens
aaaggccgcggtctcgtagtcc

SEQ ID NO: 28 - DNA, 22 bp, Artificial Sequence
  Synthetic sequence
aaaggccgcagtctcgtagtcc

SEQ ID NO: 29 - DNA, 36 bp, Homo sapiens
ccagagcggccacgttctcgaagtccaatgagccga

SEQ ID NO: 30 - DNA, 30 bp, Artificial Sequence
  Synthetic sequence
gagcggccacagtctcgaagtccaatgagc

SEQ ID NO: 31 - protein, 10 aa, Artificial Sequence
  Synthetic sequence
ERPQSRSPMS

SEQ ID NO: 32 - DNA, 24 bp, Mus musculus
gagcggccacgttctcgaagtcca

SEQ ID NO: 33 - DNA, 24 bp, Artificial Sequence
  Synthetic sequence
  misc_feature (11)..(12): n is a, c, g, or t
gagcggccacnntctcgaagtcca

SEQ ID NO: 34 - DNA, 24 bp, Artificial Sequence
  Synthetic sequence
gagcggccacagtctcgaagtcca

SEQ ID NO: 35 - DNA, 30 bp, Artificial Sequence
  Synthetic sequence
gagcggccacggtctcgaagtccaatgagc

SEQ ID NO: 36 - protein, 10 aa, Artificial Sequence
  Synthetic sequence
ERPRSRSPMS

SEQ ID NO: 37 - DNA, 15 bp, Artificial Sequence
  Synthetic sequence
gccacgttctcgaag

SEQ ID NO: 38 - DNA, 15 bp, Artificial Sequence
  Synthetic sequence
gccacagtctcgaag

SEQ ID NO: 39 - DNA, 15 bp, Artificial Sequence
  Synthetic sequence
gccacggtctcgaag

SEQ ID NO: 40 - DNA, 42 bp, Artificial Sequence
  Synthetic sequence
tcacagatatggcccagaaaggccgcggtctagtagtccggt

SEQ ID NO: 41 - DNA, 14 bp, Artificial Sequence
  Synthetic sequence
cggtctcgtagtcc

SEQ ID NO: 42 - DNA, 14 bp, Artificial Sequence
  Synthetic sequence
cggtctagtcgtcc

SEQ ID NO: 43 - DNA, 14 bp, Artificial Sequence
  Synthetic sequence
  misc_feature (3)..(3): n is a, c, g, or t
  misc_feature (7)..(7): n is a, c, g, or t
cgntctngtagtcc

SEQ ID NO: 44 - DNA, 52 bp, Homo sapiens
tcacagatatggcccagaaaggccgcggtctcgtagtccggtgagccggtca

SEQ ID NO: 45 - protein, 15 aa, Homo sapiens
YGPERPRSRSPVSRS

SEQ ID NO: 46 - DNA, 18 bp, Artificial Sequence
  Synthetic sequence
aaggccgcagtctcgtag

SEQ ID NO: 47 - DNA, 18 bp, Artificial Sequence
  Synthetic sequence
aaggccgcggtctcgtag

SEQ ID NO: 48 - DNA, 48 bp, Homo sapiens
aggtatggtccagagcggccacgttctcgaagtccaatgagccgatca

SEQ ID NO: 49 - protein, 15 aa, Homo sapiens
YGPERPRSRSPMSRS

SEQ ID NO: 50 - DNA, 21 bp, Artificial Sequence
  Synthetic sequence
agagcggccacgttctcgaag

SEQ ID NO: 51 - protein, 1227 aa, Homo sapiens
MVLAAAMSQDADPSGPEQPDRVACSVPGARASPAPSGPRGMQQPPPPPQPPPPPQAGLPQIIQNAAKLLDKNPFSVSNPNPLLPSPASLQLAQLQAQLTLHRLKLAQTAVTNNTAAATVLNQVLSKVAMSQPLFNQLRHPSVITGPHGHAGVPQHAAAIPSTRFPSNAIAFSPPSQTRGPGPSMNLPNQPPSAMVMHPFTGVMPQTPGQPAVILGIGKTGPAPATAGFYEYGKASSGQTYGPETDGQPGFLPSSASTSGSVTYEGHYSHTGQDGQAAFSKDFYGPNSQGSHVASGFPAEQAGGLKSEVGPLLQGTNSQWESPHGFSGQSKPDLTAGPMWPPPHNQPYELYDPEEPTSDRTPPSFGGRLNNSKQGFIGAGRRAKEDQALLSVRPLQAHELNDFHGVAPLHLPHICSICDKKVFDLKDWELHVKGKLHAQKCLVFSENAGIRCILGSAEGTLCASPNSTAVYNPAGNEDYASNLGTSYVPIPARSFTQSSPTFPLASVGTTFAQRKGAGRVVHICNLPEGSCTENDVINLGLPFGKVTNYILMKSTNQAFLEMAYTEAAQAMVQYYQEKSAVINGEKLLIRMSKRYKELQLKKPGKAVAAIIQDIHSQRERDMFREADRYGPERPRSRSPVSRSLSPRSHTPSFTSCSSSHSPPGPSRADWGNGRDSWEHSPYARREEERDPAPWRDNGDDKRDRMDPWAHDRKHHPRQLDKAELDERPEGGRPHREKYPRSGSPNLPHSVSSYKSREDGYYRKEPKAKSDKYLKQQQDAPGRSRRKDEARLRESRHPHPDDSGKEDGLGPKVTRAPEGAKAKQNEKNKTKRTDRDQEGADDRKENTMAENEAGKEEQEGMEESPQSVGRQEKEAEFSDPENTRTKKEQDWESESEAEGESWYPTNMEELVTVDEVGEEEDFIVEPDIPELEEIVPIDQKDKICPETCLCVTTTLDLDLAQDFPKEGVKAVGNGAAEISLKSPRELPSASTSCPSDMDVEMPGLNLDAERKPAESETGLSLEDSDCYEKEAKGVESSDVHPAPTVQQMSSPKPAEERARQPSPFVDDCKTRGTPEDGACEGSPLEEKASPPIETDLQNQACQEVLTPENSRYVEMKSLEVRSPEYTEVELKQPLSLPSWEPEDVFSELSIPLGVEFVVPRTGFYCKLCGLFYTSEETAKMSHCRSAVHYRNLQKYLSQLAEEGLKETEGADSPRPEDSGIVPRFERKKL

SEQ ID NO: 52 - protein, 1199 aa, Mus musculus
MVLAVAMSQDADPSGPEQPDRDACVMPGVQGPSVPQGQQGMQPLPPPPPPQPQASLPQIIQNAAKLLDKSPFSVNNQNPLLTSPASVQLAQIQAQLTLHRLKMAQTAVTNNTAAATVLNQVLSKVAMSQPLFNQLRHPSVLGTAHGPTGVSQHAASVPSAHFPSTAIAFSPPSQTGGPGPSVSLPSQPPNAMVVHTFSGVVPQTPAQPAVILSLGKAGPTPATTGFYDYGKANSGQAYGSETEGQPGFLPASASATASGSMTYEGHYSHTGQDGQPAFSKDFYGPNAQGPHIAGGFPADQTGSMKGDVGGLLQGTNSQWERPPGFSGQNKPDITAGPSLWAPPASQPYELYDPEEPTSDRAPPAFGSRLNNSKQGFGCSCRRTKEGQAVLSVRPLQGHQLNDFRGLAPLHLPHICSICDKKVFDLKDWELHVKGKLHAQKCLLFSESAGLRSIRASGEGTLSASANSTAVYNPTGNEDYTSNLGTSYAAIPTRAFAQSNPVFPSASSGTSFAAQRKGAGRVVHICNLPEGSCTENDVINLGLPFGKVTNYILMKSTNQAFLEMAYTEAAQAMVQYYQEKPAIINGEKLLIRMSTRYKELQLKKPGKNVAAIIQDIHSQRERDMLREADRYGPERPRSRSPMSRSLSPRSHSPPGPSRADWGNGRDSYAWRDEDRETVPRRENGEDKRDRLDVWAHDRKHYPRQLDKAELDERLEGGRGYREKYLKSGSPGPLHSVSGYKGREDGYHRKEPKAKLDKYPKQQPDVPGRSRRKEEARLREPRHPHPEDSGKAEDLEPKITRAPDGTKSKQSEKSKTKRADRDQEGADDKKESQLAENEAGAEEQEGMVGIQQEGTESCDPENTRTKKGQDCDSGSEPEGDNWYPTNMEELVTVDEVGEEDFIMEPDLPELEEIVPIDQKDKTLPKICTCVTATLGLDLAKDFTKQGETLGNGDAELSLKLPGQVPSTSASCPNDTDLEMPGLNLDAERKPAESETGLSLEVSNCYEKEARGEEDSDVSLAPAVQQMSSPQPADERARQSSPFLDDCKARGSPEDGSHEASPLEGKASPPTESDLQSQACRENPRYMEVKSLNVRSPEFTEAELKEPLSLPSWEPEVFSELSIPLGVEFVVPRTGFYCKLCGLFYTSEEAAKVSHCRSTVHYRNLQKYLSQLAEEGLKETEGTDSPSPERGGIGPHLERKKL

SEQ ID NO: 53 - DNA, 86 bp, Artificial Sequence
  Synthetic sequence
gtttaagtactctgtgctggaaacagcacagaatctacttaaacaaggcaaaatgccgtgtttatctcgtcaacttgttggcgaga

SEQ ID NO: 54 - protein, 18 aa, Artificial Sequence
  Synthetic sequence
EGRGSLLTCGDVEENPGP

SEQ ID NO: 55 - protein, 20 aa, Artificial Sequence
  Synthetic sequence
QCTNYALLKLAGDVESNPGP

SEQ ID NO: 56 - protein, 19 aa, Artificial Sequence
  Synthetic sequence
ATNFSLLKQAGDVEENPGP

SEQ ID NO: 57 - protein, 23 aa, Artificial Sequence
  Synthetic sequence
PVKQLLNFDLLKLAGDVESNPGP

SEQ ID NO: 58 - protein, 41 aa, Artificial Sequence
  Synthetic sequence
RMRKFKNKGKDTAELRRRRVEVSVELRKAKKDEQILKRRNV

SEQ ID NO: 59 - protein, 8 aa, Artificial Sequence
  Synthetic sequence
VSRKRPRP

SEQ ID NO: 60 - protein, 8 aa, Artificial Sequence
  Synthetic sequence
PPKKARED

SEQ ID NO: 61 - protein, 8 aa, Artificial Sequence
  Synthetic sequence
PQPKKKPL

SEQ ID NO: 62 - protein, 12 aa, Artificial Sequence
  Synthetic sequence
SALIKKKKKMAP

SEQ ID NO: 63 - protein, 5 aa, Artificial Sequence
  Synthetic sequence
DRLRR

SEQ ID NO: 64 - protein, 7 aa, Artificial Sequence
  Synthetic sequence
PKQKKRK

SEQ ID NO: 65 - protein, 10 aa, Artificial Sequence
  Synthetic sequence
RKLKKKIKKL

SEQ ID NO: 66 - protein, 10 aa, Artificial Sequence
  Synthetic sequence
REKKKFLKRR

SEQ ID NO: 67 - protein, 20 aa, Artificial Sequence
  Synthetic sequence
KRKGDEVDGVDEVAKKKSKK

SEQ ID NO: 68 - protein, 17 aa, Artificial Sequence
  Synthetic sequence
RKCLQAGMNLEARKTKK

SEQ ID NO: 69 - DNA, 4101 bp, Stapholococcus pyogenes
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 70 - DNA, 115 bp, Artificial Sequence
  Synthetic sequence
ggccactccctctctgcgcgctcgctcgctcactgaggccgggcgaccaaaggtcgcccgacgcccgggctttgcccgggcggcctcagtgagcgagcgagcgcgcagagaggga

SEQ ID NO: 71 - DNA, 141 bp, Artificial Sequence
  Synthetic sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 72 - DNA, 141 bp, Artificial Sequence
  Synthetic sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 73 - DNA, 60 bp, Artificial Sequence
  Synthetic sequence
tagcaataaaggatcgtttattttcattggaagcgtgtgttggttttttgatcaggcgcg

SEQ ID NO: 74 - DNA, 48 bp, Artificial Sequence
  Synthetic sequence
aagcgtcctgctgctactaagaaagctggtcaagctaagaaaaagaaa

SEQ ID NO: 75 - DNA, 51 bp, Artificial Sequence
  Synthetic sequence
atggccccaaagaagaagcggaaggtcggtatccacggagtcccagcagcc

SEQ ID NO: 76 - DNA, 166 bp, Artificial Sequence
  Synthetic sequence
tgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggtt

SEQ ID NO: 77 - DNA, 225 bp, Artificial Sequence
  Synthetic sequence
gaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccaca

SEQ ID NO: 78 - DNA, 1155 bp, Artificial Sequence
  Synthetic sequence
catgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccg

SEQ ID NO: 79 - DNA, 25 bp, Artificial Sequence
  Synthetic sequence
caccgctcaccggactacgagaccg

SEQ ID NO: 80 - DNA, 25 bp, Artificial Sequence
  Synthetic sequence
aaaccggtctcgtagtccggtgagc

SEQ ID NO: 81 - DNA, 19 bp, Artificial Sequence
  Synthetic sequence
gccagtgctgtgcttagga

SEQ ID NO: 82 - DNA, 20 bp, Artificial Sequence
  Synthetic sequence
tggtgtttgcgatcatgtgc

SEQ ID NO: 83 - DNA, 127 bp, Artificial Sequence
  Synthetic sequence
gtctgtgtgtgggtggggtgggatgggaggtgtgaagattctaaatcctgctccttggctccctcacagatatggcccagaaaggccgcagtctcgtagtccggtgagccggtcactctccccgagg

SEQ ID NO: 84 - DNA, 24 bp, Artificial Sequence
  Synthetic sequence
caccgccgcagtctcgtagtccgg

SEQ ID NO: 85 - DNA, 24 bp, Artificial Sequence
  Synthetic sequence
aaacccggactacgagactgcggc

SEQ ID NO: 86 - DNA, 25 bp, Artificial Sequence
  Synthetic sequence
caccgaggccgcagtctcgtagtcc

SEQ ID NO: 87 - DNA, 25 bp, Artificial Sequence
  Synthetic sequence
aaacggactacgagactgcggcctc

SEQ ID NO: 88 - DNA, 26 bp, Artificial Sequence
  Synthetic sequence
caccgaggccgcagtctcgtagtccg

SEQ ID NO: 89 - DNA, 26 bp, Artificial Sequence
  Synthetic sequence
agaccggactacgagactgcggcctc

SEQ ID NO: 90 - DNA, 25 bp, Artificial Sequence
  Synthetic sequence
caccgcgcagtctcgtagtccggtg

SEQ ID NO: 91 - DNA, 25 bp, Artificial Sequence
  Synthetic sequence
aaaccaccggactacgagactgcgc

SEQ ID NO: 92 - DNA, 21 bp, Artificial Sequence
  Synthetic sequence
accgcagctaggaataatgga

SEQ ID NO: 93 - DNA, 19 bp, Artificial Sequence
  Synthetic sequence
gcctcagttccgaaaacca

SEQ ID NO: 94 - DNA, 21 bp, Artificial Sequence
  Synthetic sequence
ccaatgagtatggcagtgtca

SEQ ID NO: 95 - DNA, 21 bp, Artificial Sequence
  Synthetic sequence
tacgttccggaagtaatttgc

SEQ ID NO: 96 - DNA, 20 bp, Artificial Sequence
  Synthetic sequence
tttgttgagggcagagccaa

SEQ ID NO: 97 - DNA, 20 bp, Artificial Sequence
  Synthetic sequence
tggcactctttgcttggtga

SEQ ID NO: 98 - DNA, 27 bp, Artificial Sequence
  Synthetic sequence
gactctctgtgggccttcaaagatgga

SEQ ID NO: 99 - DNA, 25 bp, Artificial Sequence
  Synthetic sequence
tattggcgctcgtctgcccaatctc

SEQ ID NO: 100 - DNA, 22 bp, Artificial Sequence
  Synthetic sequence
ctttcctgactacttccctggt

SEQ ID NO: 101 - DNA, 21 bp, Artificial Sequence
  Synthetic sequence
gagtctggcagtggaacaaga

SEQ ID NO: 102 - DNA, 26 bp, Artificial Sequence
  Synthetic sequence
gcaacttggcaaagggaagaaaaaca

SEQ ID NO: 103 - DNA, 27 bp, Artificial Sequence
  Synthetic sequence
cagagtaccactgcctacccactacaa

SEQ ID NO: 104 - DNA, 20 bp, Artificial Sequence
  Synthetic sequence
gcatgtgtctccgggttcaa

SEQ ID NO: 105 - DNA, 20 bp, Artificial Sequence
  Synthetic sequence
gcggctttcccactgaaatc

SEQ ID NO: 106 - DNA, 25 bp, Artificial Sequence
  Synthetic sequence
tgaactggacccccgaggtgtagcc

SEQ ID NO: 107 - DNA, 25 bp, Artificial Sequence
  Synthetic sequence
tttcctaagagtcggtcggcttgag

SEQ ID NO: 108 - DNA, 20 bp, Artificial Sequence
  Synthetic sequence
atcctttgggtccaaccagc

SEQ ID NO: 109 - DNA, 21 bp, Artificial Sequence
  Synthetic sequence
agagcacttggatagttggct

SEQ ID NO: 110 - DNA, 25 bp, Artificial Sequence
  Synthetic sequence
gcccccttaggtgtgcccgtatctt

SEQ ID NO: 111 - DNA, 25 bp, Artificial Sequence
  Synthetic sequence
gcagtgaggctgtgtagtcttgggc

SEQ ID NO: 112 - DNA, 24 bp, Artificial Sequence
  Synthetic sequence
caccgaccggctcaccggactacg

SEQ ID NO: 113 - DNA, 24 bp, Artificial Sequence
  Synthetic sequence
aaaccgtagtccggtgagccggtc

SEQ ID NO: 114 - DNA, 127 bp, Artificial Sequence
  Synthetic sequence
gtgtgggtggggtgggatgggaggtgtgaagattctaaatcctgctccttggctccctcacagatatggcccagaaaggccgcggtctagtagtccggtgagccggtcactctccccgaggtcccac

SEQ ID NO: 115 - DNA, 29 bp, Artificial Sequence
  Synthetic sequence
caccgatatggcccagaaaggccggtttt

SEQ ID NO: 116 - DNA, 29 bp, Artificial Sequence
  Synthetic sequence
ctctaaaaccggcctttctgggccatatc

SEQ ID NO: 117 - DNA, 67 bp, Artificial Sequence
  Synthetic sequence
agagctagaaatagcaagttaaaataaggctagtccgttatcaacttgaaaaagtggcaccgagtcg

SEQ ID NO: 118 - DNA, 67 bp, Artificial Sequence
  Synthetic sequence
gcaccgactcggtgccactttttcaagttgataacggactagccttattttaacttgctatttctag

SEQ ID NO: 119 - DNA, 32 bp, Artificial Sequence
  Synthetic sequence
gtgcggactacgagagcgcggcctttctgggc

SEQ ID NO: 120 - DNA, 32 bp, Artificial Sequence
  Synthetic sequence
aaaagcccagaaaggccgcgctctcgtagtcc

SEQ ID NO: 121 - DNA, 25 bp, Artificial Sequence
  Synthetic sequence
caccgctcaccggactacgagagcg

SEQ ID NO: 122 - DNA, 25 bp, Artificial Sequence
  Synthetic sequence
aaaccgctctcgtagtccggtgagc

SEQ ID NO: 123 - DNA, 74 bp, Artificial Sequence
  Synthetic sequence
gtgcggactacgagagcgcggcctttctgggcttgacgcggttctatctagttacgcgttaaaccaactagaaa

SEQ ID NO: 124 - DNA, 74 bp, Artificial Sequence
  Synthetic sequence
aaaatttctagttggtttaacgcgtaactagatagaaccgcgtcaagcccagaaaggccgcgctctcgtagtcc

SEQ ID NO: 125 - DNA, 42 bp, Artificial Sequence
  Synthetic sequence
tttttttcaggttggaccggtgccaccatgaaacggacagcc

SEQ ID NO: 126 - DNA, 21 bp, Artificial Sequence
  Synthetic sequence
gcccacagagttggtgccgat

SEQ ID NO: 127 - DNA, 20 bp, Artificial Sequence
  Synthetic sequence
tcggcaccaactctgtgggc

SEQ ID NO: 128 - DNA, 21 bp, Artificial Sequence
  Synthetic sequence
gctgtttcccctggccagagg

SEQ ID NO: 129 - DNA, 43 bp, Artificial Sequence
  Synthetic sequence
tttttttcaggttggaccggtgccaccatgaaacggacagccg

SEQ ID NO: 130 - DNA, 24 bp, Artificial Sequence
  Synthetic sequence
ttgatgctctggatgaagctccgc

SEQ ID NO: 131 - DNA, 55 bp, Artificial Sequence
  Synthetic sequence
agaagaaatataagactcgagttaattaagaggtcgggataaaagcagtctgggc

SEQ ID NO: 132 - DNA, 39 bp, Artificial Sequence
  Synthetic sequence
ccgtttcatggtggcaccggttcccacggagcggtgggt

SEQ ID NO: 133 - DNA, 43 bp, Artificial Sequence
  Synthetic sequence
ggtgggctctatgggcggccgagggcctatttcccatgattcc

SEQ ID NO: 134 - DNA, 51 bp, Artificial Sequence
  Synthetic sequence
gctggcgcgcctttttctagagcggccgcaaaaaaagcaccgactcggtgc

SEQ ID NO: 135 - DNA, 43 bp, Artificial Sequence
  Synthetic sequence
cggtgggctctatggctcgagagggcctatttcccatgattcc

SEQ ID NO: 136 - DNA, 25 bp, Artificial Sequence
  Synthetic sequence
caccgctcattggacttcgagaacg

SEQ ID NO: 137 - DNA, 25 bp, Artificial Sequence
  Synthetic sequence
aaaccgttctcgaagtccaatgagc

SEQ ID NO: 138 - DNA, 26 bp, Artificial Sequence
  Synthetic sequence
ggtagagggcagagagtgtcttaggg

SEQ ID NO: 139 - DNA, 26 bp, Artificial Sequence
  Synthetic sequence
ccttcgagtcgctcatccaactcagc

SEQ ID NO: 140 - DNA, 127 bp, Artificial Sequence
  Synthetic sequence
gtctccatctgggtgatgcaggttacgagctctgcagagtctaaaccctgtctcttcccttcctcccaggtatggtccagagcggccacagtctcgaagtccaatgagccgatcactctccccaaga

SEQ ID NO: 141 - DNA, 24 bp, Artificial Sequence
  Synthetic sequence
caccgccacagtctcgaagtccaa

SEQ ID NO: 142 - DNA, 24 bp, Artificial Sequence
  Synthetic sequence
aaacttggacttcgagactgtggc

SEQ ID NO: 143 - DNA, 20 bp, Artificial Sequence
  Synthetic sequence
atggcttacacagaagccgc

SEQ ID NO: 144 - DNA, 20 bp, Artificial Sequence
  Synthetic sequence
ccttcgagtcgctcatccaa

SEQ ID NO: 145 - DNA, 20 bp, Artificial Sequence
  Synthetic sequence
gtaacccgttgaaccccatt

SEQ ID NO: 146 - DNA, 20 bp, Artificial Sequence
  Synthetic sequence
ccatccaatcggtagtagcg

SEQ ID NO: 147 - DNA, 20 bp, Artificial Sequence
  Synthetic sequence
ggagtacacctgcaaagcct

SEQ ID NO: 148 - DNA, 20 bp, Artificial Sequence
  Synthetic sequence
tgcggcttaggttcaggaag

SEQ ID NO: 149 - DNA, 20 bp, Artificial Sequence
  Synthetic sequence
ccttgggcctggagagaaag

(19) United States
(12) Patent Application Publication
(10) Pub. No.: US 2024/0325454 A1
(43) Pub. Date: Oct. 3, 2024

(54) GENOMIC EDITING OF RBM20 MUTATIONS

(71) Applicant: The Board of Regents of The University of Texas System, Austin, TX (US)

(72) Inventors: Eric N. OLSON, Dallas, TX (US); Rhonda BASSEL-DUBY, Dallas, TX (US); Takahiko NISHIYAMA, Dallas, TX (US)

(73) Assignee: The Board of Regents of The University of Texas System, Austin, TX (US)

(21) Appl. No.: 18/575,499

(22) PCT Filed: Jun. 30, 2022
(86) PCT No.: PCT/US2022/035666

§ 371 Date: Dec. 29, 2023

Related U.S. Application Data
(60) Provisional application No. 63/335,647, filed on Apr. 27, 2022.
(60) Provisional application No. 63/218,221, filed on Jul. 2, 2021.

(51) Int. Cl. A61K 35/34; A61K 48/00; C12N 9/22; C12N 15/11; C12N 15/86

(52) U.S. Cl. CPC A61K 35/34; A61K 48/005; C12N 9/22; C12N 15/11; C12N 15/86; C12N 2310/20; C12N 2750/14143

(57) ABSTRACT

Disclosures herein are directed to compositions comprising single guide RNA (sgRNA) designed for a CRISPR/Cas9 system and method of using thereof for preventing, ameliorating or treating one or more cardiomyopathies. For example, provided herein are composition and methods for the correction of dilated cardiomyopathy by precise genomic editing of RBM20 mutations in human cells and mice.

### REFERENCE TO RELATED APPLICATIONS

The present application claims the priority benefit of U.S. provisional application No. 63/335,647, filed Apr. 27, 2022, and U.S. provisional application No. 63/218,221, filed Jul. 2, 2021, the entire contents of each of which are incorporated herein by reference.

### BACKGROUND

### 1. Field

The present invention relates generally to the fields of molecular biology, medicine, and genetics. More particularly, it concerns compositions and uses thereof for genome editing to correct mutations in vivo using a nucleotide editing approach.

### 2. Description of Related Art

Cardiomyopathy is a disease of the heart muscle that causes the heart muscle to become enlarged, thick, and/or rigid. As cardiomyopathy progresses, the heart becomes weaker and can lead to heart failure or irregular heartbeats (i.e., arrhythmias). Dilated cardiomyopathy (DCM) is a severe myocardial disease characterized by left ventricular or biventricular enlargement and systolic dysfunction, representing a major risk factor of heart failure (1). Mutations in RNA binding motif protein 20 (RBM20) are a common cause of cardiomyopathy and account for 2-5% of familial DCM patients (2, 3). Many RBM20 mutations cluster within the arginine/serine rich (RS-rich) domain, which mediates nuclear localization (4-7). Patients with these RBM20 mutations experience a high rate of arrhythmic events and sudden cardiac death (8-10).

RBM20 regulates alternative splicing of many important cardiac genes encoding sarcomere and calcium-regulatory proteins (7, 11). It was postulated that the primary cause of RBM20-associated cardiomyopathy is the lack of alternative splicing of cardiac genes, such as titin (TTN) (12). However, a recent report showed that the RBM20^{R636S} mutation in the RS-rich region induced RBM20 mis-localization and the formation of phase-separated ribonucleoprotein (RNP) granules in the cytoplasm (13), implicating aberrant RNP granules in cardiomyocytes (CM) as a potential cause of DCM (13-16). There are no effective treatments for DCM since most therapies address the symptoms or target secondary effects, while the genetic mutations underlying the disease remain unchanged.

### SUMMARY

Precise CRISPR-Cas9 gene editing technologies, such as base editing (BE) and prime editing (PE), offer the potential to permanently correct disease-causing mutations, and provide new therapeutic approaches to treat cardiomyopathy (17, 18). To this end, the inventors designed sgRNA to correct the RBM20 (R634Q) mutation by CRISPR-Cas9 adenine base editing (ABE), in human induced pluripotent stem cell-derived cardiomyocytes, and observed recovery of functional cardiomyocytes. Additionally, the inventors created a Rbm20 (R636Q) mouse model, which showed severe cardiac dysfunction and sudden death, recapitulating the phenotype observed in DCM patients. The ABE gene editing components were delivered to the mice using an adeno-associated virus (AAV) delivery system to rescue RBM20 cardiomyopathy in vivo. The inventors also designed pegRNA for prime editing (PE) to correct other RBM20 mutations that are not correctable by ABE. These findings provide a promising therapeutic approach for permanent correction of RBM20 mutations and other genetic mutations underlying DCM.

The present disclosure is based, at least in part, on the discovery of guide RNAs (gRNAs) for use with Clustered Regularly Interspaced Short Palindromic Repeats (CRISPR)/CRISPR associate protein 9 (Cas9) systems that successfully reverse phenotypes associated with familial cardiomyopathies, such as DCM, by correcting genetic mutations through base-pair editing. In other words, the present disclosure is directed to compositions comprising single guide RNA (sgRNA) designed for a CRISPR/Cas9 system and method of using thereof for preventing, ameliorating or treating one or more cardiomyopathies.

Aspects of the present disclosure provide gRNAs designed for CRISPR/CAS9 systems for preventing, ameliorating or treating one or more cardiomyopathies. In some embodiments, gRNAs herein may prevent, ameliorate or treat one or more cardiomyopathies comprising dilated cardiomyopathy (DCM).

In some embodiments, gRNAs herein may comprise a polynucleotide sequence having at least 85%, at least 90%, or at least 95% sequence identity with the nucleotide sequence of any one of SEQ ID NOs: 1-4. In some embodiments, gRNAs herein may further comprise a protospacer adjacent motif (PAM).

In one embodiment, provided herein are guide RNAs (gRNAs) comprising a targeting nucleic acid sequence selected from any one of SEQ ID NOs: 1-4. The targeting nucleic acid sequence may have a sequence that is at least 85% identical, 90% identical, or 95% identical to any one of SEQ ID Nos: 1-4. The gRNA may be a single-molecule guide RNA (sgRNA). The gRNA may be for modifying a sequence in the human RBM20 gene.

In some embodiments, gRNAs herein may correct at least one mutation in at least one gene, wherein the at least one gene comprises RBM20. In some embodiments, gRNAs herein may correct a R634Q mutation in a RBM20 gene or its mammalian equivalent thereof.

Other aspects of the present disclosure provide CRISPR/CAS9 systems. In some embodiments, CRISPR/CAS9 systems herein may comprise at least one vector comprising a polynucleotide molecule encoding Cas9 nuclease and at least one gRNA as disclosed herein. In some embodiments, CRISPR/Cas9 systems herein may comprise a Cas9 nuclease comprised from Streptococcus, Staphylococcus, and/or variants thereof.

In one embodiment, provided herein are compositions comprising a gRNA that targets a mutation in human RBM20 and a base editor. The gRNA may comprise a targeting nucleic acid sequence selected from any one of SEQ ID NOs: 1-4. The targeting nucleic acid sequence may have a sequence that is at least 85% identical, 90% identical, or 95% identical to any one of SEQ ID Nos: 1-4. The gRNA may be a single-molecule guide RNA (sgRNA).

The base editor may be an adenine base editor (ABE). The base editor may comprise a CRISPR/Cas nuclease linked to an adenosine deaminase. The CRISPR/Cas nuclease may be catalytically impaired. The CRISPR/Cas nuclease may be a Cas9 nuclease, which may be isolated or derived from Streptococcus pyogenes (e.g., spCas9).

In one embodiment, provided herein are nucleic acids comprising: a sequence encoding a first gRNA that targets mutation in human RBM20, a sequence encoding a base editor, a sequence encoding a first promoter, wherein the first promoter drives expression of the sequence encoding the first gRNA, and a sequence encoding a second promoter, wherein the second promoter drives expression of the sequence encoding the base editor.

The gRNA may comprise a targeting nucleic acid sequence selected from any one of SEQ ID NOs: 1-4. The targeting nucleic acid sequence may have a sequence that is at least 85% identical, 90% identical, or 95% identical to any one of SEQ ID Nos: 1-4. The gRNA may be a single-molecule guide RNA (sgRNA). The base editor may be an adenine base editor (ABE). The base editor may comprise a CRISPR/Cas nuclease linked to an adenosine deaminase. The CRISPR/Cas nuclease may be catalytically impaired. The CRISPR/Cas nuclease may be a Cas9 nuclease, which may be isolated or derived from Streptococcus pyogenes (e.g., spCas9), Staphylococcus aureus (e.g., SaCas9), Staphylococcus auricularis (e.g., SauCas9), or Staphylococcus lugdunensis (e.g., SlugCas9).

The first promoter and/or the second promoter may be a cell-type specific promoter. The cell-type specific promoter may be a cardiomyocyte-specific promoter, such as, for example, a cardiac troponin T (cTnT) promoter. The first promoter may be a U6 promoter, an H1 promoter, or a 7SK promoter.

The nucleic acid may be a DNA or an RNA. The nucleic acid may comprise a polyadenosine (polyA) sequence, which may be a mini polyA sequence. The nucleic acid may be comprised in a composition, which may be comprised in a cell. The nucleic acid may be comprised in a cell, which may be comprised in a composition.

The nucleic acid may be comprised in a vector. The vector may comprise a sequence encoding an inverted terminal repeat (ITR) of a transposable element, such as, for example, a transposon (e.g., a Tn7 transposon). The vector may comprise a sequence encoding a 5′ ITR of a T7 transposon and a sequence encoding a 3′ ITR of a T7 transposon. The vector may be a non-viral vector, such as, for example, a plasmid. The vector may be a viral vector, such as, for example, an adeno-associated viral (AAV) vector or an adenoviral vector. The AAV vector may be replication-defective or conditionally replication defective. The AAV vector may be a recombinant AAV vector. The AAV vector may comprise a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11), AAV9-rh74-HB-P1, AAV9-AAA-P1-SG, AAVrh10, AAVrh74, AAV9P, MyoAAV1A, MyoAAV2A, MyoAAV3A, MyoAAV4A, MyoAAV4C, or MyoAAV4E, or any combination thereof, wherein the number following AAV indicates the AAV serotype. See, e.g., WO2019193119; WO2022053630; Weinmann et al., 2020, Nature Communications, 11:5432; and Tabebordbar et al., 2021, Cell, 184:1-20, each of which is incorporated by reference herein in its entirety.

The vector may be optimized for expression in mammalian cells, such as, for example, human cells.

The vector may be comprised in a composition, which may further comprise a pharmaceutically acceptable carrier. The vector may be comprised in a cell, such as a human cell, a cardiomyocyte, or an induced pluripotent stem (iPS) cell. The cell may be comprised in a composition.

In one embodiment, provided herein are methods for correcting a mutation in human RBM20, the methods comprising contacting a cell with a nucleic acid or vector composition of any one of the present embodiments under conditions suitable for expression of the first gRNA and the adenine base editor, wherein the first gRNA forms a complex with the adenine base editor, wherein the complex modifies a dystrophin splice site thereby restoring correct the coding sequence of RBM20. A cell produced by such a method is also provided.

Other aspects of the present disclosure provide methods of modifying at least one cardiomyopathy-related gene in a cell. In some embodiments, methods of modifying at least one cardiomyopathy-related gene in a cell may comprise contacting the cell with at least one type of vector encoding a CRISPR/CAS9 system herein, wherein the CRISPR/CAS9 system may be directed to a mutant allele of the cardiomyopathy-related gene and/or the at least one type of vector may comprise a polynucleotide molecule encoding Cas9 nuclease and one or more gRNAs as disclosed herein.

Other aspects of the present disclosure provide methods of preventing, ameliorating or treating one or more cardiomyopathies in a subject. In some embodiments, methods of preventing, ameliorating or treating one or more cardiomyopathies in a subject may comprise administering one or more adeno-associated virus (AAV) particles to a subject, wherein the AAV particle may comprise one or more polynucleotides encoding Cas9 nuclease as disclosed herein and one or more gRNAs as disclosed herein.

In one embodiment, provided herein are guide RNAs (gRNAs) comprising a targeting nucleic acid sequence of 5′-GATATGGCCCAGAAAGGCCG-3′ (SEQ ID NO: 5). The targeting nucleic acid sequence may have a sequence that is at least 85% identical, 90% identical, or 95% identical to SEQ ID NO: 5. The gRNA may be a prime editing (pe) gRNA (pegRNA). The gRNA may be for modifying the human RBM20 gene to correct a C1906A mutation.

The gRNA may further comprise a primer binding site comprising a nucleic acid sequence of 5′-CCTTTCTGGGC-3′ (SEQ ID NO: 6). The primer binding site sequence may have a sequence that is at least 85% identical, 90% identical, or 95% identical to SEQ ID NO: 6. The gRNA may further comprise a reverse transcriptase template comprising a nucleic acid sequence of 5′-GGACTACGAGAGCGCGG-3′ (SEQ ID NO: 7). The reverse transcriptase template sequence may have a sequence that is at least 85% identical, 90% identical, or 95% identical to SEQ ID NO: 7.

In one embodiment, provided herein are compositions comprising a gRNA that correct a C1906A mutation the human RBM20 gene and a prime editor. The gRNA may modify the human RBM20 gene to restore the coding sequence of RBM20. The gRNA may comprise a targeting nucleic acid sequence of 5′-GATATGGCCCAGAAAGGCCG-3′ (SEQ ID NO: 5). The targeting nucleic acid sequence may have a sequence that is at least 85% identical, 90% identical, or 95% identical to SEQ ID NO: 5. The gRNA may be a prime editing (pe) gRNA (pegRNA).

The prime editor may comprise a CRISPR/Cas nuclease linked to a reverse transcriptase. The CRISPR/Cas nuclease may be catalytically impaired. The CRISPR/Cas nuclease may be a Cas9 nuclease, which may be isolated or derived from Streptococcus pyogenes (e.g., spCas9). The composition may further comprise a second-strand nicking sgRNA.

In one embodiment, provided herein are nucleic acids comprising: a sequence encoding a first gRNA that targets the human RBM20 gene, a sequence encoding a prime editor, a sequence encoding a first promoter, wherein the first promoter drives expression of the sequence encoding the first gRNA, and a sequence encoding a second promoter, wherein the second promoter drives expression of the sequence encoding the prime editor.

The prime editor may comprise a CRISPR/Cas nuclease linked to a reverse transcriptase. The CRISPR/Cas nuclease may be catalytically impaired. The CRISPR/Cas nuclease may be a Cas9 nuclease, which may be isolated or derived from Streptococcus pyogenes (e.g., spCas9). The composition may further comprise a second-strand nicking sgRNA.

The first promoter and/or the second promoter may be a cell-type specific promoter. The cell-type specific promoter may be a cardiomyocyte-specific promoter, such as, for example, a cardiac troponin T (cTnT) promoter. The first promoter may be a U6 promoter, an H1 promoter, or a 7SK promoter.

The nucleic acid may be a DNA or an RNA. The nucleic acid may comprise a polyadenosine (polyA) sequence, which may be a mini polyA sequence. The nucleic acid may be comprised in a composition, which may be comprised in a cell. The nucleic acid may be comprised in a cell, which may be comprised in a composition.

The nucleic acid may be comprised in a vector. The vector may comprise a sequence encoding an inverted terminal repeat (ITR) of a transposable element, such as, for example, a transposon (e.g., a Tn7 transposon). The vector may comprise a sequence encoding a 5′ ITR of a T7 transposon and a sequence encoding a 3′ ITR of a T7 transposon. The vector may be a non-viral vector, such as, for example, a plasmid. The vector may be a viral vector, such as, for example, an adeno-associated viral (AAV) vector or an adenoviral vector. The AAV vector may be replication-defective or conditionally replication defective. The AAV vector may be a recombinant AAV vector. The AAV vector may comprise a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11) or any combination thereof. The vector may be optimized for expression in mammalian cells, such as, for example, human cells.

The vector may be comprised in a composition, which may further comprise a pharmaceutically acceptable carrier. The vector may be comprised in a cell, such as a human cell, a cardiomyocyte, or an induced pluripotent stem (iPS) cell. The cell may be comprised in a composition.

In one embodiment, provided herein are methods for correcting a mutation in human RBM20, the methods comprising contacting a cell with a nucleic acid or vector composition of any one of the present embodiments under conditions suitable for expression of the first gRNA and the prime editor, wherein the first gRNA forms a complex with the prime editor, wherein the complex modifies a mutation thereby restoring correct the coding sequence of RBM20. A cell produced by such a method is also provided.

In one embodiment, provided herein are methods of treating dilated cardiomyopathy in a subject in need thereof, the methods comprising administering to the subject a therapeutically effective amount of a pharmaceutical composition of any one of the present embodiments. Use of a therapeutically effective amount of a pharmaceutical composition of any one of the present embodiments for treating dilated cardiomyopathy in a subject in need thereof is also provided.

The composition may be administered locally. The composition may be administered directly to a cardiac tissue. The composition may be administered by an intramuscular infusion or injection. The composition may be administered systemically. The composition may be administered by an intravenous infusion or injection.

Following administration of the composition, the subject may exhibit normal architecture of sarcomeric structures, nuclear localization of RBM20, absence of RNP granule formation, or a combination thereof. Following administration of the composition, the subject may exhibit improved LV function.

The subject may be a neonate, an infant, a child, a young adult, or an adult. The subject may be male or female.

Other aspects of the present disclosure provide kits for use in practicing the methods disclosed herein and/or generating any of the constructs disclosed herein. In some embodiments, kits herein may comprise (a) at least one vector comprising a polynucleotide molecule encoding at least one Cas9 nuclease as disclosed herein and one or more gRNAs as disclosed herein, and at least (b) a container.

Other aspects of the present disclosure provide pharmaceutical compositions for use herein. In some embodiments, any of the compositions, vectors, AAV particles, CRISPR/CAS9 system, and/or gRNAs can be formulated as a pharmaceutical composition as disclosed herein. In some embodiments, pharmaceutical compositions herein may comprise at least one vector comprising a polynucleotide molecule encoding Cas9 nuclease herein and one or more gRNAs as disclosed herein and at least one pharmaceutically acceptable carrier, diluent and/or excipient. In some embodiments, pharmaceutical compositions herein may comprise at least one an adeno-associated virus (AAV) vector. In some embodiments, pharmaceutical compositions herein may comprise at least one AAV vector packaged into virus particles. In some embodiments, pharmaceutical compositions herein may comprise AAV particles.

In one embodiment, provided herein are genetically modified mice whose genomes comprise at least one allele of a Rbm20 gene encoded an R636Q mutation. The mouse may have a C57/BL6 genetic background. The genome may be homozygous for alleles of Rbm20 encoded an R636Q mutation. The mice may suffer from cardiac dysfunction. For example, the left ventricular internal dimensions during end-diastole (LVIDd) and end-systole (LVIDs) may be increased. The cardiac dysfunction may be atrial and ventricular dilation. The cardiac dysfunction may be reduced fractional shortening. Also provided is a cell isolated from such a mouse. The cle1 may be a cardiomyocyte.

In one embodiment, provided herein are methods for screening at least one candidate agent in a mouse according to any one of the present embodiments, comprising administering one or more candidate agent to the mouse. The at least one candidate agent may be screened for its ability to improve left ventricular function. The at least one candidate agent may be screened for its ability to rescue cardiac chamber size. The at least one candidate agent may be screened for its ability to increase life span. The candidate agent may comprise a gRNA of any one of the present embodiments.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E. Precise correction of the RBM20^{R634Q} mutation by adenine base editing in iPSCs. (FIG. 1A) Exons 8-10 of the human RBM20 gene highlighting hotspot mutations in exon 9 which encodes the arginine/serine rich (RS-rich) region. The nucleotide sequence shown is SEQ ID NO: 8. The amino acid sequence shown is SEQ ID NO: 9. (FIG. 1B) Illustration depicting adenine base editing (ABE) correction of R634Q mutation using sgRNA1 and ABEmax-VRQR-SpCas9. On-target site is positioned at A6, and possible bystander site is at A14. PAM is indicated. The R634Q nucleotide sequence shown in SEQ ID NO: 10. The R634Q amino acid sequence shown is SEQ ID NO: 11. The Corrected nucleotide sequence shown in SEQ ID NO: 12. The Corrected amino acid sequence shown is SEQ ID NO: 13. (FIG. 1C) Editing efficiency of adenine (A) to guanine (G) in homozygous R634Q/R634Q iPSCs after ABE correction. Data are expressed as mean±SEM (n=3). (FIG. 1D) Percentage of normal allele in heterozygous (R634Q/+) iPSCs before and after ABE correction. Data are expressed as mean±SEM (n=3). Unpaired Student's t test was performed. p-value ****P<0.0001. (FIG. 1E) Immunocytochemistry of normal (WT), R634Q/+, R634Q/R634Q and ABE-corrected R634Q/R634Q iPSC-derived cardiomyocytes. α-Actinin, RBM20 and DAPI. Scale bar, 10 μm.

FIGS. 2A-2F. Correction of the pathological phenotype of iPSC-derived RBM20^{R634Q} cardiomyocytes by adenine base editing. (FIG. 2A) Heatmap of the alternative splicing patterns in normal (WT), heterozygous (R634Q/+), homozygous (R634Q/R634Q) and corrected R634Q/R634Q iPSC-derived cardiomyocytes. (FIG. 2B) Illustration showing alternative splice isoforms of the TTN gene, TTN-N2BA and TTN-N2B. (FIG. 2C) Relative expression of TTN-N2B isoform quantified by qRT-PCR. Data are expressed as mean±SEM (n=3). One-way ANOVA was performed. p-value ****P<0.0001. (FIG. 2D) Percentage of adenine (A) to guanine (G) editing in corrected R634Q/R634Q iPSC-derived cardiomyocytes after AAV6-mediated ABE correction. A6 is on-target site. A14 is bystander site. (FIG. 2E) Quantification of RBM20 subcellular localization in R634Q/R634Q iPSC-derived cardiomyocytes before and after AAV6-mediated ABE correction (n=100-150 total cells in each group; quantification was performed across three independent differentiation). In each pair of columns, the left column is “R634Q/R634Q” and the right column is “Corrected.” Data are expressed as mean±SEM. Two-way ANOVA with Bonferroni's multiple comparison test was performed. p-value ****P<0.0001. (FIG. 2F) Immunocytochemistry of normal (WT), R634Q/R634Q and AAV6-ABE-corrected R634Q/R634Q iPSC-derived cardiomyocytes. α-Actinin, RBM20 and DAPI. Scale bar, 10 μm.

FIGS. 3A-3C. Cardiac dysfunction in Rbm20^{R636Q} mice. (FIG. 3A) Representative M-mode echocardiographic tracings from 4-week-old wild type (WT), heterozygous (R636Q/+) and homozygous (R636Q/R636Q) mice. (FIG. 3B) Fractional shortening (FS), left ventricular end systolic (LVIDs) and diastolic (LVIDd) diameters measured by echocardiography. Data are expressed as mean±SEM (n=6 per genotype). One-way ANOVA was performed. p-value *P<0.05 and ****P<0.0001. (FIG. 3C) Representative hearts from 12-week-old mice of indicated genotypes. Scale bar, 1 mm.

FIGS. 4A-4E. Systemic delivery of adenine base editing components with AAV9 restored cardiac function. (FIG. 4A) Percentage of adenine (A) to guanine (G) editing in cDNA from hearts of homozygous (R636Q/R636Q) mutant mice at 6-weeks post-ABE correction. A6 is on-target site. A14 is bystander site. A4 and A13 are silent mutations (brown). Data are expressed as mean±SEM (n=4). (FIG. 4B) Fractional shortening (FS), left ventricular end systolic (LVIDs) and diastolic (LVIDd) diameters measured by echocardiography at 4- and 8-weeks post-ABE correction in wild type (WT), homozygous (R636Q/R636Q) and ABE-corrected R636Q/R636Q (Corrected) mice. Data are expressed as mean±SEM (n=6 per group). Two-way ANOVA with turkey's multiple comparison test was performed. In each triplet of columns, the left column is “WT”, the middle column is “R636Q/R636Q”, and the right column is “Corrected”. p-value *P<0.05, **P<0.01 and ****P<0.0001. (FIG. 4C) H&E staining of four-chamber histological sections from WT, R636Q/R636Q and ABE-corrected R636/R636Q mice at 12-weeks post-ABE correction. Scale bar, 1 mm. (FIG. 4D) Kaplan-Meier survival curve of WT (n=15), R636Q/R636Q (n=16) and ABE-corrected R636Q/R636Q mice (n=15). Log-rank (Mantel-Cox) test was performed. p-value ****p<0.0001 for WT or ABE-corrected R636Q/R636Q versus R636Q/R636Q comparison. (FIG. 4E) Immunohistochemistry showing the translocation of RBM20 in cardiomyocytes of WT, R636Q/R636Q and ABE-corrected R636Q/R636Q mice at 12-weeks post-ABE correction. Cardiac troponin T, RBM20 and DAPI. Scale bar, 10 μm.

FIGS. 5A-5B. Generating RBM20^{R634Q} isogenic iPSC lines. (FIG. 5A) Sequence of sgRNA targeting exon 9 of the human RBM20 gene. PAM (CGG) is highlighted. The sequence of the nucleic acid is shown in SEQ ID NO: 14. (FIG. 5B) Sanger sequence of the genomic region spanning the RBM20^{R634Q} mutation (underlined) in normal (WT; SEQ ID NO: 15), heterozygous (R634Q/+; SEQ ID NO: 16) and homozygous (R634Q/R634Q; SEQ ID NO: 17) iPSC lines.

FIGS. 6A-C. Adenine base editing using ABE8e-SpCas9 variants and ABE8e-SaCas9. (FIG. 6A) Percentage of adenine (A) to guanine (G) editing in R634Q/R634Q iPSCs after ABE correction using sgRNA1 and ABE8e-NG-SpCas9 or ABE8e-VRQR-SpCas9. A6 is on-target site. A14 is bystander site. Data are expressed as mean±SEM (n=3). (FIG. 6B) Illustration showing the binding positions of sgRNA2, 3 and 4 in the region of the RBM20^{R634Q} mutation. On-target site and bystander site are indicated. The nucleic acid sequence shown is SEQ ID NO: 10. The amino acid sequence shown is SEQ ID NO: 11. (FIG. 6C) Percentage of adenine (A) to guanine (G) editing in R634Q/R634Q iPSCs after ABE correction using sgRNA2,3 and 4 coupled with each ABE8e base editor. On-target site. Bystander site. Data are expressed as mean±SEM (n=3).

FIG. 7. Recovery of TTN alternative splicing in iPSC-derived cardiomyocytes. Splicing pattern of the TTN gene as measured by percent spliced in (PSI) indicates exon-inclusion ratio. ABE corrected R634Q/R634Q iPSC-derived cardiomyocytes show recovery of TTN splicing.

FIGS. 8A-8C. Adenine base editing restored gene expression in iPSC-derived cardiomyocytes. (FIG. 8A) Heatmap showing differentially regulated gene expression of normal (WT), R634Q/+, R634Q/R634Q and ABE-corrected R634Q/R634Q iPSC-derived cardiomyocytes. (FIGS. 8B and 8C) Gene Ontology terms associated with the up- and down-regulated genes in R634Q/R634Q iPSC-derived cardiomyocytes compared to normal iPSC-derived cardiomyocytes. RNA-sequencing was performed on 3 independent differentiated iPSC-derived cardiomyocytes at day 40 post-differentiation.

FIGS. 9A-9B. Adenine base editing restored abnormalities of calcium handling in iPSC-derived cardiomyocytes. Quantification of (FIG. 9A) the calcium release phase by time to peak and (FIG. 9B) calcium reuptake phase by tau (n=50 cells in each group; quantification was performed across three independent differentiation). Data are shown as means±SEM. One-way ANOVA was performed. p-value ****P<0.0001.

FIGS. 10A-10B. Off-target analysis of adenine base editing in iPSCs. (FIG. 10A) Top eight predicted off-target sites of sgRNA1 coupled with ABEmax-VRQR-SpCas9. The sequences in the table are, from top to bottom, SEQ ID NOs: 18-26, respectively. (FIG. 10B) Percentage of adenine (A) to guanine (G) editing determined by Sanger sequencing in the top eight predicted off-target sites. Data are expressed as mean±SEM (n=3). One-way ANOVA was performed. p-value ****P<0.0001.

FIGS. 11A-11D. AAV6-mediated adenine base editing restored the nuclear localization of RBM20 in differentiated iPSC-derived cardiomyocytes. (FIG. 11A) Illustration of dual AAV vectors used to deliver ABE components to iPSC-derived cardiomyocytes. ABEmax, VRQR-SpCas9 and inteins (Int) are driven by the cardiac troponin T promoter. sgRNA expression cassette is driven by U6 RNA polymerase III promoter. AAV serotype 6 was used for differentiated iPSC-derived cardiomyocytes. (FIG. 11B) Schematic showing experimental design of ABE delivery by AAV6 into differentiated iPSC-derived cardiomyocytes. (FIG. 11C) Representative Sanger sequence of the genomic region of the RBM20^{R634Q} mutation (underlined) in normal (WT; SEQ ID NO: 27), uncorrected (R634Q/R634Q; SEQ ID NO: 28), and ABE-corrected homozygous (Corrected; SEQ ID NO: 27) iPSC-derived cardiomyocytes. (FIG. 11D) Immunocytochemistry of normal (WT), R634Q/R634Q and ABE-corrected R634Q/R634Q iPSC-derived cardiomyocytes. α-Actinin, RBM20 and DAPI. Scale bar, 20 μm.

FIGS. 12A-12C. Generating a knock-in mouse model carrying the Rbm20^{R636Q} mutation. (FIG. 12A) Sequence of sgRNA targeting exon 9 of the Rbm20 gene. PAM is highlighted. The nucleic acid shown is SEQ ID NO: 29. (FIG. 12B) Illustration showing the nucleotide and amino acid sequences around the genomic region of the Rbm20^{R636Q} mutation. Knock-in Rbm20^{R636Q} mutation replaces nucleotides shown. The nucleotide sequence shown is SEQ ID NO: 30. The amino acid sequence shown is SEQ ID NO: 31. (FIG. 12C) Sanger sequence of the genomic region of the Rbm20^{R636Q} mutation in wild type (WT; SEQ ID NO: 32), heterozygous (R636Q/+; SEQ ID NO: 33) and homozygous (R636Q/R636Q; SEQ ID NO: 34) mice.

FIGS. 13A-13B. Strategy for adenine base editing in homozygous R636Q/R636Q mice. (FIG. 13A) Illustration depicting adenine base editing (ABE) correction of the R636Q mutation (nucleic acid sequenced is SEQ ID NO: 30; amino acid sequence is SEQ ID NO: 31) using sgRNA and ABEmax-VRQR-SpCas9. On-target site is positioned at A6. Bystander mutation is at A14 and A20. Silent mutations are located at A4, A13 and A19. PAM is indicated. Corrected nucleic acid sequence is SEQ ID NO: 35. Corrected amino acid sequence is SEQ ID NO: 36. (FIG. 13B) Strategy for systemic delivery of AAV9-mediated ABE correction. R636Q/R636Q mice were injected intraperitoneally with 2.5×10¹⁴ vg/kg of total AAV9 components at postnatal day 5.

FIGS. 14A-14C. Correction of the Rbm20^{R636Q} mutation by AAV9-mediated adenine base editing in vivo. Percentage of adenine (A) to guanine (G) editing in (FIG. 14A) DNA and (FIG. 14B) cDNA from the whole hearts of ABE-corrected R636Q/R636Q (Corrected) mice at 6-weeks post AAV9-mediated ABE correction. Data are expressed as mean±SEM (n=4). Unpaired Student's t test was performed. p-value ****P<0.0001. (FIG. 14C) Sanger sequencing showing the region of the Rbm20^{R636Q} mutation of cDNA in WT (SEQ ID NO: 37), R636Q/R636Q (SEQ ID NO: 38) and ABE-corrected R636Q/R636Q (SEQ ID NO: 39) mice.

FIG. 15. Systemic delivery of ABE components rescued cardiac function in homozygous R636Q/R636Q mice. M-mode echocardiographic tracings of wild type (WT), homozygous (R636Q/R636Q) and ABE-corrected R636Q/R636Q (Corrected) mice at 8-weeks post AAV9-mediated ABE correction.

FIGS. 16A-16B. Histological analysis of homozygous R636Q/R636Q mouse hearts. (FIG. 16A) H&E staining and (FIG. 16B) Masson's trichrome staining of left ventricle in normal (WT), R636Q/R636Q and ABE-corrected R636Q/R636Q (Corrected) mice at 12-weeks post-AAV9 administration. Scale bar, 50 μm.

FIGS. 17A-17B. Adenine base editing partially restored the alternative splicing of the titin (Ttn) gene. Relative expression of (FIG. 17A) the N2B isoform and (FIG. 17B) the N2BA isoform of titin (Ttn) gene was quantified by qRT-PCR in WT, R636Q/R636Q and ABE-corrected R636Q/R636Q mice at 6-weeks post AAV9-mediated ABE correction. Data are expressed as mean±SEM (n=4). One-way ANOVA was performed. p-value ***P<0.001 and ****P<0.0001.

FIGS. 18A-18C. Adenine base editing restored transcriptional expression in the heart. (FIG. 18A) Heatmap showing the expression of differentially regulated genes in WT, R636Q/R636Q and ABE-corrected R636/R636Q mice at 6-weeks post AAV9-mediated ABE correction (n=4). (FIGS. 18B and 18C) Gene Ontology terms associated with the up- and down-regulated genes in R636Q/R636Q mice compared to WT mice.

FIGS. 19A-19D. Prime editing of the RBM20^{R636S} mutation in iPSCs. (FIG. 19A) Illustration of the prime editing (PE) strategy for correction of the RBM20^{R636S} mutation (sequence shown is SEQ ID NO: 40). Prime editing guide RNA (pegRNA) contains a spacer (SEQ ID NO: 5), prime binding site (PBS, 11nt length; SEQ ID NO: 6) and reverse transcriptase template (RT, 17nt length; SEQ ID NO: 7). The RBM20^{R636S} mutation and intended edited nucleotides are colored red. Silent mutation for disrupting the PAM is colored blue. The nicking site of pegRNA is indicated by a green arrowhead. The second nicking site of the sgRNA is indicated by an arrowhead. (FIG. 19B) Sanger sequence of the genomic region of the RBM20^{R636S} mutation (underlined) in normal (WT; SEQ ID NO: 41), uncorrected (R636S/R636S; SEQ ID NO: 42) and PE-corrected (SEQ ID NO: 43) iPSC lines. (FIG. 19C) Percentage of adenine (A) to cytosine (C) editing after PE3b correction and PE3bmax coupled with engineered pegRNA (epegRNA) correction in R636S/R636S iPSCs. Data are expressed as mean±SEM (n=3). Unpaired Student's t test was performed. p-value ***P<0.001. (FIG. 19D) Immunocytochemistry of normal (WT), R636S/R636S and PE-corrected R636S/R636S iPSC-derived cardiomyocytes. α-Actinin, RBM20 and DAPI. Scale bar, 10 μm.

FIG. 20 depicts a representative schematic diagram illustrating the making of a human cell line model containing the RBM20 R634Q mutation in patient-derived induced pluripotent stem cells (iPSCs) according to various aspects of the disclosure. The human nucleotide sequence shown is SEQ ID NO: 44. The amino acid sequence shown is SEQ ID NO: 45. The WT sequence is SEQ ID NO: 15. The R634Q sequence is SEQ ID NO: 17.

FIGS. 21A and 21B depict representative images illustrating immunofluorescent staining of patient-derived induced pluripotent stem cells (iPSCs) containing the RBM20 R634Q mutation differentiated into cardiomyocytes (iPSC-CMs) (FIG. 21B) compared to wild-type cells (FIG. 21A) according to various aspects of the disclosure.

FIG. 22 depicts a representative schematic diagram illustrating an exemplary CRISPR/CAS9 system used for correction of a R634Q mutation of the RBM20 gene in human cell according to various aspects of the disclosure. The human nucleotide sequence shown is SEQ ID NO: 44. The amino acid sequence shown is SEQ ID NO: 45. The WT sequence is SEQ ID NO: 46. The R634Q sequence is SEQ ID NO: 47.

FIG. 23 depicts a representative schematic diagram illustrating a genetically modified mouse line generated to model the human R634Q mutation of the RBM20 gene by targeting the corresponding region (R636Q) in a mouse sequence according to various aspects of the disclosure. The human nucleotide sequence shown is SEQ ID NO: 48. The amino acid sequence shown is SEQ ID NO: 49. The sequence in the chromatogram is SEQ ID NO: 50.

### DETAILED DESCRIPTION

Mutations in RNA binding motif protein 20 (RBM20) are a common cause of dilated cardiomyopathy (DCM) in humans. Provided herein is a CRISPR-Cas9 adenine base editing (ABE) system for correcting the pathogenic R634Q mutation of RBM20. This system was used in human induced pluripotent stem cell-derived cardiomyocytes to restore cardiomyocyte functionality.

The present disclosure is based, at least in part, on the discovery of guide RNAs (gRNAs) for use with Clustered Regularly Interspaced Short Palindromic Repeats (CRISPR)-CRISPR associate protein 9 (Cas9) systems that successfully reverse phenotypes associated with familial cardiomyopathies such as DCM by correcting genetic mutations through base-pair editing. Accordingly, provided herein are compositions comprising single guide RNA (sgRNA) designed for a CRISPR/CAS9 system and method of using thereof for preventing, ameliorating or treating one or more cardiomyopathies.

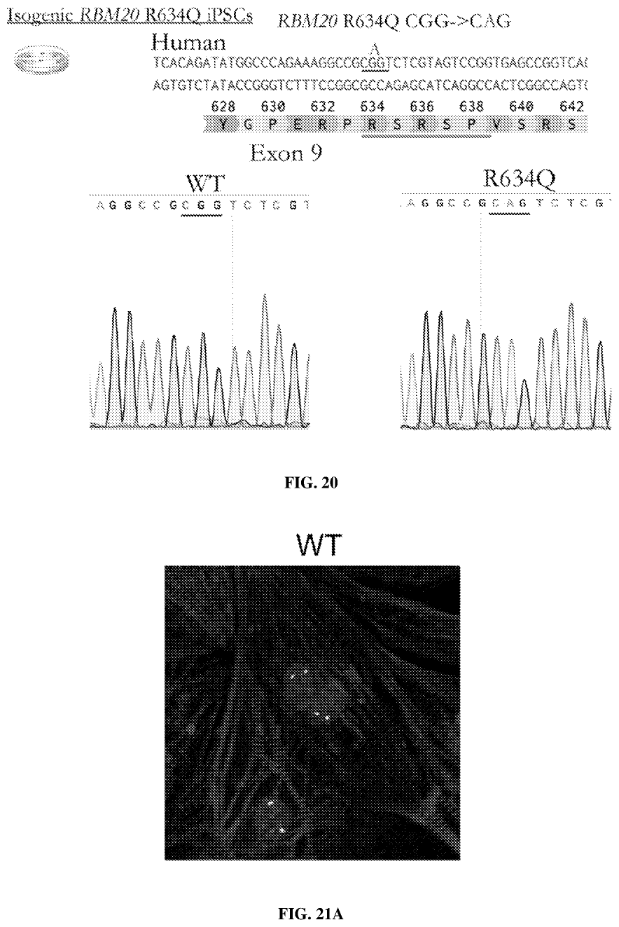

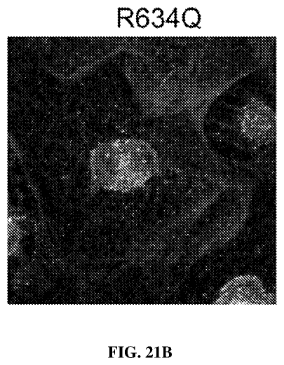

In an exemplary method, CRISPR/CAS9 was used for correction of a RBM20 mutation in human cell. In brief, patient-derived induced pluripotent stem cells (iPSCs) were isolated and used to generate iPSCs containing a RBM20 (R634Q) mutation (Mut) for use in these exemplary studies. RBM20 is a gene that encodes a RNA-binding proteins that regulates titin alternative splicing. Missense mutations in the RS-rich domain of the protein encoded by RBM20 is the underlying cause of familial dilated cardiomyopathy (DCM) in about 6% of patients diagnosed with DCM. As shown in FIG. 20, nucleotides corresponding to “CGG” (encoding for the wild-type amino acid residue “R” at 634) were mutated to “CAG” which encodes for the mutation mimicking the RBM20 mutation of the amino acid residue at 634 of the human sequence to “Q” (R634Q). Next, the patient-derived induced pluripotent stem cells (iPSCs) containing the RBM20 R634Q mutation were differentiated into cardiomyocytes (iPSC-CMs) (FIGS. 21A-21B). Resulting cardiomyocytes of wild-type iPSC-CMs and mutated (R634Q) cells were stained for actin and a protein marker of sarcomeres. FIGS. 21A-21B show sarcomere disruption in R634Q iPSC-CMs.

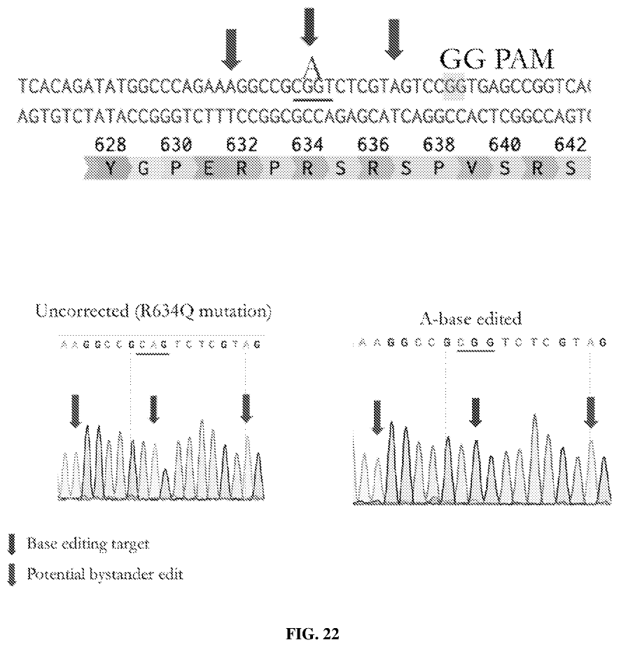

FIG. 22 shows a gRNA with a protospacer adjacent motif (PAM). Following nucleofection of a plasmid encoding the gRNA with the protospacer adjacent motif (PAM) and a plasmid encoding ABEmax-SpCas9-NG a robust editing of the mutant adenine nucleotide back to the wildtype guanine nucleotide was assessed for editing efficiency and showed that the CRISPR/Cas9 system corrected the R634Q mutation with a high efficiency. Next, patient-derived induced pluripotent stem cells (iPSCs) containing the R634Q mutation (Mut) or iPSCs corrected using the CRISPR/CAS9 method described above (Cor) were isolated and differentiated into cardiomyocytes (iPSC-CMs). Analysis of force generation by Mut iPSC-CMs and Cor iPSC-CMs and the effect on cell phenotype were assessed as described in Example 2.

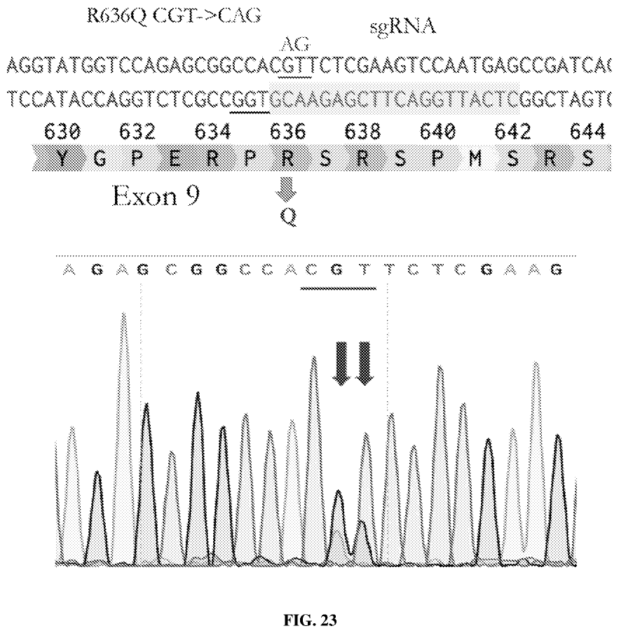

Additionally, a humanized mouse model bearing the corresponding RBM20 mutation was generated, which displayed severe cardiac dysfunction and sudden death, recapitulating the human DCM phenotype. In particular, a genetically modified mouse line was generated to model the human RBM20 R634Q mutation (FIG. 23). Specifically, the mouse line contained the same human disease-causing mutation within the RBM20 gene at R636Q, which corresponds to the human RBM20 R634Q mutation. Mice carrying the missense mutation on one allele and mice carrying the missense mutation on both alleles were monitored for cardiac phenotypes and cardiac fibrosis in comparison to wild type mice. To correct the RBM20 R636Q mutation in the mouse model of the human RBM20 R634Q mutation, a sgRNA was designed for adeno-associated virus (AAV)-based correction in the mouse line. On-target and off-target editing efficiency in the mice was determined using AAV delivery and/or A-base editor. After administering the sgRNA via AAV into the mouse model of the human RBM20 R634Q mutation, cardiac function was assessed and compared to cardiac function prior to administration of sgRNA to measure phenotypic rescue in the mice (FIG. 15). Systemic delivery of ABE components with adeno-associated virus delivery rescued RBM20 cardiomyopathy in vivo.

Furthermore, prime editing (PE) systems are provided to correct other reported RBM20 mutations that are not correctable by ABE. These findings provide a promising therapeutic strategy for permanent correction of RBM20 mutations and other genetic mutations underlying DCM.

Despite current medical advancements, effective treatment for familial cardiomyopathies remains challenging. Precise gene editing technologies, such as BE and PE, provide an innovative opportunity to correct disease-causing mutations in cardiovascular diseases. However, the large size of the BE and PE systems pose challenges in efficient delivery of these gene editing components by AAV. Other delivery methods such as nanoparticles may solve such a bottleneck. Nevertheless, our study provides a proof-of-concept strategy to correct disease-causing genetic mutations of RBM20 precisely and permanently, and represents an advancement toward therapeutic translation of CRISPR-Cas9 gene editing for DCM.

### I. Dilated Cardiomyopathy

Dilated cardiomyopathy (DCM) is characterized by left ventricular dilation with systolic dysfunction, affecting approximately one person in 2500. This disorder is highly genetically heterogeneous with mutations in 40 different genes, including many encoding sarcomeric and other structural proteins. In some cases, the disorder is caused by mutations in the gene RNA binding motif protein 20 (RBM20) (see GenBank Accession No. NM_001134363.3, which is incorporated herein by reference), located on human chromosome 10, which codes for the protein RBM20 (GenBank Accession No. NP_001127835.2), the sequence of which is reproduced below:

(SEQ ID NO: 51)
MVLAAAMSQDADPSGPEQPDRVACSVPGARASPAPSGPRGMQQPPPPPQP
PPPPQAGLPQIIQNAAKLLDKNPFSVSNPNPLLPSPASLQLAQLQAQLTL
HRLKLAQTAVINNTAAATVLNQVLSKVAMSQPLFNQLRHPSVITGPHGHA
GVPQHAAAIPSTRFPSNAIAFSPPSQTRGPGPSMNLPNQPPSAMVMHPFT
GVMPQTPGQPAVILGIGKTGPAPATAGFYEYGKASSGQTYGPETDGQPGF
LPSSASTSGSVTYEGHYSHTGQDGQAAFSKDFYGPNSQGSHVASGFPAEQ
AGGLKSEVGPLLQGTNSQWESPHGFSGQSKPDLTAGPMWPPPHNQPYELY
DPEEPTSDRTPPSFGGRLNNSKQGFIGAGRRAKEDQALLSVRPLQAHELN
DFHGVAPLHLPHICSICDKKVFDLKDWELHVKGKLHAQKCLVFSENAGIR
CILGSAEGTLCASPNSTAVYNPAGNEDYASNLGTSYVPIPARSFTQSSPT
FPLASVGTTFAQRKGAGRVVHICNLPEGSCTENDVINLGLPFGKVTNYIL
MKSTNQAFLEMAYTEAAQAMVQYYQEKSAVINGEKLLIRMSKRYKELQLK
KPGKAVAAIIQDIHSQRERDMFREADRYGPERPRSRSPVSRSLSPRSHTP
SFTSCSSSHSPPGPSRADWGNGRDSWEHSPYARREEERDPAPWRDNGDDK
RDRMDPWAHDRKHHPRQLDKAELDERPEGGRPHREKYPRSGSPNLPHSVS
SYKSREDGYYRKEPKAKSDKYLKQQQDAPGRSRRKDEARLRESRHPHPDD
SGKEDGLGPKVTRAPEGAKAKQNEKNKTKRTDRDQEGADDRKENTMAENE
AGKEEQEGMEESPQSVGRQEKEAEFSDPENTRTKKEQDWESESEAEGESW
YPTNMEELVTVDEVGEEEDFIVEPDIPELEEIVPIDQKDKICPETCLCVT
TTLDLDLAQDFPKEGVKAVGNGAAEISLKSPRELPSASTSCPSDMDVEMP
GLNLDAERKPAESETGLSLEDSDCYEKEAKGVESSDVHPAPTVQQMSSPK
PAEERARQPSPFVDDCKTRGTPEDGACEGSPLEEKASPPIETDLQNQACQ
EVLTPENSRYVEMKSLEVRSPEYTEVELKQPLSLPSWEPEDVFSELSIPL
GVEFVVPRTGFYCKLCGLFYTSEETAKMSHCRSAVHYRNLQKYLSQLAEE
GLKETEGADSPRPEDSGIVPRFERKKL

The murine Rbm20 protein is encoded by GenBank Accession No. NM_001170847.1, which is incorporated herein by reference, and has the following amino acid sequence (GenBank Accession No. NP_001164318.1):

(SEQ ID NO: 52)
MVLAVAMSQDADPSGPEQPDRDACVMPGVQGPSVPQGQQGMQPLPPPPPP
QPQASLPQIIQNAAKLLDKSPFSVNNQNPLLTSPASVQLAQIQAQLTLHR
LKMAQTAVTNNTAAATVLNQVLSKVAMSQPLFNQLRHPSVLGTAHGPTGV
SQHAASVPSAHFPSTAIAFSPPSQTGGPGPSVSLPSQPPNAMVVHTFSGV
VPQTPAQPAVILSLGKAGPTPATTGFYDYGKANSGQAYGSETEGQPGFLP
ASASATASGSMTYEGHYSHTGQDGQPAFSKDFYGPNAQGPHIAGGFPADQ
TGSMKGDVGGLLQGTNSQWERPPGFSGQNKPDITAGPSLWAPPASQPYEL
YDPEEPTSDRAPPAFGSRLNNSKQGFGCSCRRTKEGQAVLSVRPLQGHQL
NDFRGLAPLHLPHICSICDKKVFDLKDWELHVKGKLHAQKCLLFSESAGL
RSIRASGEGTLSASANSTAVYNPTGNEDYTSNLGTSYAAIPTRAFAQSNP
VFPSASSGTSFAAQRKGAGRVVHICNLPEGSCTENDVINLGLPFGKVTNY
ILMKSTNQAFLEMAYTEAAQAMVQYYQEKPAIINGEKLLIRMSTRYKELQ
LKKPGKNVAAIIQDIHSQRERDMLREADRYGPERPRSRSPMSRSLSPRSH
SPPGPSRADWGNGRDSYAWRDEDRETVPRRENGEDKRDRLDVWAHDRKHY
PRQLDKAELDERLEGGRGYREKYLKSGSPGPLHSVSGYKGREDGYHRKEP
KAKLDKYPKQQPDVPGRSRRKEEARLREPRHPHPEDSGKAEDLEPKITRA
PDGTKSKQSEKSKTKRADRDQEGADDKKESQLAENEAGAEEQEGMVGIQQ
EGTESCDPENTRTKKGQDCDSGSEPEGDNWYPTNMEELVTVDEVGEEDFI
MEPDLPELEEIVPIDQKDKTLPKICTCVTATLGLDLAKDFTKQGETLGNG
DAELSLKLPGQVPSTSASCPNDIDLEMPGLNLDAERKPAESETGLSLEVS
NCYEKEARGEEDSDVSLAPAVQQMSSPQPADERARQSSPFLDDCKARGSP
EDGSHEASPLEGKASPPTESDLQSQACRENPRYMEVKSLNVRSPEFTEAE
LKEPLSLPSWEPEVFSELSIPLGVEFVVPRTGFYCKLCGLFYTSEEAAKV
SHCRSTVHYRNLQKYLSQLAEEGLKETEGTDSPSPERGGIGPHLERKKL

Dilated cardiomyopathy (DCM) is one of the most common causes of heart failure, an increasingly pandemic condition characterized by impaired cardiac performance and high morbidity and mortality. Dilated cardiomyopathy is defined by the presence of left ventricular (LV) enlargement and contractile dysfunction together with accumulation of interstitial fibrosis. Dilated cardiomyopathy patients are also at high risk of ventricular arrhythmias and sudden death. As heart failure progresses, treatment options, including evidence-based polypharmacy and cardiac resynchronisation therapy, may become ineffective, leaving heart transplant as a final resort available only to very few. Five year mortality after initial diagnosis of heart failure remains approximately 50%. Genetic variations in more than 50 genes have been implicated as causative in dilated cardiomyopathy. About 25 to 35% of affected individuals have familial forms of the disease, with most mutations affecting genes encoding cytoskeletal proteins, while some affect other proteins involved in contraction.

Dilated cardiomyopathy is a heterogeneous disease. In particular, it is known that an ischemic form of dilated cardiomyopathy exists, as well as a non-ischemic form, a variant of which is non-ischemic cardiomyopathy associated with atherosclerosis. In ischemic dilated cardiomyopathy, a coronary artery disease is regarded as being the underlying cause. In non-ischemic dilated cardiomyopathy, coronary artery disease is not regarded as the principal cause underlying the cardiomyopathy, but genetic, metabolic and inflammatory states instead. Partly, pathological states following hypertrophy in valve diseases or arterial hypertrophy may also cause non-ischemic dilated cardiomyopathy. Non-ischemic cardiomyopathy associated with atherosclerosis is particularly hard to diagnose, as the atherosclerosis is not the cause underlying the cardiomyopathy which is observed.

Dilated cardiomyopathy can easily be diagnosed using echocardiography. Echocardiography, however, does not give information of the cause underlying cardiomyopathy. This holds in particular true in cases when two or more causes are to be taken into consideration, e.g., in diabetes. Moreover, the present methods which are mostly invasive methods cannot describe the mechanism responsible for the progress of the disease.

### II. CRISPR Systems

The present disclosure provides for compositions for preventing, ameliorating or treating one or more cardiomyopathies. In some embodiments, compositions herein can include a guide RNA (gRNA). In some embodiments, compositions herein can include a Clustered Regularly Interspaced Short Palindromic Repeats (CRISPR)/CRISPR associate protein 9 (Cas9) system. In some embodiments, compositions herein can include AAV vectors, AAV viral particles, or a combination thereof for delivery of gRNA and/or CRISPR/Cas 9 systems disclosed herein. In some embodiments, compositions herein can be formulated to form one or more pharmaceutical compositions.

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

CRISPR/CAS9 systems can be naturally occurring defense mechanisms in prokaryotes that have been repurposed as an RNA-guided DNA-targeting platform used for gene editing. CRISPR/CAS9 systems relies on the DNA nuclease Cas9, and two noncoding RNAs, crisprRNA (crRNA) and trans-activating RNA (tracrRNA) (i.e., gRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR/CAS9 complex through Watson-Crick base pairing typically with a 20 nucleotide (nt) sequence in the target DNA. Changing the sequence of the 5′ 20 nt in the crRNA allows targeting of the CRISPR/CAS9 complex to specific loci. The CRISPR/CAS9 complex only binds DNA sequences that contain a sequence match to the first 20 nt of the crRNA, if the target sequence is followed by a specific short DNA motif (with the sequence NGG) referred to as a protospacer adjacent motif (PAM). TracrRNA hybridizes with the 3′ end of crRNA to form an RNA-duplex structure that is bound by the Cas9 endonuclease to form the catalytically active CRISPR/CAS9 complex, which can then cleave the target DNA. Once the CRISPR/CAS9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end). After binding of CRISPR/CAS9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some embodiments, the Cas9 (CRISPR associated protein 9) endonuclease can be used in a CRISPR method herein for preventing, ameliorating or treating one or more cardiomyopathies as described herein. A “Cas9 molecule,” as used herein, refers to a molecule that can interact with a gRNA molecule and, in concert with the gRNA molecule, localize (e.g., target or home) to a site which comprises a target sequence and PAM sequence. Cas9 proteins are known to exist in many CRISPR systems including, but not limited to: Methanococcus maripaludis; Corynebacterium diphtheriae; Corynebacterium efficiens; Corynebacterium glutamicum; Corynebacterium kroppenstedtii; Mycobacterium abscessus; Nocardia farcinica; Rhodococcus erythropolis; Rhodococcus jostii; Rhodococcus opacus; Acidothermus cellulolyticus; Arthrobacter chlorophenolicus; Kribbella flavida; Thermomonospora curvata; Bifidobacterium dentium; Bifidobacterium longum; Slackia heliotrinireducens; Persephonella marina; Bacteroides fragilis; Capnocytophaga ochracea; Flavobacterium psychrophilum; Akkermansia muciniphila; Roseiflexus castenholzii; Roseiflexus; Synechocystis; Elusimicrobium minutum; Fibrobacter succinogenes; Bacillus cereus; Listeria innocua; Lactobacillus casei; Lactobacillus rhamosus; Lactobacillus salivarius; Streptococcus agalactiae; Streptococcus dysgalactiae equisimilis; Streptococcus equi zooepidemicus; Streptococcus gallolyticus; Streptococcus gordonii; Streptococcus mutans; Streptococcus pyogenes; Streptococcus pyogenes M1 GAS; Streptococcus pyogenes MGAS5005; Streptococcus pyogenes MGAS2096; Streptococcus pyogenes MGAS9429; Streptococcus pyogenes MGAS 10270; Streptococcus pyogenes MGAS6180; Streptococcus pyogenes MGAS315; Streptococcus pyogenes SSI-1; Streptococcus pyogenes MGAS 10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EFO1-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis; Francisella tularensis WY96-3418; and Treponema denticola ATCC 35405, and the like.

In some embodiments, a Cas9 enzyme herein may be from Streptococcus, Staphylococcus, or variants thereof. It should be understood, that wild-type Cas9 may be used or modified versions of Cas9 may be used (e.g., evolved versions of Cas9, or Cas9 orthologues or variants), as provided herein. In some aspects, a Cas9 enzyme herein may be a Streptococcus pyogenes Cas9 (SpCas9) variant. In some aspects, a Cas9 enzyme herein may be a Streptococcus pyogenes Cas9 (SpCas9) variant compatible with NGG PAMs. The canonical PAM is the sequence 5′-NGG-3′, where “N” is any nucleobase followed by two guanine (“G”) nucleobases. In some aspects, a Cas9 enzyme herein may be a Streptococcus pyogenes Cas9 (SpCas9) variant compatible with non-NGG PAMs. In some aspects, a Cas9 enzyme herein may be a Streptococcus pyogenes Cas9 (SpCas9) variant compatible with non-NGG PAMs. In some aspects, a Cas9 enzyme herein may be a variant of the adenine base editor (ABE) ABEmax, which uses Streptococcus pyogenes Cas9 (SpCas9) variants compatible with non-NGG PAMs. In some examples, a Cas9 enzyme herein may be ABEmax-SpCas9-NG.

In some embodiments, the ability of an active Cas9 molecule to interact with and cleave a target nucleic acid is PAM sequence dependent. A PAM sequence is a sequence in the target nucleic acid. In some embodiments, a PAM herein may have a polynucleotide sequence having at least 85% (e.g., about 85%, 90%, 95%, 99%, 100%) sequence identity with the nucleotide sequence of TGA, CGG, or TGG. In some embodiments, a PAM herein may have the nucleotide sequence of TGA, CGG, or TGG. In some embodiments, cleavage of the target nucleic acid occurs upstream from the PAM sequence. Active Cas9 molecules from different bacterial species can recognize different sequence motifs (e.g., PAM sequences). In some embodiments, an active Cas9 molecule of S. pyogenes can recognize the sequence motif “NGG” and directs cleavage of a target nucleic acid sequence 1 to 10, e.g., 3 to 5, base pairs upstream from that sequence. In some embodiments, an active Cas9 molecule of S. pyogenes can recognize a non-NGG sequence motif and directs cleavage of a target nucleic acid sequence 1 to 10, e.g., 3 to 5, base pairs upstream from that sequence.

In some embodiments, engineered CRISPR gene editing systems herein (e.g., for gene editing in mammalian cells) can include (1) a guide RNA molecule (gRNA) as disclosed herein comprising a targeting domain (which is capable of hybridizing to the genomic DNA target sequence), and sequence which is capable of binding to a Cas, e.g., Cas9 enzyme, and (2) a Cas, e.g., Cas9, protein. This second domain may comprise a domain referred to as a tracr domain. The targeting domain and the sequence which is capable of binding to a Cas, e.g., Cas9 enzyme, may be disposed on the same (sometimes referred to as a single gRNA, chimeric gRNA or sgRNA) or different molecules (sometimes referred to as a dual gRNA or dgRNA). If disposed on different molecules, each includes a hybridization domain which allows the molecules to associate, e.g., through hybridization.

In certain embodiments, to generate a double stranded break in the target sequence, CRISPR/Cas9 systems herein can bind to a target sequence as determined by the guide nucleic acid (gRNA), and the nuclease recognizes a protospacer adjacent motif (PAM) sequence adjacent to the target sequence in order to cut the target sequence. In some embodiments, CRISPR/cas9 systems herein can include a scaffold sequence compatible with the nucleic acid-guided nuclease. In other embodiments, the guide sequence can be engineered to be complementary to any desired target sequence for efficient editing of the target sequence. In other embodiments, the guide sequence can be engineered to hybridize to any desired target sequence. In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length.

In some embodiments, a target sequence of CRISPR/cas9 systems herein can be any polynucleotide endogenous or exogenous to a prokaryotic or eukaryotic cell, or in an in vitro system for verification or otherwise. In other embodiments, a target sequence can be a polynucleotide residing in the nucleus of the eukaryotic cell. A target sequence can be a sequence coding a gene product (e.g., a protein) or a non-coding sequence (e.g., a regulatory polynucleotide or a junk DNA). It is contemplated herein that the target sequence should be associated with a PAM; that is, a short sequence recognized by CRISPR/cas9 systems herein. In some embodiments, sequence and length requirements for a PAM differ depending on the nucleic acid-guided nuclease selected. In certain embodiments, PAM sequences can be about 2-5 base pair sequences adjacent the target sequence or longer, depending on the PAM desired. Examples of PAM sequences are given in the Examples section below, and the skilled person will be able to identify further PAM sequences for use with a given nucleic acid-guided nuclease as these are not intended to limit this aspect of the present inventive concept. Further, engineering of a PAM Interacting (PI) domain can allow programming of PAM specificity, improve target site recognition fidelity, and increase the versatility of a nucleic acid-guided nuclease genome engineering platform.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a base editing enzyme or a reverse transcriptase.

The engineered CRISPR technologies of base editing and prime editing have expanded the toolbox of gene editing strategies to potentially correct genetic mutations by enabling precise edits at individual nucleotides (Chemello et al., 2020). In base editing, Cas9 nickase (nCas9) or deactivated Cas9 (dCas9) is fused to a deaminase protein, allowing precise single-base pair conversions without DSBs within a defined editing window in relation to the protospacer adjacent motif (PAM) site of a sgRNA (Rees et al., 2018). There are two major classes of DNA base editors: cytosine base editors (CBEs), which convert a C:G base pair into a T:A base pair, and adenine base editors (ABEs), which convert an A:T base pair into a G:C base pair. As such, base editors allow efficient installation of single base substitutions in DNA. For example, adenosine deaminases induce adenosine (A) to inosine (I) edits in single-stranded DNA that in turn result in A-to-G transitions after DNA repair or replication. Adenine base editors (ABEs) are fusions of programmable DNA-binding domains (e.g., catalytically impaired RNA-guided CRISPR/Cas nucleases) linked to an engineered adenosine deaminase. In instances where the programmable DNA-binding domain is a CRISPR/Cas nuclease, targeted adenines lie within an “editing window” in the single-stranded (ss) DNA bubble (R-loop) induced by the CRISPR-Cas RNA-protein complex. The most commonly used ABEs comprise an adenosine deaminase heterodimer consisting of E. coli TadA (wild type) fused to an engineered E. coli TadA variant (e.g. ABEmax) or a single engineered E. coli TadA variant (e.g. ABE8e, ABE8eV106W, or ABE8.20-m) as well as a nickase Cas9 and nuclear localization sequences (NLS). ABEs have been used successfully for installation of A-to-G substitutions in multiple cell types and organisms and could potentially reverse a large number of mutations known to be associated with human disease. Examples of ABEs include those described in U.S. Pat. Publn. US20200308571, PCT Publn. WO2020214842, and PCT Publn. WO2021025750, which are each incorporated herein by reference in their entirety. Reference is made to International Publication No. WO 2018/027078, published Aug. 2, 2018; International Publication No. WO 2019/079347 published Apr. 25, 2019; International Publication No. WO 2019/226593, published Nov. 28, 2019; U.S. Patent Publication No. 2018/0073012, published Mar. 15, 2018, which issued as U.S. Pat. No. 10,113,163, on Oct. 30, 2018; and U.S. Patent Publication No. 2017/0121693, published May 4, 2017, which issued as U.S. Pat. No. 10,167,457 on Jan. 1, 2019.

Prime editing is a versatile and precise genome editing method that directly writes new genetic information into a specified DNA site using a CRISPR system working in association with a polymerase (i.e., in the form of a fusion protein or otherwise provided in trans with the CRISPR system), wherein the prime editing system is programmed with a prime editing (pe) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The prime editing system is composed of a prime editing guide RNA (pegRNA) and a nCas9 fused to an engineered reverse transcriptase. The pegRNA consists of (from 5′ to 3′) a sgRNA that anneals to a target site, a scaffold for the nCas9, a reverse transcription template (RT template) containing the desired edit, and a primer binding site (PBS) that binds to the non-target strand. The RT template can be programmed to introduce any type of edit, including all possible base transitions and transversions, and insertions and deletions of nucleotides of any length. As such, prime editors allow for prime editing on a target nucleotide sequence in the presence of a pegRNA (or “extended guide RNA”). The term “prime editor” refers to fusion constructs comprising a Cas9 nickase and a reverse transcriptase. The prime editing system is further enhanced by including an additional nicking sgRNA that increases editing efficiency by favoring DNA repair to replace the non-edited strand. As such, the term “prime editor” may refer to the fusion protein or to the fusion protein complexed with a pegRNA, and/or further complexed with a second-strand nicking sgRNA. In some embodiments, the prime editor may also refer to the complex comprising a fusion protein (reverse transcriptase fused to a Cas9), a pegRNA, and a regular guide RNA capable of directing the second-site nicking step of the non-edited strand as described herein. In other embodiments, the reverse transcriptase component of the “prime editor” may be provided in trans. Further examples of prime editors and their use are provided in PCT Publn. WO2020191249, which is incorporated by reference herein in its entirety.

In some aspects, a Cas nuclease and sgRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. Target sites may be 21, 20, 19, 18, 17, 16, 15, 14, 13, 12, 11, or 10 nucleotides in length. The target site may be selected based on its location immediately 5′ of a protospacer adjacent motif (PAM) sequence, such as typically NGG, NG, NAG, NNNRRT, or NNGG. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. The gRNA may be under the control of a constitutive promoter.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia or S. aureus or S. auricularis or S. lugdunensis). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, a Cas9 polypeptide can be a deactivated (e.g., mutated, dCAs9) Cas9 polypeptide, wherein the deactivated Cas9 does not comprise HNH and/or RuvC nickase activities. The HNH and RuvC motifs have been characterized in S. thermophilus (see, e.g., Sapranauskas et al. Nucleic Acids Res. 39:9275-9282 (2011)) and one of skill would be able to identify and mutate these motifs in Cas9 polypeptides from other organisms. For example, the mutations D10A and H840A completely inactivate the nuclease activity of S. pyogenes Cas9. Notably, a Cas9 polypeptide in which the HNH motif and/or RuvC motif is/are specifically mutated so that the nickase activity is reduced, deactivated, and/or absent, can retain one or more of the other known Cas9 functions including DNA, RNA and PAM recognition and binding activities and thus remain functional with regard to these activities, while non-functional with regard to one or both nickase activities.

In an alternative embodiment, the CRIPSR enzyme is a Cas protein, preferably Cas9 (having a nucleotide sequence of Genbank accession no NC_002737.2 and a protein sequence of Genbank accession no NP_269215.1). Again, the Cas9 protein may also be modified to improve activity. For example, the Cas9 protein may comprise the D10A amino acid substitution, this nickase cleaves only the DNA strand that is complementary to and recognized by the crRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas 9 protein may also be the VQR or VRQR variant. Alternatively, the Cas9 protein may be xCas9 (a Streptococcus pyogenes variant that can recognize a broad range of PAM sequences including NG, GAA and GAT). In other alternatives, the Cas9 variant is SpCas9-NG (with a relaxed preference to the third nucleotide of the PAM motif, such that the variant can recognize sequences where the PAM motif is NGN rather than NGG), SaCas9 (from S. aureus that can recognize NNGRR(T) PAM sequences; see Ran, F. A. et al. In vivo genome editing using Staphylococcus aureus Cas9. Nature 520, 186-191, doi:10.1038/nature14299 (2015)), SaCas9-KKH (a variant from S. aureus that can recognize NNNRRT PAM sequences), SauCas9 (from S. auricularis that can recognize NNGG PAM sequences; Genbank accession no WP_107392933.1), or SlugCas9 (from S. lugdunensis M23590 that can recognize NNGG PAM sequences; Genbank accession no WP_002460848.1).

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Each of the guide sequences of Table 2 may further comprise additional nucleotides to form or encode a crRNA, e.g., using any known sequence appropriate for the Cas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains.

In general, a guide polynucleotide can complex with a compatible nucleic acid-guided nuclease and can hybridize with a target sequence, thereby directing the nuclease to the target sequence. A subject nucleic acid-guided nuclease capable of complexing with a guide polynucleotide can be referred to as a nucleic acid-guided nuclease that is compatible with the guide polynucleotide. In addition, a guide polynucleotide capable of complexing with a nucleic acid-guided nuclease can be referred to as a guide polynucleotide or a guide nucleic acid that is compatible with the nucleic acid-guided nucleases.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

The guide RNA can be considered to comprise a scaffold sequence necessary for endonuclease binding and a spacer sequence required to bind to the genomic target sequence.

An exemplary scaffold sequence suitable for use with SaCas9 to follow the guide sequence at its 3′ end is: GTTTAAGTACTCTGTGCTGGAAACAGCACAGAATCTACTTAAACAAGGCAAAAT GCCGTGTTTATCTCGTCAACTTGTTGGCGAGA (SEQ ID NO: 54) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 54, or a sequence that differs from SEQ ID NO: 54 by no more than 1, 2, 3, 4, 5, 10, 15, 20, or 25 nucleotides.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity, nucleic acid binding activity, base editing activity, or reverse transcription activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

As an RNA guided protein, Cas9 requires a short RNA to direct the recognition of DNA targets. Though Cas9 preferentially interrogates DNA sequences containing a PAM sequence (e.g., NGG or NG or NNNRRT or NNGG) it can bind here without a protospacer target. However, the Cas9-gRNA complex requires a close match to the gRNA to create a double strand break. CRISPR sequences in bacteria are expressed in multiple RNAs and then processed to create guide strands for RNA. Because eukaryotic systems lack some of the proteins required to process CRISPR RNAs, the synthetic construct gRNA was created to combine the essential pieces of RNA for Cas9 targeting into a single RNA expressed with the RNA polymerase type III promoter U6. Other promoters under the control of RNA Pol III include those for ribosomal 5S rRNA, tRNA and few other small RNAs, RNase P and RNase MRP RNA, 7SL RNA (the RNA component of the signal recognition particles), Vault RNAs, Y RNA, SINEs (short interspersed repetitive elements), 7SK RNA, two microRNAs, several small nucleolar RNAs and several few regulatory antisense RNAs. Synthetic gRNAs are slightly over 100 bp at the minimum length and contain a portion which is targets the 20 or 21 protospacer nucleotides immediately preceding the PAM sequence. The length of the sgRNA can also be shortened at the 5′ with respect to its canonical length to meet specific criteria, e.g. the removal of a stretch of thymines that can inhibit the polymerase type III transcription activity. gRNAs do not contain the PAM sequence.

In some embodiments, a guide polynucleotide (e.g., gRNA) herein can comprise a guide sequence. A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, may be about or more than about 50%, 60%, 75%, 80%, 85%, 90%, 95%, 97.5%, 99%, or more. Optimal alignment can be determined with the use of any suitable algorithm for aligning sequences. In some embodiments, a guide sequence herein can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In other embodiments, a guide sequence herein can be less than about 75, 50, 45, 40, 35, 30, 25, 20 nucleotides in length. Preferably the guide sequence is 10-30 nucleotides long. In some aspects, a guide sequence herein can be 15-20 nucleotides in length.

In some embodiments, a guide polynucleotide (e.g., gRNA) herein can include a scaffold sequence. In general, a “scaffold sequence” can include any sequence that has sufficient sequence to promote formation of a targetable nuclease complex (e.g., a CRISPR/Cas9 system), wherein the targetable nuclease complex includes, but is not limited to, a nucleic acid-guided nuclease and a guide polynucleotide can include a scaffold sequence and a guide sequence. Sufficient sequence within the scaffold sequence to promote formation of a targetable nuclease complex can include a degree of complementarity along the length of two sequence regions within the scaffold sequence, such as one or two sequence regions involved in forming a secondary structure. In some aspects, the one or two sequence regions may be included or encoded on the same polynucleotide. In some aspects, the one or two sequence regions may be included or encoded on separate polynucleotides. Optimal alignment can be determined by any suitable alignment algorithm, and can further account for secondary structures, such as self-complementarity within either the one or two sequence regions. In some embodiments, the degree of complementarity between the one or two sequence regions along the length of the shorter of the two when optimally aligned can be about or more than about 25%, 30%, 40%. 50%, 60%, 70%, 80%, 90%, 95%, 97.5%, 99%, or higher. In some embodiments, at least one of the two sequence regions can be about or more than about 5, 6, 7, 8, 9, 10, 11, 12, 13, 14, 15, 16, 17, 18, 19, 20, 25, 30, 40, 50, or more nucleotides in length.

In some embodiments, a scaffold sequence of a subject guide polynucleotide herein can comprise a secondary structure. In some embodiments, a secondary structure can comprise a pseudoknot region. In some embodiments, binding kinetics of a guide polynucleotide herein to a nucleic acid-guided nuclease is determined in part by secondary structures within the scaffold sequence. In some embodiments, binding kinetics of a guide polynucleotide herein to a nucleic acid-guided nuclease is determined in part by nucleic acid sequence with the scaffold sequence.

In certain embodiments, spacer mutations can be introduced to a plasmid to test when a substitution gRNA sequence is created or a deletion or insertion mutant is created. Each of these plasmid constructs can be used to test genome editing accuracy and efficiency, for example, having a deletion, substitution or insertion. Alternatively, in some embodiments, gRNA constructs created by compositions and methods disclosed herein can be tested for optimal genome editing time on a select target by observing editing efficiencies over pre-determined time periods. In accordance with these embodiments, gRNA constructs created by compositions and methods disclosed herein can be tested for optimal genome editing windows to optimize editing efficiency and accuracy.

Examples of target polynucleotides for use of engineered gRNA disclosed herein can include a sequence/gene or gene segment associated with a signaling biochemical pathway, e.g., a signaling biochemical pathway-associated gene or polynucleotide. Other embodiments contemplated herein concern examples of target polynucleotides for use of engineered gRNA disclosed herein can include those related to a disease-associated gene or polynucleotide.

A “disease-associated” or “disorder-associated” gene or polynucleotide can refer to any gene or polynucleotide which results in a transcription or translation product at an abnormal level compared to a control or results in an abnormal form in cells derived from disease-affected tissues compared with tissues or cells of a non-disease control. It can be a gene that becomes expressed at an abnormally high level; it can be a gene that becomes expressed at an abnormally low level, or where the gene contains one or more mutations and where altered expression or expression of the mutated gene directly correlates with the occurrence and/or progression of a health condition or disorder. A disease or disorder-associated gene can refer to a gene possessing mutation(s) or genetic variation that are directly responsible or is in linkage disequilibrium with a gene(s) that is responsible for the cause or progression of a disease or disorder. The transcribed or translated products can be known or unknown, and can be at a normal or abnormal level.

In some embodiments, a gRNA disclosed herein may target polynucleotides related to a cardiomyopathy-associated gene or polynucleotide. In some aspects, a cardiomyopathy-associated gene or polynucleotide may be a DCM-associated gene or polynucleotide. In some embodiments, a gRNA disclosed herein may target polynucleotides related to a cardiomyopathy-associated gene such as but not limited to RBM20.

In some embodiments, a gRNA disclosed herein may target polynucleotides related to a cardiomyopathy-associated gene or polynucleotide possessing one or more mutation(s). In some embodiments, a gRNA disclosed herein may target polynucleotides related to a cardiomyopathy-associated gene possessing one or more mutation(s) wherein the cardiomyopathy-associated gene can be RBM20. In some other examples, a gRNA disclosed herein may target polynucleotides related to a R634Q mutation in a RBM20 gene or its mammalian equivalent thereof.

In some embodiments, the gRNA targets a site within a wildtype RBM20 gene. In some embodiments, the gRNA targets a site within a mutant RBM20 gene. In some embodiments, the gRNA targets a dystrophin exon. In some embodiments, the gRNA targets a site in a RBM20 exon that is expressed and is present in one or more RBM20 isoform.

In some embodiments, gRNAs of the disclosure comprise a sequence that is complementary to a target sequence within a coding sequence or a non-coding sequence corresponding to the RBM20 gene, and, therefore, hybridize to the target sequence.

In some embodiments, an engineered polynucleotide (gRNA) disclosed herein can be split into fragments encompassing a synthetic tracrRNA and crRNA. In some aspects, a gRNA herein can have at least 85% sequence identity (e.g., about 85%, 90%, 95%, 99%, 100%) with the nucleotide sequence of any one of SEQ ID NOs: 1-5. In some aspects, a gRNA herein can have the nucleotide sequence of any one of SEQ ID NOs: 1-5.

In some embodiments, a nucleic acid may comprise one or more sequences encoding a gRNA. In some embodiments, a nucleic acid may comprise 1, 2, 3, 4, 5, 6, 7, 8, 9, 10, 11, 12, 13, 14, 15, 16, 17, 18, 19, 20, or 21 sequences encoding a gRNA. In some embodiments, all of the sequences encode the same gRNA. In some embodiments, all of the sequences encode different gRNAs. In some embodiments, at least 2 of the sequences encode the same gRNA, for example at least 3, 4, 5, 6, 7, 8, 9, 10, 11, 12, 13, 14, 15, 16, 17, 18, 19, 20 or 21 of the sequences encode the same gRNA.

In some embodiments, a guide polynucleotide (e.g., gRNA) herein can be DNA. In some embodiments, a guide polynucleotide (e.g., gRNA) herein can be RNA. In some embodiments, a guide polynucleotide (e.g., gRNA) herein can be both RNA and DNA. In some embodiments, a guide polynucleotide (e.g., gRNA) herein can include modified or non-naturally occurring nucleotides. In embodiments where a guide polynucleotide herein comprises RNA, the RNA guide polynucleotide can be encoded by a DNA sequence on a polynucleotide molecule such as a plasmid, linear construct, or editing cassette as disclosed herein.

In some embodiments, nucleotide gene editing may be performed in vitro or ex vivo. In some embodiments, cells are contacted in vitro or ex vivo with a nucleotide editing Cas9 and a gRNA that targets a dystrophin site. In some embodiments, the cells are contacted with one or more nucleic acids encoding the Cas9 and the guide RNA. In some embodiments, the one or more nucleic acids are introduced into the cells using, for example, lipofection or electroporation. Nucleotide gene editing may also be performed in zygotes. In embodiments, zygotes may be injected with one or more nucleic acids encoding Cas9 and a gRNA that targets a dystrophin site. The zygotes may subsequently be injected into a host.

In some embodiments, the Cas9 is provided on a vector. In embodiments, the vector contains a Cas9 derived from S. pyogenes (SpCas9). In some embodiments, the vector contains a Cas9 derived from S. aureus (SaCas9). In some embodiments, the vector contains a Cas9 derived from S. auricularis (SauCas9). In some embodiments, the vector contains a Cas9 derived from S. lugdunensis (SlugCas9). In some embodiments, the Cas9 sequence is codon optimized for expression in human cells or mouse cells. In some embodiments, the vector further contains a sequence encoding a fluorescent protein, such as GFP, which allows Cas 9-expressing cells to be sorted using fluorescence activated cell sorting (FACS). In some embodiments, the vector is a viral vector such as an adeno-associated viral vector.

In some embodiments, the gRNA is provided on a vector. In some embodiments, the vector is a viral vector such as an adeno-associated viral vector. In embodiments, the Cas9 and the guide RNA are provided on the same vector. In embodiments, the Cas9 and the guide RNA are provided on different vectors.

Any type of vector, such as any of those described herein, may be used. In some embodiments, the vector is a lipid nanoparticle. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a cardiomyocyte-specific promoter. In some embodiments, the cardiomyocyte-specific promoter is a cardiac troponin T (cTnT) promoter. In any of the foregoing embodiments, the vector may be an adeno-associated virus vector (AAV).

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of U.S. Patent Publication No. 2015/0111955, which is incorporated by reference herein in its entirety), AAV9 vector, AAV9P vector (also known as AAVMYO, see, Weinmann et al., 2020, Nature Communications, 11:5432), Myo-AAV vectors described in Tabebordbar et al., 2021, Cell, 184:1-20 (e.g., MyoAAV 1A, 2A, 3A, 4A, 4C, or 4E), and AAV9-rh74-HB-P1, AAV9-AAA-P1-SG vectors described in WO2022053630. wherein the number following AAV indicates the AAV serotype. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. In some embodiments, the vector is an AAV9 vector.

Efficiency of in vitro or ex vivo nucleotide editing Cas9 may be assessed using techniques known to those of skill in the art, such as the T7 E1 assay or sequencing. Restoration of RBM20 function may be confirmed using techniques known to those of skill in the art, such as RT-PCR, Western blotting, and immunocytochemistry.

In some embodiments, in vitro or ex vivo gene editing is performed in a cardiac cell. In some embodiments, gene editing is performed in iPSC or iCM cells. In embodiments, the iPSC cells are differentiated after gene editing. For example, the iPSC cells may be differentiated into a cardiac cell after editing. In embodiments, the iPSC cells are differentiated into cardiac muscle cells. In embodiments, the iPSC cells are differentiated into cardiomyocytes. iPSC cells may be induced to differentiate according to methods known to those of skill in the art.

In some embodiments, contacting the cell with the nucleotide editing Cas9 and the gRNA restores RBM20 function. In embodiments, cells which have been edited in vitro or ex vivo, or cells derived therefrom, show levels of RBM20 function that are comparable to wildtype cells. In embodiments, the edited cells, or cells derived therefrom, show levels of RBM20 function that are at least 20%, 30%, 40%, 50%, 60%, 70%, 80%, 90%, 95% or any percentage in between of wildtype levels of RMB20 function.

### III. Nucleic Acid Delivery

In some embodiments, expression cassettes are employed to express a protein product, either for subsequent purification and delivery to a cell/subject, or for use directly in a genetic-based delivery approach. Provided herein are expression vectors which contain one or more nucleic acids encoding nucleotide editing Cas9 and at least one RBM20 guide RNA that targets a RBM20 mutation site. In some embodiments, a nucleic acid encoding nucleotide editing Cas9 and a nucleic acid encoding at least one guide RNA are provided on the same vector. In further embodiments, a nucleic acid encoding nucleotide editing Cas9 and a nucleic acid encoding least one guide RNA are provided on separate vectors.

Polynucleotide sequences encoding a component of CRISPR/cas9 systems herein can include one or more vectors. The term “vector” as used herein can refer to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Other vectors (e.g., non-episomal mammalian vectors) can be integrated into the genome of a host cell upon introduction into the host cell. Recombinant expression vectors can include a nucleic acid of the present inventive concept in a form suitable for expression of the nucleic acid in a host cell, can mean that the recombinant expression vectors include one or more regulatory elements, which can be selected on the basis of the host cells to be used for expression, that is operatively-linked to the nucleic acid sequence to be expressed.

Expression requires that appropriate signals be provided in the vectors and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

In some embodiments, a vector can include a regulatory element operably linked to a polynucleotide sequence encoding a Cas9 nuclease herein. The polynucleotide sequence encoding the Cas9 nuclease herein can be codon optimized for expression in particular cells, such as prokaryotic or eukaryotic cells. Eukaryotic cells can be yeast, fungi, algae, plant, animal, or human cells. Eukaryotic cells can be those derived from a particular organism, such as a mammal, including but not limited to human, mouse, rat, rabbit, dog, or non-human mammal including non-human primate. Plant cells can include, without limitation, cells from seeds, suspension cultures, embryos, meristematic regions, callus tissue, leaves, roots, shoots, gametophytes, sporophytes, pollen and microspores.

As used herein, ‘codon optimization’ can refer to a process of modifying a nucleic acid sequence for enhanced expression in the host cells of interest by replacing at least one codon or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.”

In some embodiments, a Cas9 nuclease herein and one or more guide nucleic acids (e.g., gRNA) can be delivered either as DNA or RNA. Delivery of a Cas9 nuclease herein and guide nucleic acid both as RNA (unmodified or containing base or backbone modifications) molecules can be used to reduce the amount of time that the nucleic acid-guided nuclease persist in the cell (e.g. reduced half-life). This can reduce the level of off-target cleavage activity in the target cell. Since delivery of a Cas9 nuclease as mRNA takes time to be translated into protein, an aspect herein can include delivering a guide nucleic acid several hours following the delivery of the Cas9 mRNA, to maximize the level of guide nucleic acid available for interaction with the nucleic acid-guided nuclease protein. In other cases, the Cas9 mRNA and guide nucleic acid can be delivered concomitantly. In other examples, the guide nucleic acid can be delivered sequentially, such as 0.5, 1, 2, 3, 4, or more hours after the Cas9 mRNA.

In some embodiments, guide nucleic acid (e.g., gRNA) in the form of RNA or encoded on a DNA expression cassette can be introduced into a host cell that includes a nucleic acid-guided nuclease encoded on a vector or chromosome. The guide nucleic acid can be provided in the cassette having one or more polynucleotides, which can be contiguous or non-contiguous in the cassette. In some embodiments, the guide nucleic acid can be provided in the cassette as a single contiguous polynucleotide. In other embodiments, a tracking agent can be added to the guide nucleic acid in order to track distribution and activity.

In other embodiments, a variety of delivery systems can be used to introduce a gRNA and/or Cas9 nuclease into a host cell. In accordance with these embodiments, systems of use for embodiments disclosed herein can include, but are not limited to, yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, exosomes.

In some embodiments, methods are provided for delivering one or more polynucleotides, such as or one or more vectors or linear polynucleotides as described herein, one or more transcripts thereof, and/or one or proteins transcribed therefrom, to a host cell. In some aspects, the present inventive concept further provides cells produced by such methods, and organisms can include or produced from such cells. In some embodiments, an engineered nuclease in combination with (and optionally complexed with) a guide nucleic acid is delivered to a cell.

In certain embodiments, conventional viral and non-viral based gene transfer methods can be used to introduce nucleic acids in cells, such as prokaryotic cells, eukaryotic cells, plant cells, mammalian cells, or target tissues. Such methods can be used to administer nucleic acids encoding components of an CRISPR/Cas9 system herein to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Any gene therapy method known in the art is contemplated of use herein. Methods of non-viral delivery of nucleic acids include are contemplated herein. Adeno-associated virus (“AAV”) vectors can also be used to transduce cells with target nucleic acids, e.g., in the in vitro production of nucleic acids and peptides, and for in vivo and ex vivo gene therapy procedures.

In some embodiments, a nucleic acid encoding any of the constructs herein (e.g., gRNA, Cas9) can be delivered to a cell using an adeno-associated virus (AAV). AAVs are small viruses which integrate site-specifically into the host genome and can therefore deliver a transgene. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, any mammalian AAV serotypes can be used herein for delivering the encoding nucleic acids described herein. Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

In some embodiments, polynucleotides disclosed herein (e.g., gRNA, Cas9) can be delivered to a cell using at least one AAV vector. An AAV vector typically comprises a protein-based capsid, and a nucleic acid encapsidated by the capsid. The nucleic acid may be, for example, a vector genome comprising a transgene flanked by inverted terminal repeats. The AAV “capsid” is a near-spherical protein shell that comprises individual “capsid proteins” or “subunits.” AAV capsids typically comprise about 60 capsid protein subunits, associated and arranged with T=1 icosahedral symmetry. When an AAV vector is described herein as comprising an AAV capsid protein, it will be understood that the AAV vector comprises a capsid, wherein the capsid comprises one or more AAV capsid proteins (i.e., subunits). Also described herein are “viral-like particles” or “virus-like particles,” which refers to a capsid that does not comprise any vector genome or nucleic acid comprising a transgene. The virus vectors of the present disclosure can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619. The virus vectors of the present disclosure can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the present inventive concept. Further, the viral capsid or genomic elements can contain other modifications, including insertions, deletions and/or substitutions.

In some embodiments, AAV vectors disclosed herein may be packaged into virus particles which can be used to deliver the genome for transgene expression in target cells. In some embodiments, AAV vectors disclosed herein can be packaged into particles by transient transfection, use of producer cell lines, combining viral features into Ad-AAV hybrids, use of herpesvirus systems, or production in insect cells using baculoviruses.

In some embodiments, methods of generating a packaging cell herein involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

In some embodiments, a host cell is transiently or non-transiently transfected with one or more vectors, linear polynucleotides, polypeptides, nucleic acid-protein complexes, or any combination thereof as described herein. In some embodiments, a cell can be transfected in vitro, in culture, or ex vivo. In some embodiments, a cell can be transfected as it naturally occurs in a subject. In some embodiments, a cell that is transfected can be taken from a subject. In some embodiments, the cell is derived from cells taken from a subject, such as a cell line.

In some embodiments, a cell transfected with one or more vectors, linear polynucleotides, polypeptides, nucleic acid-protein complexes, or any combination thereof as described herein may be used to establish a new cell line can include one or more transfection-derived sequences. In some embodiments, a cell transiently transfected with the components of an engineered nucleic acid-guided nuclease system as described herein (such as by transient transfection of one or more vectors, or transfection with RNA), and modified through the activity of an engineered nuclease complex, may be used to establish a new cell line can include cells containing the modification but lacking any other exogenous sequence.

In some embodiments, one or more vectors described herein may be used to produce a non-human transgenic cell, organism, animal, or plant. In some embodiments, the transgenic animal may be a mammal, such as a mouse, rat, or rabbit. Methods for producing transgenic cells, organisms, plants, and animals are known in the art, and generally begin with a method of cell transformation or transfection, such as described herein.

Some embodiments disclosed herein relate to use of CRISPR/Cas9 systems disclosed herein; for example, in order to target and knock out genes, amplify genes and/or repair particular mutations associated with DNA repeat instability and a medical disorder. In some embodiments, CRISPR/Cas9 systems herein can be used to harness and to correct these defects of genomic instability. In other embodiments, CRISPR/Cas9 systems disclosed herein can be used for correcting defects in the genes associated with a cardiomyopathy.

### A. Regulatory Elements

In some embodiments, a regulatory element can be operably linked to one or more elements of a targetable CRISPR/cas9 system herein so as to drive expression of the one or more components of the targetable CRISPR/cas9 system.

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

In some embodiments, the nucleotide editing Cas9 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2b, HSP70, proliferin, tumor necrosis factor, and/or thyroid stimulating hormone α gene. In some embodiments, the inducer is phorbol ester (TFA), heavy metals, glucocorticoids, poly(rI)x, poly(rc), ElA, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are cardiomyocyte-specific promoters. In some embodiments, the cardiomyocyte-specific promoter is the cardiac troponin T (cTnT) promoter.

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

### B. 2A Peptide

In some embodiments, a 2A-like self-cleaving domain from the insect virus Thosea asigna (TaV 2A peptide) (EGRGSLLTCGDVEENPGP (SEQ ID NO: 55)) is used. These 2A-like domains have been shown to function across eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems has shown greater than 99% cleavage activity. Other acceptable 2A-like peptides include, but are not limited to, equine rhinitis A virus (ERAV) 2A peptide (QCTNYALLKLAGDVESNPGP (SEQ ID NO: 56)), porcine teschovirus-1 (PTV1) 2A peptide (ATNFSLLKQAGDVEENPGP (SEQ ID NO: 57)) and foot and mouth disease virus (FMDV) 2A peptide (PVKQLLNFDLLKLAGDVESNPGP (SEQ ID NO: 58)) or modified versions thereof.

In some embodiments, the 2A peptide is used to express a reporter and a nucleotide editing Cas9 simultaneously. The reporter may be, for example, GFP or mCherry.

Other self-cleaving peptides that may be used include but are not limited to nuclear inclusion protein a (Nia) protease, a P1 protease, a 3C protease, a L protease, a 3C-like protease, or modified versions thereof.

### C. Trans-Splicing Inteins

In some embodiments, trans-splicing inteins are used to permit the covalent splicing of the split nucleotide editing Cas9. Due to delivery size limitation, nucleotide editing Cas9 can be split in N- and C-terminal peptides. Each half of the split nucleotide editing Cas9 when linked to trans-splicing inteins reassemble after translation into a functional nucleotide editing Cas9 that retains similar editing efficiencies compared to its non-split, full-length equivalent.

In some embodiments, the N- and C-terminal peptides of nucleotide editing Cas9 are fused to split DnaE intein halves from N. puntiforme (Npu).

Other trans-splicing inteins that may be used include but are not limited to Sce VMA, Mtu RecA, Ssp DnaE.

### D. Delivery of Expression Vectors

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One method for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation. In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

The adenoviruses of the disclosure are replication defective, or at least conditionally replication defective. The adenovirus may be of any of the 42 different known serotypes or subgroups A-F. Adenovirus type 5 of subgroup C is the preferred starting material in order to obtain the conditional replication-defective adenovirus vector for use in the present disclosure.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes. Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination.

Other viral vectors may be employed as expression constructs in the present disclosure. Vectors derived from viruses such as vaccinia virus, adeno-associated virus (AAV) and herpesviruses may be employed. They offer several attractive features for various mammalian cells.

In embodiments, particular embodiments, the vector is an AAV vector. AAV is a small virus that infects humans and some other primate species. AAV is not currently known to cause disease. The virus causes a very mild immune response, lending further support to its apparent lack of pathogenicity. In many cases, AAV vectors integrate into the host cell genome, which can be important for certain applications, but can also have unwanted consequences. Gene therapy vectors using AAV can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. Recent human clinical trials using AAV for gene therapy in the retina have shown promise. AAV belongs to the genus Dependoparvovirus, which in turn belongs to the family Parvoviridae. The virus is a small (20 nm) replication-defective, nonenveloped virus.

Wild-type AAV has attracted considerable interest from gene therapy researchers due to a number of features. Chief amongst these is the virus's apparent lack of pathogenicity. It can also infect non-dividing cells and has the ability to stably integrate into the host cell genome at a specific site (designated AAVS1) in the human chromosome 19. This feature makes it somewhat more predictable than retroviruses, which present the threat of a random insertion and of mutagenesis, which is sometimes followed by development of a cancer. The AAV genome integrates most frequently into the site mentioned, while random incorporations into the genome take place with a negligible frequency. Development of AAVs as gene therapy vectors, however, has eliminated this integrative capacity by removal of the rep and cap from the DNA of the vector. The desired gene together with a promoter to drive transcription of the gene is inserted between the inverted terminal repeats (ITR) that aid in concatemer formation in the nucleus after the single-stranded vector DNA is converted by host cell DNA polymerase complexes into double-stranded DNA. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. This feature, along with the ability to infect quiescent cells present their dominance over adenoviruses as vectors for human gene therapy.

Use of the AAV does present some disadvantages. The cloning capacity of the vector is relatively limited and most therapeutic genes require the complete replacement of the virus's 4.8 kilobase genome. Large genes are, therefore, not suitable for use in a standard AAV vector. Options are currently being explored to overcome the limited coding capacity. The AAV ITRs of two genomes can anneal to form head to tail concatemers, almost doubling the capacity of the vector. Insertion of splice sites allows for the removal of the ITRs from the transcript.

Because of AAV's specialized gene therapy advantages, researchers have created an altered version of AAV termed self-complementary adeno-associated virus (scAAV). Whereas AAV packages a single strand of DNA and must wait for its second strand to be synthesized, scAAV packages two shorter strands that are complementary to each other. By avoiding second-strand synthesis, scAAV can express more quickly, although as a caveat, scAAV can only encode half of the already limited capacity of AAV. Recent reports suggest that scAAV vectors are more immunogenic than single stranded adenovirus vectors, inducing a stronger activation of cytotoxic T lymphocytes.

The humoral immunity instigated by infection with the wild type is thought to be a very common event. The associated neutralising activity limits the usefulness of the most commonly used serotype AAV2 in certain applications. Accordingly, the majority of clinical trials currently under way involve delivery of AAV2 into the brain, a relatively immunologically privileged organ. In the brain, AAV2 is strongly neuron-specific.

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

The Inverted Terminal Repeat (ITR) sequences comprise 145 bases each. They were named so because of their symmetry, which was shown to be required for efficient multiplication of the AAV genome. The feature of these sequences that gives them this property is their ability to form a hairpin, which contributes to so-called self-priming that allows primase-independent synthesis of the second DNA strand. The ITRs were also shown to be required for both integration of the AAV DNA into the host cell genome (19th chromosome in humans) and rescue from it, as well as for efficient encapsidation of the AAV DNA combined with generation of a fully assembled, deoxyribonuclease-resistant AAV particles.

With regard to gene therapy, ITRs seem to be the only sequences required in cis next to the therapeutic gene: structural (cap) and packaging (rep) proteins can be delivered in trans. With this assumption many methods were established for efficient production of recombinant AAV (rAAV) vectors containing a reporter or therapeutic gene. However, it was also published that the ITRs are not the only elements required in cis for the effective replication and encapsidation. A few research groups have identified a sequence designated cis-acting Rep-dependent element (CARE) inside the coding sequence of the rep gene. CARE was shown to augment the replication and encapsidation when present in cis.

On the “left side” of the genome there are two promoters called p5 and p19, from which two overlapping messenger ribonucleic acids (mRNAs) of different length can be produced. Each of these contains an intron which can be either spliced out or not. Given these possibilities, four various mRNAs, and consequently four various Rep proteins with overlapping sequence can be synthesized. Their names depict their sizes in kilodaltons (kDa): Rep78, Rep68, Rep52 and Rep40. Rep78 and 68 can specifically bind the hairpin formed by the ITR in the self-priming act and cleave at a specific region, designated terminal resolution site, within the hairpin. They were also shown to be necessary for the AAVS1-specific integration of the AAV genome. All four Rep proteins were shown to bind ATP and to possess helicase activity. It was also shown that they upregulate the transcription from the p40 promoter (mentioned below) but downregulate both p5 and p19 promoters.

The right side of a positive-sensed AAV genome encodes overlapping sequences of three capsid proteins, VP1, VP2 and VP3, which start from one promoter, designated p40. The molecular weights of these proteins are 87, 72 and 62 kiloDaltons, respectively. The AAV capsid is composed of a mixture of VP1, VP2, and VP3 totaling 60 monomers arranged in icosahedral symmetry in a ratio of 1:1:10, with an estimated size of 3.9 MegaDaltons.

The cap gene produces an additional, non-structural protein called the Assembly-Activating Protein (AAP). This protein is produced from ORF2 and is essential for the capsid-assembly process. The exact function of this protein in the assembly process and its structure have not been solved to date.

All three VPs are translated from one mRNA. After this mRNA is synthesized, it can be spliced in two different manners: either a longer or shorter intron can be excised resulting in the formation of two pools of mRNAs: a 2.3 kb- and a 2.6 kb-long mRNA pool. Usually, especially in the presence of adenovirus, the longer intron is preferred, so the 2.3-kb-long mRNA represents the so-called “major splice”. In this form the first AUG codon, from which the synthesis of VP1 protein starts, is cut out, resulting in a reduced overall level of VP1 protein synthesis. The first AUG codon that remains in the major splice is the initiation codon for VP3 protein. However, upstream of that codon in the same open reading frame lies an ACG sequence (encoding threonine) which is surrounded by an optimal Kozak context. This contributes to a low level of synthesis of VP2 protein, which is actually VP3 protein with additional N terminal residues, as is VP1.

Since the bigger intron is preferred to be spliced out, and since in the major splice the ACG codon is a much weaker translation initiation signal, the ratio at which the AAV structural proteins are synthesized in vivo is about 1:1:20, which is the same as in the mature virus particle. The unique fragment at the N terminus of VP1 protein was shown to possess the phospholipase A2 (PLA2) activity, which is probably required for the releasing of AAV particles from late endosomes. Muralidhar et al. reported that VP2 and VP3 are crucial for correct virion assembly. More recently, however, Warrington et al. showed VP2 to be unnecessary for the complete virus particle formation and an efficient infectivity, and also presented that VP2 can tolerate large insertions in its N terminus, while VP1 cannot, probably because of the PLA2 domain presence.

The AAV vector may be replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

In some embodiments, a single viral vector is used to deliver a nucleic acid encoding a nucleotide editing Cas9 and at least one gRNA to a cell. In some embodiments, nucleotide editing Cas9 is provided to a cell using a first viral vector and at least one gRNA is provided to the cell using a second viral vector. In some embodiment, the nucleotide editing Cas9 may use a split-intein dual AAV system which reconstitutes the full-length nucleotide editor by protein trans-splicing. In these systems, the Cas9 protein or the base editor is split into two sections, each fused with one part of an intein system (e.g., intein-N and intein-C encoded by dnaEn and dnaEc, respectively). Upon co-expression, the two sections of the Cas9 protein or nucleobase editor are ligated together via intein-mediated protein splicing. See, U.S. Pat. Publn. US20180127780, which is incorporated by reference herein in its entirety.

In some embodiments, a single viral vector is used to deliver a nucleic acid encoding nucleotide editing Cas9 and at least one gRNA to a cell. In some embodiments, nucleotide editing Cas9 is provided to a cell using a first viral vector and at least one gRNA is provided to the cell using a second viral vector. In some embodiment, the nucleotide editing Cas9 may use a split-intein dual AAV system which reconstitutes the full-length nucleotide editor by protein trans-splicing. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, triceps, extensor digitorum longus, diaphragm, or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement), or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ) to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### E. AAV-Cas9 Vectors

In some embodiments, a Cas9 base editor or prime editor may be packaged into an AAV vector. In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11 or any combination thereof.

Exemplary AAV-Cas9 vectors contain two ITR (inverted terminal repeat) sequences which flank a central sequence region comprising the Cas9 sequence. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-Cas9 vector may contain one or more nuclear localization signals (NLS). In some embodiments, the AAV-Cas9 vector contains 1, 2, 3, 4, or 5 nuclear localization signals. Exemplary NLS include the c-myc NLS, the SV40 NLS, the hnRNPAI M9 NLS, the nucleoplasmin NLS, the sequence RMRKFKNKGKDTAELRRRRVEVSVELRKAKKDEQILKRRNV (SEQ ID NO: 59) of the IBB domain from importin-alpha, the sequences VSRKRPRP (SEQ ID NO: 60) and PPKKARED (SEQ ID NO: 61) of the myoma T protein, the sequence PQPKKKPL (SEQ ID NO: 62) of human p53, the sequence SALIKKKKKMAP (SEQ ID NO: 63) of mouse c-abl IV, the sequences DRLRR (SEQ ID NO: 64) and PKQKKRK (SEQ ID NO: 65) of the influenza virus NS1, the sequence RKLKKKIKKL (SEQ ID NO: 66) of the Hepatitis virus delta antigen and the sequence REKKKFLKRR (SEQ ID NO: 67) of the mouse M×1 protein. Further acceptable nuclear localization signals include bipartite nuclear localization sequences such as the sequence KRKGDEVDGVDEVAKKKSKK (SEQ ID NO: 68) of the human poly(ADP-ribose) polymerase or the sequence RKCLQAGMNLEARKTKK (SEQ ID NO: 69) of the steroid hormone receptors (human) glucocorticoid.

In some embodiments, the AAV-Cas9 vector may comprise additional elements to facilitate packaging of the vector and expression of the Cas9. In some embodiments, the AAV-Cas9 vector may comprise a polyA sequence. In some embodiments, the polyA sequence may be a mini-polyA sequence. In some embodiments, the AAV-CAs9 vector may comprise a transposable element. In some embodiments, the AAV-Cas9 vector may comprise a regulator element. In some embodiments, the regulator element is an activator or a repressor.

In some embodiments, the AAV-Cas9 may contain one or more promoters. In some embodiments, the one or more promoters drive expression of the Cas9. In some embodiments, the one or more promoters are cardiomyocyte-specific promoters. Exemplary cardiac-specific promoters include the cardiac troponin T promoter and α-myosin heavy chain promoter.

In some embodiments, the AAV-Cas9 vector may be optimized for production in yeast, bacteria, insect cells, or mammalian cells. In some embodiments, the AAV-Cas9 vector may be optimized for expression in human cells. In some embodiments, the AAV-Cas9 vector may be optimized for expression in a baculovirus expression system.

In some embodiments of the gene editing constructs of the disclosure, the construct comprises or consists of a promoter and a nuclease. In some embodiments, the construct comprises or consists of an cTnT promoter and a Cas9 nuclease. In some embodiments, the construct comprises or consists of an cTnT promoter and a Cas9 nuclease isolated or derived from Staphylococcus pyogenes (“SpCas9”). In some embodiments, the SpCas9 nuclease comprises or consists of a nucleotide sequence at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to

(SEQ ID NO: 70)
GACAAGAAGTACAGCATCGGCCTGGACATCGGCACCAACTCTGTGGGCTGGGCCGTGATCAC
CGACGAGTACAAGGTGCCCAGCAAGAAATTCAAGGTGCTGGGCAACACCGACCGGCACAGCA
TCAAGAAGAACCTGATCGGAGCCCTGCTGTTCGACAGCGGCGAAACAGCCGAGGCCACCCGG
CTGAAGAGAACCGCCAGAAGAAGATACACCAGACGGAAGAACCGGATCTGCTATCTGCAAGA
GATCTTCAGCAACGAGATGGCCAAGGTGGACGACAGCTTCTTCCACAGACTGGAAGAGTCCT
TCCTGGTGGAAGAGGATAAGAAGCACGAGCGGCACCCCATCTTCGGCAACATCGTGGACGAG
GTGGCCTACCACGAGAAGTACCCCACCATCTACCACCTGAGAAAGAAACTGGTGGACAGCAC
CGACAAGGCCGACCTGCGGCTGATCTATCTGGCCCTGGCCCACATGATCAAGTTCCGGGGCC
ACTTCCTGATCGAGGGCGACCTGAACCCCGACAACAGCGACGTGGACAAGCTGTTCATCCAG
CTGGTGCAGACCTACAACCAGCTGTTCGAGGAAAACCCCATCAACGCCAGCGGCGTGGACGC
CAAGGCCATCCTGTCTGCCAGACTGAGCAAGAGCAGACGGCTGGAAAATCTGATCGCCCAGC
TGCCCGGCGAGAAGAAGAATGGCCTGTTCGGCAACCTGATTGCCCTGAGCCTGGGCCTGACC
CCCAACTTCAAGAGCAACTTCGACCTGGCCGAGGATGCCAAACTGCAGCTGAGCAAGGACAC
CTACGACGACGACCTGGACAACCTGCTGGCCCAGATCGGCGACCAGTACGCCGACCTGTTTC
TGGCCGCCAAGAACCTGTCCGACGCCATCCTGCTGAGCGACATCCTGAGAGTGAACACCGAG
ATCACCAAGGCCCCCCTGAGCGCCTCTATGATCAAGAGATACGACGAGCACCACCAGGACCT
GACCCTGCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGAGTTCTAC
AAGTTCATCAAGCCCATCCTGGAAAAGATGGACGGCACCGAGGAACTGCTCGTGAAGCTGAA
CAGAGAGGACCTGCTGCGGAAGCAGCGGACCTTCGACAACGGCAGCATCCCCCACCAGATCC
ACCTGGGAGAGCTGCACGCCATTCTGCGGCGGCAGGAAGATTTTTACCCATTCCTGAAGGAC
AACCGGGAAAAGATCGAGAAGATCCTGACCTTCCGCATCCCCTACTACGTGGGCCCTCTGGC
CAGGGGAAACAGCAGATTCGCCTGGATGACCAGAAAGAGCGAGGAAACCATCACCCCCTGGA
ACTTCGAGGAAGTGGTGGACAAGGGCGCTTCCGCCCAGAGCTTCATCGAGCGGATGACCAAC
TTCGATAAGAACCTGCCCAACGAGAAGGTGCTGCCCAAGCACAGCCTGCTGTACGAGTACTT
CACCGTGTATAACGAGCTGACCAAAGTGAAATACGTGACCGAGGGAATGAGAAAGCCCGCCT
TCCTGAGCGGCGAGCAGAAAAAGGCCATCGTGGACCTGCTGTTCAAGACCAACCGGAAAGTG
ACCGTGAAGCAGCTGAAAGAGGACTACTTCAAGAAAATCGAGTGCTTCGACTCCGTGGAAAT
CTCCGGCGTGGAAGATCGGTTCAACGCCTCCCTGGGCACATACCACGATCTGCTGAAAATTA
TCAAGGACAAGGACTTCCTGGACAATGAGGAAAACGAGGACATTCTGGAAGATATCGTGCTG
ACCCTGACACTGTTTGAGGACAGAGAGATGATCGAGGAACGGCTGAAAACCTATGCCCACCT
GTTCGACGACAAAGTGATGAAGCAGCTGAAGCGGCGGAGATACACCGGCTGGGGCAGGCTGA
GCCGGAAGCTGATCAACGGCATCCGGGACAAGCAGTCCGGCAAGACAATCCTGGATTTCCTG
AAGTCCGACGGCTTCGCCAACAGAAACTTCATGCAGCTGATCCACGACGACAGCCTGACCTT
TAAAGAGGACATCCAGAAAGCCCAGGTGTCCGGCCAGGGCGATAGCCTGCACGAGCACATTG
CCAATCTGGCCGGCAGCCCCGCCATTAAGAAGGGCATCCTGCAGACAGTGAAGGTGGTGGAC
GAGCTCGTGAAAGTGATGGGCCGGCACAAGCCCGAGAACATCGTGATCGAAATGGCCAGAGA
GAACCAGACCACCCAGAAGGGACAGAAGAACAGCCGCGAGAGAATGAAGCGGATCGAAGAGG
GCATCAAAGAGCTGGGCAGCCAGATCCTGAAAGAACACCCCGTGGAAAACACCCAGCTGCAG
AACGAGAAGCTGTACCTGTACTACCTGCAGAATGGGCGGGATATGTACGTGGACCAGGAACT
GGACATCAACCGGCTGTCCGACTACGATGTGGACCATATCGTGCCTCAGAGCTTTCTGAAGG
ACGACTCCATCGACAACAAGGTGCTGACCAGAAGCGACAAGAACCGGGGCAAGAGCGACAAC
GTGCCCTCCGAAGAGGTCGTGAAGAAGATGAAGAACTACTGGCGGCAGCTGCTGAACGCCAA
GCTGATTACCCAGAGAAAGTTCGACAATCTGACCAAGGCCGAGAGAGGCGGCCTGAGCGAAC
TGGATAAGGCCGGCTTCATCAAGAGACAGCTGGTGGAAACCCGGCAGATCACAAAGCACGTG
GCACAGATCCTGGACTCCCGGATGAACACTAAGTACGACGAGAATGACAAGCTGATCCGGGA
AGTGAAAGTGATCACCCTGAAGTCCAAGCTGGTGTCCGATTTCCGGAAGGATTTCCAGTTTT
ACAAAGTGCGCGAGATCAACAACTACCACCACGCCCACGACGCCTACCTGAACGCCGTCGTG
GGAACCGCCCTGATCAAAAAGTACCCTAAGCTGGAAAGCGAGTTCGTGTACGGCGACTACAA
GGTGTACGACGTGCGGAAGATGATCGCCAAGAGCGAGCAGGAAATCGGCAAGGCTACCGCCA
AGTACTTCTTCTACAGCAACATCATGAACTTTTTCAAGACCGAGATTACCCTGGCCAACGGC
GAGATCCGGAAGCGGCCTCTGATCGAGACAAACGGCGAAACCGGGGAGATCGTGTGGGATAA
GGGCCGGGATTTTGCCACCGTGCGGAAAGTGCTGAGCATGCCCCAAGTGAATATCGTGAAAA
AGACCGAGGTGCAGACAGGCGGCTTCAGCAAAGAGTCTATCCTGCCCAAGAGGAACAGCGAT
AAGCTGATCGCCAGAAAGAAGGACTGGGACCCTAAGAAGTACGGCGGCTTCGACAGCCCCAC
CGTGGCCTATTCTGTGCTGGTGGTGGCCAAAGTGGAAAAGGGCAAGTCCAAGAAACTGAAGA
GTGTGAAAGAGCTGCTGGGGATCACCATCATGGAAAGAAGCAGCTTCGAGAAGAATCCCATC
GACTTTCTGGAAGCCAAGGGCTACAAAGAAGTGAAAAAGGACCTGATCATCAAGCTGCCTAA
GTACTCCCTGTTCGAGCTGGAAAACGGCCGGAAGAGAATGCTGGCCTCTGCCGGCGAACTGC
AGAAGGGAAACGAACTGGCCCTGCCCTCCAAATATGTGAACTTCCTGTACCTGGCCAGCCAC
TATGAGAAGCTGAAGGGCTCCCCCGAGGATAATGAGCAGAAACAGCTGTTTGTGGAACAGCA
CAAGCACTACCTGGACGAGATCATCGAGCAGATCAGCGAGTTCTCCAAGAGAGTGATCCTGG
CCGACGCTAATCTGGACAAAGTGCTGTCCGCCTACAACAAGCACCGGGATAAGCCCATCAGA
GAGCAGGCCGAGAATATCATCCACCTGTTTACCCTGACCAATCTGGGAGCCCCTGCCGCCTT
CAAGTACTTTGACACCACCATCGACCGGAAGAGGTACACCAGCACCAAAGAGGTGCTGGACG
CCACCCTGATCCACCAGAGCATCACCGGCCTGTACGAGACACGGATCGACCTGTCTCAGCTG
GGAGGCGAC.

In some embodiments, the construct comprising a promoter and a nuclease further comprises at least two inverted terminal repeat (ITR) sequences. In some embodiments, the construct comprising a promoter and a nuclease further comprises at least two ITR sequences from isolated or derived from an AAV of serotype 2 (AAV2). In some embodiments, the construct comprising a promoter and a nuclease further comprises at least two ITR sequences each comprising or consisting of a nucleotide sequence of GGCCACTCCCTCTCTGCGCGCTCGCTCGCTCACTGAGGCCGGGCGACCAAAGGTCGCCCGAC GCCCGGGCTTTGCCCGGGCGGCCTCAGTGAGCGAGCGAGCGCGCAGAGAGGGA (SEQ ID NO: 71). In some embodiments, the construct comprising a promoter and a nuclease further comprises at least two ITR sequences, wherein the first ITR sequence comprises or consists of a nucleotide sequence of CCTGCAGGCAGCTGCGCGCTCGCTCGCTCACTGAGGCCGCCCGGGCAAAGCCCGGGCGTCGG GCGACCTTTGGTCGCCCGGCCTCAGTGAGCGAGCGAGCGCGCAGAGAGGGAGTGGCCAACTC CATCACTAGGGGTTCCT (SEQ ID NO: 72) and the second ITR sequence comprises or consist of a nucleotide sequence of AGGAACCCCTAGTGATGGAGTTGGCCACTCCCTCTCTGCGCGCTCGCTCGCTCACTGAGGCC GGGCGACCAAAGGTCGCCCGACGCCCGGGCTTTGCCCGGGCGGCCTCAGTGAGCGAGCGAGC GCGCAGCTGCCTGCAGG (SEQ ID NO: 73). In some embodiments, the construct comprises or consists of, from 5′ to 3′ a first ITR, a sequence encoding a promoter, a sequence encoding a nuclease and a second ITR. In some embodiments, the construct comprises or consists of, from 5′ to 3′ a first AAV2 ITR, a sequence encoding an cTnT promoter, a sequence encoding a SpCas9 nuclease and a second AAV2 ITR. In some embodiments, the construct comprising or consisting of, from 5′ to 3′ a first ITR, a sequence encoding a promoter, a sequence encoding a nuclease and a second ITR, further comprises a poly A sequence. In some embodiments, the polyA sequence comprises or consists of a minipolyA sequence. Exemplary minipolyA sequences of the disclosure comprise or consist of a nucleotide sequence of TAGCAATAAAGGATCGTTTATTTTCATTGGAAGCGTGTGTTGGTTTTTTGATCAGGCGCG (SEQ ID NO: 74). In some embodiments, the construct comprises or consists of, from 5′ to 3′ a first ITR, a sequence encoding a promoter, a sequence encoding a nuclease, a poly A sequence and a second ITR. In some embodiments, the construct comprises or consists of, from 5′ to 3′ a first ITR, a sequence encoding a promoter, a sequence encoding a nuclease, a minipoly A sequence and a second ITR. In some embodiments, the construct comprises or consists of, from 5′ to 3′ a first AAV2 ITR, a sequence encoding an cTnT promoter, a sequence encoding a SpCas9 nuclease, a minipoly A sequence and a second AAV2 ITR. In some embodiments, the construct comprising, from 5′ to 3′ a first ITR, a sequence encoding a promoter, a sequence encoding a nuclease, a poly A sequence and a second ITR, further comprises at least one nuclear localization signal. In some embodiments, the construct comprising, from 5′ to 3′ a first ITR, a sequence encoding a promoter, a sequence encoding a nuclease, a poly A sequence and a second ITR, further comprises at least two nuclear localization signals. Exemplary nuclear localization signals of the disclosure comprise or consist of a nucleotide sequence of AAGCGTCCTGCTGCTACTAAGAAAGCTGGTCAAGCTAAGAAAAAGAAA (SEQ ID NO: 75) or a nucleotide sequence of ATGGCCCCAAAGAAGAAGCGGAAGGTCGGTATCCACGGAGTCCCAGCAGCC (SEQ ID NO: 76). In some embodiments, the construct comprises or consists of, from 5′ to 3′ a first ITR, a sequence encoding a promoter, a sequence encoding a first nuclear localization signal, a sequence encoding a nuclease, a poly A sequence and a second ITR. In some embodiments, the construct comprises or consists of, from 5′ to 3′ a first ITR, a sequence encoding a promoter, a sequence encoding a first nuclear localization signal, a sequence encoding a nuclease, a sequence encoding a second nuclear localization signal, a poly A sequence and a second ITR. In some embodiments, the construct comprising, from 5′ to 3′ a first ITR, a sequence encoding a promoter, a sequence encoding a first nuclear localization signal, a sequence encoding a nuclease, a sequence encoding a second nuclear localization signal, a poly A sequence and a second ITR, further comprises a stop codon. The stop codon may have a sequence of TAG, TAA, or TGA. In some embodiments, the construct comprises or consists of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR. In some embodiments, the construct comprising or consisting of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR, further comprises transposable element inverted repeats. Exemplary transposable element inverted repeats of the disclosure comprise or consist of a nucleotide sequence of TGTGGGCGGACAAAATAGTTGGGAACTGGGAGGGGTGGAAATGGAGTTTTTAAGGATTATTT AGGGAAGAGTGACAAAATAGATGGGAACTGGGTGTAGCGTCGTAAGCTAATACGAAAATTAA AAATGACAAAATAGTTTGGAACTAGATTTCACTTATCTGGTT (SEQ ID NO: 77) and/or a nucleotide sequence of GAATATAGTCTTTACCATGCCCTTGGCCACGCCCCTCTTTAATACGACGGGCAATTTGCACT TCAGAAAATGAAGAGTTTGCTTTAGCCATAACAAAAGTCCAGTATGCTTTTTCACAGCATAA CTGGACTGATTTCAGTTTACAACTATTCTGTCTAGTTTAAGACTTTATTGTCATAGTTTAGA TCTATTTTGTTCAGTTTAAGACTTTATTGTCCGCCCACA (SEQ ID NO: 78). In some embodiments, the construct comprises or consists of, from 5′ to 3′ a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, and a second transposable element inverted repeat. In some embodiments, the construct comprising or consisting of, from 5′ to 3′, a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, and a second transposable element inverted repeat, further comprises a regulatory sequence. Exemplary regulatory sequences of the disclosure comprise or consist of a nucleotide sequence of CATGCAAGCTGTAGCCAACCACTAGAACTATAGCTAGAGTCCTGGGCGAACAAACGATGCTC GCCTTCCAGAAAACCGAGGATGCGAACCACTTCATCCGGGGTCAGCACCACCGGCAAGCGCC GCGACGGCCGAGGTCTTCCGATCTCCTGAAGCCAGGGCAGATCCGTGCACAGCACCTTGCCG TAGAAGAACAGCAAGGCCGCCAATGCCTGACGATGCGTGGAGACCGAAACCTTGCGCTCGTT CGCCAGCCAGGACAGAAATGCCTCGACTTCGCTGCTGCCCAAGGTTGCCGGGTGACGCACAC CGTGGAAACGGATGAAGGCACGAACCCAGTTGACATAAGCCTGTTCGGTTCGTAAACTGTAA TGCAAGTAGCGTATGCGCTCACGCAACTGGTCCAGAACCTTGACCGAACGCAGCGGTGGTAA CGGCGCAGTGGCGGTTTTCATGGCTTGTTATGACTGTTTTTTTGTACAGTCTATGCCTCGGG CATCCAAGCAGCAAGCGCGTTACGCCGTGGGTCGATGTTTGATGTTATGGAGCAGCAACGAT GTTACGCAGCAGCAACGATGTTACGCAGCAGGGCAGTCGCCCTAAAACAAAGTTAGGTGGCT CAAGTATGGGCATCATTCGCACATGTAGGCTCGGCCCTGACCAAGTCAAATCCATGCGGGCT GCTCTTGATCTTTTCGGTCGTGAGTTCGGAGACGTAGCCACCTACTCCCAACATCAGCCGGA CTCCGATTACCTCGGGAACTTGCTCCGTAGTAAGACATTCATCGCGCTTGCTGCCTTCGACC AAGAAGCGGTTGTTGGCGCTCTCGCGGCTTACGTTCTGCCCAAGTTTGAGCAGCCGCGTAGT GAGATCTATATCTATGATCTCGCAGTCTCCGGCGAGCACCGGAGGCAGGGCATTGCCACCGC GCTCATCAATCTCCTCAAGCATGAGGCCAACGCGCTTGGTGCTTATGTGATCTACGTGCAAG CAGATTACGGTGACGATCCCGCAGTGGCTCTCTATACAAAGTTGGGCATACGGGAAGAAGTG ATGCACTTTGATATCGACCCAAGTACCGCCACCTAACAATTCGTTCAAGCCGAGATCGGCTT CCCGGCCGCGGAGTTGTTCGGTAAATTGTCACAACGCCG (SEQ ID NO: 79). In some embodiments, the construct comprises or consists of, from 5′ to 3′ a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, a regulatory sequence and a second transposable element inverted repeat. In some embodiments, the construct may further comprise one or more spacer sequences. Exemplary spacer sequences of the disclosure have length from 1-1500 nucleotides, inclusive of all ranges therebetween. In some embodiments, the spacer sequences may be located either 5′ to or 3′ to an ITR, a promoter, a nuclear localization sequence, a nuclease, a stop codon, a polyA sequence, a transposable element inverted repeat, and/or a regulator element.

### F. AAV-sgRNA Vectors

In some embodiments, at least a first sequence encoding a gRNA and a second sequence encoding a gRNA may be packaged into an AAV vector. In some embodiments, at least a first sequence encoding a gRNA, a second sequence encoding a gRNA, and a third sequence encoding a gRNA may be packaged into an AAV vector. In some embodiments, at least a first sequence encoding a gRNA, a second sequence encoding a gRNA, a third sequence encoding a gRNA, and a fourth sequence encoding a gRNA may be packaged into an AAV vector. In some embodiments, at least a first sequence encoding a gRNA, a second sequence encoding a gRNA, a third sequence encoding a gRNA, a fourth sequence encoding a gRNA, and a fifth sequence encoding a gRNA may be packaged into an AAV vector. In some embodiments, a plurality of sequences encoding a gRNA are packaged into an AAV vector. For example, 1, 2, 3, 4, 5, 6, 7, 8, 9, 10, 11, 12, 13, 14, 15, 16, 17, 18, 19, or 20 sequences encoding a gRNA may be packaged into an AAV vector. In some embodiments, each sequence encoding a gRNA is different. In some embodiments, at least 1, 2, 3, 4, 5, 6, 7, 8, 9, 10, 11, 12, 13, 14, 15, 16, 17, 18, 19, or 20 of the sequences encoding a gRNA are the same. In some embodiments, all of the sequence encoding a gRNA are the same.

In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11 or any combination thereof.

Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs are isolated or derived from an AAV vector of a first serotype and a sequence encoding a capsid protein of the AAV-sgRNA vector is isolated or derived from an AAV vector of a second serotype. In some embodiments, the first serotype and the second serotype are the same. In some embodiments, the first serotype and the second serotype are not the same. In some embodiments, the first serotype is AAV1, AAV2, AAV3, AAV4, AAV5, AAV6, AAV7, AAV8, AAV9, AAV10, or AAV11. In some embodiments, the second serotype is AAV1, AAV2, AAV3, AAV4, AAV5, AAV6, AAV7, AAV8, AAV9, AAV10, or AAV11. In some embodiments, the first serotype is AAV2 and the second serotype is AAV1, AAV2, AAV3, AAV4, AAV5, AAV6, AAV7, AAV8, AAV9, AAV10, or AAV11. In some embodiments, the first serotype is AAV2 and the second serotype is AAV9.

Exemplary AAV-sgRNA vectors contain two ITR (inverted terminal repeat) sequences which flank a central sequence region comprising the gRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, a first ITR is isolated or derived from an AAV vector of a first serotype, a second ITR is isolated or derived from an AAV vector of a second serotype and a sequence encoding a capsid protein of the AAV-sgRNA vector is isolated or derived from an AAV vector of a third serotype. In some embodiments, the first serotype and the second serotype are the same. In some embodiments, the first serotype and the second serotype are not the same. In some embodiments, the first serotype, the second serotype, and the third serotype are the same. In some embodiments, the first serotype, the second serotype, and the third serotype are not the same. In some embodiments, the first serotype is AAV1, AAV2, AAV3, AAV4, AAV5, AAV6, AAV7, AAV8, AAV9, AAV10, or AAV11. In some embodiments, the second serotype is AAV1, AAV2, AAV3, AAV4, AAV5, AAV6, AAV7, AAV8, AAV9, AAV10, or AAV11. In some embodiments, the third serotype is AAV1, AAV2, AAV3, AAV4, AAV5, AAV6, AAV7, AAV8, AAV9, AAV10, or AAV11. In some embodiments, the first serotype is AAV2, the second serotype is AAV4 and the third serotype is AAV1, AAV2, AAV3, AAV4, AAV5, AAV6, AAV7, AAV8, AAV9, AAV10, or AAV11. In some embodiments, the first serotype is AAV2, the second serotype is AAV4 and the third serotype is AAV9. Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-sgRNA vector may comprise additional elements to facilitate packaging of the vector and expression of the sgRNA. In some embodiments, the AAV-sgRNA vector may comprise a transposable element. In some embodiments, the AAV-sgRNA vector may comprise a regulatory element. In some embodiments, the regulatory element comprises an activator or a repressor. In some embodiments, the AAV-sgRNA sequence may comprise a non-functional or “stuffer” sequence. Exemplary stuffer sequences of the disclosure may have some (a non-zero percentage of) identity or homology to a genomic sequence of a mammal (including a human). Alternatively, exemplary stuffer sequences of the disclosure may have no identify or homology to a genomic sequence of a mammal (including a human). Exemplary stuffer sequences of the disclosure may comprise or consist of naturally occurring non-coding sequences or sequences that are neither transcribed nor translated following administration of the AAV vector to a subject.

In some embodiments, the AAV-sgRNA vector may be optimized for production in yeast, bacteria, insect cells, or mammalian cells. In some embodiments, the AAV-sgRNA vector may be optimized for expression in human cells. In some embodiments, the AAV-Cas9 vector may be optimized for expression in a baculovirus expression system.

In some embodiments, the AAV-sgRNA vector comprises at least one promoter. In some embodiments, the AAV-sgRNA vector comprises at least two promoters. In some embodiments, the AAV-sgRNA vector comprises at least three promoters. In some embodiments, the AAV-sgRNA vector comprises at least four promoters. In some embodiments, the AAV-sgRNA vector comprises at least five promoters. Exemplary promoters include, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus. Further exemplary promoters include the U6 promoter, the H1 promoter, and the 7SK promoter.

In some embodiments, the AAV vector comprises a first sequence encoding a gRNA and a second sequence encoding a gRNA, a first promoter drives expression of the first sequence encoding a gRNA and a second promoter drives expression of the second sequence encoding a gRNA. In some embodiments, the first and second promoters are the same. In some embodiments, the first and second promoters are different. In some embodiments, the first and second promoters are selected from the H1 promoter, the U6 promoter, and the 7SK promoter. In some embodiments, the first sequence encoding a gRNA and the second sequence encoding a gRNA are identical. In some embodiments, the first sequence encoding a gRNA and the second sequence encoding a gRNA are not identical.

In some embodiments, the AAV vector comprises a first sequence encoding a gRNA, a second sequence encoding a gRNA, and a third sequence encoding a gRNA, a first promoter drives expression of the first sequence encoding a gRNA, a second promoter drives expression of the second sequence encoding a gRNA, and a third promoter drives expression of a third sequence encoding a gRNA. In some embodiments, at least two of the first, second, and third promoters are the same. In some embodiments, each of the first, second, and third promoters are different. In some embodiments, the first, second, and third promoters are selected from the H1 promoter, the U6 promoter, and the 7SK promoter. In some embodiments, the first promoter is the U6 promoter. In some embodiments, the second promoter is the H1 promoter. In some embodiments, the third promoter is the 7SK promoter. In some embodiments, the first promoter is the U6 promoter, the second promoter is the H1 promoter, and the third promoter is the 7SK promoter. In some embodiments, the first sequence encoding a gRNA, the second sequence encoding a gRNA, and the third sequence encoding a gRNA are identical. In some embodiments, the first sequence encoding a gRNA, the second sequence encoding a gRNA, and the third sequence encoding a gRNA are not identical.

In some embodiments, the AAV vector comprises a first sequence encoding a gRNA, a second sequence encoding a gRNA, a third sequence encoding a gRNA, and a fourth sequence encoding a gRNA, a first promoter drives expression of the first sequence encoding a gRNA, a second promoter drives expression of the second sequence encoding a gRNA, a third promoter drives expression of the third sequence encoding a gRNA, and a fourth promoter drives expression of the fourth sequence encoding a gRNA. In some embodiments, at least two of the first, second, third, and fourth promoters are the same. In some embodiments, each of the first, second, third, and fourth promoters are different. In some embodiments, each of the first, second, third and fourth promoters are selected from the H1 promoter, the U6 promoter, and the 7SK promoter. In some embodiments, the first sequence encoding a gRNA, the second sequence encoding a gRNA, the third sequence encoding a gRNA, and the fourth sequence encoding a gRNA are identical. In some embodiments, the first sequence encoding a gRNA, the second sequence encoding a gRNA, the third sequence encoding a gRNA, and the fourth sequence encoding a gRNA are not identical.

In some embodiments, the AAV vector comprises a first sequence encoding a gRNA, a second sequence encoding a gRNA, a third sequence encoding a gRNA, a fourth sequence encoding a gRNA, and a fifth sequence encoding a gRNA, a first promoter drives expression of the first sequence encoding a gRNA, a second promoter drives expression of the second sequence encoding a gRNA, a third promoter drives expression of the third sequence encoding a gRNA, a fourth promoter drives expression of the fourth sequence encoding a gRNA, and a fifth promoter drives expression of the fifth sequence encoding a gRNA. In some embodiments, at least two of the first, second, third, fourth, and fifth promoters are the same. In some embodiments, each of the first, second, third, fourth, and fifth promoters are different. In some embodiments, each of the first, second, third, and fourth promoters are different. In some embodiments, each of the first, second, third, fourth and fifth promoters are selected from the H1 promoter, the U6 promoter, and the 7SK promoter. In some embodiments, the first sequence encoding a gRNA, the second sequence encoding a gRNA, the third sequence encoding a gRNA, the fourth sequence encoding a gRNA, and the fifth sequence encoding a gRNA are identical. In some embodiments, the first sequence encoding a gRNA, the second sequence encoding a gRNA, the third sequence encoding a gRNA, the fourth sequence encoding a gRNA, and the fifth sequence encoding a gRNA are not identical.

### IV. Pharmaceutical Compositions and Delivery Methods

Any of the AAV viral particles, AAV vectors, polynucleotides, or vectors encoding polynucleotides disclosed herein may be formulated into a pharmaceutical composition. In some embodiments, pharmaceutical composition may further include one ore more pharmaceutically acceptable carriers, diluents or excipients. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In some embodiments, the pharmaceutical compositions or formulations can be for administration by subcutaneous, intramuscular, intravenous, intraperitoneal, intracardiac, intraarticular, or intracavernous injection. In some embodiments, the pharmaceutical compositions or formulations are for parenteral administration, such as intravenous, intracerebroventricular injection, intra-cisterna magna injection, intra-parenchymal injection, intraperitoneal, intracardiac, intraarticular, or intracavernous injection or a combination thereof. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating AAV particles in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The pharmaceutical compositions disclosed herein may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

For clinical applications, pharmaceutical compositions are prepared in a form appropriate for the intended application. Generally, this entails preparing compositions that are essentially free of pyrogens, as well as other impurities that could be harmful to humans or animals.

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients of the present disclosure, its use in therapeutic compositions may be used. Supplementary active ingredients also can be incorporated into the compositions, provided they do not inactivate the vectors or cells of the compositions.

In some embodiments, the active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine) and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the nucleotide editing Cas9 and gRNAs described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding nucleotide editing Cas9 and a guide RNA that targets a dystrophin splice site are provided to a cell ex vivo before the cell is introduced or reintroduced to a patient.

In various embodiments, compositions disclosed herein may be effective for treating heart disease following administration to a subject in need. In other embodiments, compositions disclosed herein may be effective for treating one or more cardiomyopathies following administration to a subject in need. In still other embodiments, compositions disclosed herein may be effective for treating DCM following administration to a subject in need. In other embodiments, compositions disclosed herein may be effective for improving at least one symptom of DCM following administration to a subject in need.

A suitable subject herein includes a human, a livestock animal, a companion animal, a lab animal, or a zoological animal. In some embodiments, the subject may be a rodent, e.g., a mouse, a rat, a guinea pig, etc. In some embodiments, the subject may be a livestock animal. Non-limiting examples of suitable livestock animals may include pigs, cows, horses, goats, sheep, llamas and alpacas. In some embodiments, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In various embodiments, a subject in need may have been diagnosed with at least one heart disease. In some aspects, the subject may have one or more cardiomyopathies. In some embodiments, the subject may have DCM. In some embodiments, a subject may at least one symptom of DCM. In some aspects, a symptom of DCM can be fatigue. In some embodiments, a symptom of DCM can be dyspnea. In some embodiments, a symptom of DCM can be edema. In some embodiments, a symptom of DCM can be ascites. In some embodiments, a symptom of DCM can be chest pain. In still other aspects, a symptom of DCM can be a heart murmur.

In some embodiments, methods of administering compositions disclosed herein may decrease and/or reverse cardiomyopathy-induced cardiac fibrosis compared to cardiomyopathy-induced cardiac fibrosis in an untreated subject with identical disease condition and predicted outcome. In some embodiments, methods of administering compositions disclosed herein may decrease and/or reverse cardiomyopathy-induced left ventricle dilation compared to cardiomyopathy-induced left ventricle dilation in an untreated subject with identical disease condition and predicted outcome.

Other embodiments of the present disclosure are methods of administering compositions disclosed herein to a subject in need wherein administration treats cardiomyopathy (e.g., DCM). Still other embodiments of the present disclosure are methods of administering compositions disclosed herein to a subject in need wherein at least one symptom of cardiomyopathy (e.g., DCM) is improved by at least 25% within one month after administration.

In various embodiments, compositions disclosed herein may be administered by parenteral administration. As used herein, “by parenteral administration” refers to administration of the compositions disclosed herein via a route other than through the digestive tract. In some embodiments, compositions disclosed herein may be administered by parenteral injection. In some aspects, administration of the disclosed compositions by parenteral injection may be by subcutaneous, intramuscular, intravenous, intraperitoneal, intracardiac, intraarticular, or intracavernous injection. In some embodiments, administration of the disclosed compositions by parenteral injection may be by slow or bolus methods as known in the field. In some embodiments, the route of administration by parenteral injection can be determined by the target location. In some embodiments, compositions disclosed herein may be formulated for parenteral administration by intracardiac injection. In some embodiments, compositions disclosed herein may be formulated for parenteral administration by catheter-based intracoronary infusion. In some embodiments, compositions disclosed herein may formulated for parenteral administration by pericardial injection.

In various embodiments, the dose of compositions disclosed herein to be administered are not particularly limited, and may be appropriately chosen depending on conditions such as a purpose of preventive and/or therapeutic treatment, a type of a disease, the body weight or age of a subject, severity of a disease and the like. In some embodiments, administration of a dose of a composition disclosed herein may comprise a therapeutically effective amount of the composition disclosed herein. As used herein, the term “therapeutically effective” refers to an amount of administered composition that treats heart disease, reduces presentation of at least one symptom associated with heart disease, reverses/prevents cardio fibrosis, reverse/prevent dilation of at least one heart ventricle, reduces total heart weight, improved heart function, increases survivability, or a combination thereof.

In some embodiments, a composition disclosed herein may be administered to a subject in need thereof once. In some embodiments, a composition disclosed herein may be administered to a subject in need thereof more than once. In some embodiments, a first administration of a composition disclosed herein may be followed by a second administration of a composition disclosed herein. In some embodiments, a first administration of a composition disclosed herein may be followed by a second and third administration of a composition disclosed herein. In some embodiments, a first administration of a composition disclosed herein may be followed by a second, third, and fourth administration of a composition disclosed herein. In some embodiments, a first administration of a composition disclosed herein may be followed by a second, third, fourth, and fifth administration of a composition disclosed herein.

The number of times a composition may be administered to an subject in need thereof can depend on the discretion of a medical professional, the severity of the heart disease, and the subject's response to the formulation. In some embodiments, a composition disclosed herein may be administered continuously; alternatively, the dose of drug being administered may be temporarily reduced or temporarily suspended for a certain length of time (i.e., a “drug holiday”). In some aspects, the length of the drug holiday can vary between 2 days and 1 year, including by way of example only, 2 days, 1 week, 1 month, 6 months, and 1 year. In another aspect, dose reduction during a drug holiday may be from 10%-100%, including by way of example only 10%, 25%, 50%, 75%, and 100%.

In various embodiments, the desired daily dose of compositions disclosed herein may be presented in a single dose or as divided doses administered simultaneously (or over a short period of time) or at appropriate intervals. In other embodiments, administration of a composition disclosed herein may be administered to a subject about once a day, about twice a day, about three times a day. In still other embodiments, administration of a composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter. In a preferred embodiment, administration of a composition disclosed herein may be administered to a subject once about 4 weeks.

In some embodiments, a composition as disclosed may be initially administered followed by a subsequent administration of one for more different compositions or treatment regimens. In other embodiments, a composition as disclosed may be administered after administration of one for more different compositions or treatment regimens.

### V. Kits

Some embodiments of the present disclosure include kits for packaging and transporting CRISPR/Cas9 systems and/or novel gRNAs disclosed herein or known gRNAs disclosed herein and further include at least one container.

In some embodiments, the kit can additionally comprise instructions for use of CRISPR/Cas9 systems, gRNAs, and or AAV particles in any of the methods described herein. The included instructions may comprise a description of administration of pharmaceutical compositions as disclosed herein to a subject to achieve the intended activity in a subject. The kit may further comprise a description of selecting a subject suitable for treatment based on identifying whether the subject is in need of the treatment. In some embodiments, the instructions may comprise a description of administering pharmaceutical compositions disclosed herein to a subject who has or is suspected of having a cardiomyopathy.

### VI. Definitions

The term “nucleotide editing Cas9” refers to a Cas9 protein fused to a base editor or a prime editor. Non-limiting examples of Cas9 include SpCas9, SpCas9-NG, SaCas9, SaCas9-KKH, SauCas9, and SlugCas9. Non limiting examples of abase editor include ABEmax, ABE8e, ABE8eV106W, ABE8.20-m.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polynucleotide,” “nucleic acid” and “transgene” are also used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

As used herein, “AAV” refers to an adeno-associated virus vector. As used herein, “AAV” refers to any AAV serotype and variant, including but not limited to an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), AAV9 vector, AAV9P vector (also known as AAVMYO, see, Weinmann et al., 2020, Nature Communications, 11:5432), and Myo-AAV vectors described in Tabebordbar et al., 2021, Cell, 184:1-20 (e.g., MyoAAV 1A, 2A, 3A, 4A, 4C, or 4E), wherein the number following AAV indicates the AAV serotype. The term “AAV” can also refer to any known AAV (vector) system. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. Structurally, AAVs are small (25 nm), single-DNA stranded non-enveloped viruses with an icosahedral capsid. Naturally occurring or engineered AAV serotypes and variants that differ in the composition and structure of their capsid protein have varying tropism, i.e., ability to transduce different cell types. When combined with active promoters, this tropism defines the site of gene expression.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

Target sequences for Cas9s include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′) and may be capable of functioning even when positioned either upstream or downstream of the promoter.

Promoters and/or enhancers may be derived in their entirety from a native gene or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. For clarity, the terms “spacer sequence”, “spacer,” “protospacer,” “guide sequence,” or “targeting sequence” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the disclosure, such polypeptides encoded by nucleic acid sequences are not required to be identical to the endogenous protein that is defective, or whose activity, function, or expression is insufficient, deficient or absent in a treated mammal.

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors or nanoparticles to various organs and tissues.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the disclosure will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder. The terms “treat” and “treatment” also refer to both therapeutic treatment and prophylactic or preventative measures, wherein the object is to prevent, inhibit, reduce, or decrease an undesired physiological change or disorder, such as the development, progression or worsening of the disorder. For purposes of this disclosure, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

As used herein the specification, “a” or “an” may mean one or more. As used herein in the claim(s), when used in conjunction with the word “comprising,” the words “a” or “an” may mean one or more than one.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

The use of the term “or” in the claims is used to mean “and/or” unless explicitly indicated to refer to alternatives only or the alternatives are mutually exclusive, although the disclosure supports a definition that refers to only alternatives and “and/or.” Furthermore, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive. As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value. For example, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

It should also be understood that, unless clearly indicated to the contrary, in any methods claimed herein that include more than one step or act, the order of the steps or acts of the method is not necessarily limited to the order in which the steps or acts of the method are recited.

As the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Study design. This study was aimed to test the therapeutic potential of base editing (BE) and prime editing (PE) for RNA binding motif protein 20 gene (RBM20) mutations in human cells and a mouse model of dilated cardiomyopathy (DCM). Male mice were used for all experiments. All echocardiography experiments were performed and analyzed by a single blinded operator. Each experiment was performed in replicate as indicated by n values in the figure legends.

Study approval. All experimental procedures involving animals in this study were reviewed and approved by the University of Texas Southwestern Medical Center's Institutional Animal Care and Use Committee. Use of induce pluripotent stem cell lines was reviewed and approved by the University of Texas Southwestern Medical Center's Stem Cell Research Oversight Committee.

All-In-One SpCas9-ABE variants and SaCas9-ABE vectors. All variants of SpCas9 and SaCas9 were synthesized by g-Block (Integrated DNA Technologies), and subcloned into AgeI/FseI digested pSpCas9(BB)-2A-GFP (px458) (Addgene plasmid #48138) (25) from Feng Zhang's lab (Broad Institute) using In-Fusion ligation (Takara Bio) according to the manufacturer's protocol. These vectors were digested by AgeI and ApaI. The inserts were transcribed from NG-ABEmax (Addgene plasmid #124163) (26) and NG-ABE8e (Addgene plasmid #138491) (20) from David Liu's lab (Broad Institute). These inserts were subcloned into pre-digested pSpCas9-NG-2A-GFP, pSpCas9-VRQR-2A-GFP and pSaCas9-2A-GFP using In-Fusion cloning kit (Takara Bio) according to the manufacturer's protocol. The sgRNAs for adenine base editing (ABE) of the RBM20^{R634Q} mutation were subcloned into engineered vectors using BbsI and T4 ligation. Primers are listed in Table 1.

Human iPSC culture and generation of isogenic lines. Human iPSC culturing was performed as previously described (27). Briefly, iPSCs were maintained in mTeSR plus medium (STEMCELL Technologies) and passaged using Versene (Thermo Fisher) and 10 μM Rock inhibitor, Y-27632 (Selleckchem) every four days. A single-cell suspension of 8×10⁵ iPSCs was mixed with a single-stranded oligodeoxynucleotide (ssODN) template and 5 μg of pSpCas9(BB)-2A-GFP (px458) plasmid containing sgRNA for exon 9 of RBM20. The mixture was transfected by Primary Cell 4D-Nucleofector X kit (Lonza) according to the manufacturer's protocol. After nucleofection, iPSCs were maintained in mTeSR plus medium with Rock inhibitor and Primocin (Invivogen). GFP⁺ cells were sorted by FACS at 48 hr after nucleofection and expanded. GFP⁺ single iPSCs were picked and genomic sequencing was performed.

BE and PE in iPSCs. For ABE, 5 μg of engineered All-In-One vector including sgRNA were transfected into heterozygous (R634Q/+) or homozygous (R634Q/R634Q) iPSCs by nucleofection. After 48 hr, GFP⁺ cells were sorted by FACS and expanded. For PE, pegRNA and epegRNA were subcloned into pU6-pegRNA-GG-acceptor plasmid (Addgene plasmid #132777) (22) from David Liu's lab. The nicking sgRNA was subcloned into pmCherry_gRNA plasmid (Addgene plasmid #80457) from Ervin Welker's lab (Hungarian Academy of Sciences). For the PE3b system, pCMV-PE2-P2A-GFP (Addgene plasmid #132776) (22) from David Liu's lab, pegRNA and nicking sgRNA plasmids (4.5 μg, 1.5 μg and 0.75 μg, respectively) were transfected into 8×10⁵ homozygous (R636S/R636S) iPSCs by nucleofection. For PE3bmax coupled with epegRNA, pCMV-PEmax (Addgene plasmid #174820) (24) from David Liu's lab, epegRNA and nicking sgRNA plasmids (4.5 μg, 1.5 μg and 0.75 μg, respectively) were transfected by nucleofection. After 48 hr, GFP+ and mCherry+ cells in PE3b treated iPSCs, and mCherry+ cells in PE3bmax treated iPSCs were sorted by FACS and expanded. After extracting DNA, the region of exon9 in RBM20 was amplified by PCR and PCR products were subjected to ExoSAP-IT (Thermo Fisher) according to the manufacturer's protocol and sequenced.

Human iPSC cardiomyocyte differentiation. iPSCs were induced to differentiate into cardiomyocytes (CMs) using previously described methods (28). iPSCs were treated with CHIR 99021 (Selleckchem) in RPMI 1640 (Thermo Fisher Scientific) supplemented with CDM3 for 2 days (days 1 to 2). The medium was changed to RPMI supplemented with WNT-C59 (Selleckchem) for 2 days (days 3 to 4). iPSC derived CMs (iPSC-CMs) were maintained in RPMI 1640 supplemented with B27-supplement (Thermo Fisher Scientific). iPSC-CMs were purified by metabolic selection in RPMI 1640 without glucose (Thermo Fisher Scientific), supplemented with 5 mM sodium DL-lactate and CDM3 supplement for 6 days (days 10-16). After metabolic selection, iPSC-CMs were replated into 6 well plates using Tryple Express (Thermo Fisher Scientific). CMs were used for experiments on days 35-40 post differentiation. Regarding the ABE- and PE-corrected iPSC-CMs, single clones were isolated and differentiated into iPSC-CMs for assays.

Immunocytochemistry of iPSC-derived CMs. Immunocytochemistry of iPSC-CMs was performed as previously described (29). Briefly, iPSC-CMs were replaced on 12-mm cover slips coated with poly-D-lysine. After fixation with 4% PFA (15 min) and permeabilization with 0.3% Triton-X (15 min), coverslips were blocked for 1 hour with 5% goat serum/phosphate-buffered saline. Rabbit anti-RBM20 antibody (Novus Biologicals, NBP2-34038, 1:250) and mouse anti-alpha-Actinin (Sigma-Aldrich, A7811, 1:800) in 5% goat serum/phosphate-buffered saline was applied and incubated overnight at 4° C. Then, coverslips were incubated with fluorescein-conjugated goat anti-rabbit Alexa Fluor 488 and anti-mouse IgG Alexa Fluor 555 (Invitrogen). Images were taken by a Zeiss LSM-800 microscope using a 20× objective and N-SIM S Super Resolution Microscope (Nikon) using a 100× oil objective.

Immunohistochemistry. Hearts were isolated and fixed in 4% paraformaldehyde in phosphate-buffered saline for 48 hr. After fixation, images of gross hearts were taken by a Zeiss AxioZoom.V16 system. Hearts were embedded in paraffin and sectioned. Hematoxylin and eosin (H&E) and Masson's trichrome staining was performed. Images were taken by Digital Microscope (Keyence) with 4× and 40× objective magnifications. For immunohistochemistry, sections were deparaffinized and subjected to antigen retrieval using epitope retrieval solution (IHC WORLD) according to the manufacturer's protocol. Cardiomyocytes were stained by primary antibodies of cardiac troponin T (Thermo scientific, 1:200) and RBM20 (a gifted from Dr. Wei Guo, 1:400). Sections were incubated with DAPI, fluorescein-conjugated goat anti-rabbit Alexa Fluor 488 and anti-mouse IgG Alexa Fluor 555 (Invitrogen). Images were taken by a N-SIM S Super Resolution Microscope (Nikon) using a 100× oil objective.

Calcium imaging of human iPSC derived CMs. Calcium imaging was performed as previously described (29). Briefly, CMs were dissociated and seeded on 35 mm glass-bottom dishes (Thermo Fisher Scientific). Calcium imaging was evaluated on 3 days after plating. CMs were loaded with the fluorescent calcium indicator Fluo-4-AM (Thermo Fisher Scientific, F14201) at 5 μM for 20 min in Tyrode's solution (Sigma-Aldrich, T2397). The calcium transients of spontaneous beating iPSC-CMs were measured at 37° C. using a Nikon A1R+ confocal microscope. Data were processed by Fiji software and analyzed using Microsoft Excel.

AAV delivery to differentiated iPSC-derived CMs. The cardiac troponin T (cTnT) promoter was extracted from the pAAV:cTNT::Luciferase (Addgene plasmid #69915) (30) from William Pu's lab (Harvard). The N-terminal and C-terminal regions of ABEmax-VRQR-SpCas9 were extracted from CMV_Npu-ABEmax N-terminal (Addgene plasmid #137173) (31) and hu6 HGPS sgRNA expression and ABE7.10max VRQR C-terminal AAV vectors (Addgene plasmid #154430) (21) from David Liu's lab, respectively. These inserts were subcloned into pSSV9 single-stranded AAV plasmid using In-Fusion cloning. AAV vectors were digested using SmaI and AhdI to confirm intact inverted terminal repeat (ITR) integrity. AAV viruses were generated with serotype 6 (AAV6) and serotype 9 (AAV9) capsids in the Boston Children's Hospital Viral Core. AAV6 viruses were infected into homozygous (R634Q/R634Q) iPSC-CMs at day 40 post-differentiation with 4×10⁵ vg/cell. Twenty days post infection, DNA was extracted.

Generating Rbm20^{R636Q} knock-in mice. Rbm20^{R636Q} knock-in mice were generated using CRISPR-Cas9 technology with ssODN template as described previously (32). The sgRNA for exon 9 of Rbm20 was cloned into pSpCas9(BB)-2A-GFP (px458). The sgRNAs were transcribed using the MEGA shortscript T7 Transcription kit and purified by MEGA clear kit (Life Technologies). Cas9 mRNA, Rbm20 sgRNA and ssODN containing the Rbm20^{R636Q} mutation were injected into mouse pronuclei and cytoplasm. Mouse embryos were transferred to a surrogate dam for gestation. F₀ generation pups were sequenced, and positive founders were bred to wild-type C57/BL6 mice. F₁ generation Rbm20^{R636Q} knock-in mice were identified by sequencing. Tail genomic DNA was extracted and used for genotyping.

Systemic AAV9 delivery in vivo. P5 homozygous (R636Q/R636Q) mice were injected intraperitoneally with 100 μl of AAV9 containing N-terminal- and C-terminal-ABEmax-VRQR-SpCas9-sgRNA (total of 2.5×10¹⁴ vg/kg) using an ultrafine BD insulin syringe (Becton Dickinson).

Transthoracic echocardiography. Cardiac function was assessed by two-dimensional transthoracic echocardiography using a VisualSonics Vevo2100 imaging system. Fractional shortening, left ventricular internal diameter at end diastole (LVIDd), and end systole (LVIDs) were measured using M-mode tracing. All measurements were performed by an operator blinded to this study.

Extraction of genomic DNA. Genomic DNA of iPSCs, iPSC-CMs and murine hearts was extracted using DirectPCR lysis reagent (VIAGEN) according to the manufacturer's protocol. Extracted genomic DNA was amplified using PrimeSTAR GXL DNA polymerase (TAKARA Bio) according to the manufacturer's protocol. PCR products were sequenced and analyzed by EditR for gene editing efficiency. PCR primers are listed in Table 1.

RNA isolation, RT-PCR, and qRT-PCR. Total RNA was extracted from iPSC-CMs at day 40 post-differentiation and murine hearts at 6-weeks post injection using miRNeasy (Qiagen), and cDNA was reverse-transcribed using iScript Reverse Transcription Supermix (Bio Rad Laboratories) according to the manufacturer's protocol. For RT-PCR, cDNA was amplified using PrimeSTAR GXL DNA polymerase (TAKARA Bio). For qRT-PCR, gene expression was measured using KAPA SYBR FAST Master mix (KAPA) and quantified by the Ct method. For normalization of qRT-PCR, we used 18s. RT-PCR and qRT-PCR primers are listed in Table 1.

RNA-seq analysis. Library prep from total RNA was performed using KAPA mRNA Hyper prep kit (Roche, KK8581) according to the manufacturer's protocol. Sequencing was performed on an Illumina Nextseq 500 system using the 75 bp high output sequencing kit for pair-end sequencing. Trim Galore (available on the world wide web at bioinformatics.babraham.ac.uk/projects/trim_galore/) was used for quality and adapter trimming. The qualities of RNA-sequencing libraries were estimated by mapping the reads onto human transcript and ribosomal RNA sequences (Ensembl release 89) using Bowtie (v2.3.4.3) (33). STAR (v2.7.2b) (34) was employed to align the reads onto the human genome (hg38). SAMtools (v1.9) (35) was employed to sort the alignments, and HTSeq Python package (36) was employed to count reads per gene. edgeR R Bioconductor package (37-39) was used to normalize read counts and identify differentially expressed (DE) genes. DE genes (fold change>2 in homozygous compared to normal, adjusted p-value <0.05) were analyzed in experiments using iPSC-CMs or murine hearts. Gene ontology (GO) analysis was performed using Metascape (40) and selective GO terms were shown. SpliceFisher (available at github.com/jiwoongbio/SpliceFisher) was used to identify differential alternative splicing events and to calculate PSI (percent spliced in) values.

TABLE 1
List of primers used in this study.

| Primer function | Primer name | Primer sequence |
|---|---|---|
| Generating iPSCs | R634Q-sgRNA-FW | CACCGCTCACCGGACTACGAGACCG (SEQ ID |
| (RBM20^{R634Q}) | | NO: 80) |
| | R634Q-sgRNA-RV | AAACCGGTCTCGTAGTCCGGTGAGC (SEQ ID |
| | | NO: 81) |
| | RBM20-Ex9-FW | GCCAGTGCTGTGCTTAGGA(SEQ ID NO: |
| | | 82) |
| | RBM20-Ex9-RV | TGGTGTTTGCGATCATGTGC (SEQ ID NO: |
| | | 83) |
| | ssODN-RBM20- | GTCTGTGTGTGGGTGGGGTGGGATGGGAGGTGTG |
| | R634Q | AAGATTCTAAATCCTGCTCCTTGGCTCCCTCACA |
| | | GATATGGCCCAGAAAGGCCGCAGTCTCGTAGTCC |
| | | GGTGAGCCGGTCACTCTCCCCGAGG (SEQ ID |
| | | NO: 84) |
| Base editing | R634Q-sgRNA1-FW | CACCGCCGCAGTCTCGTAGTCCGG (SEQ ID |
| sgRNAs | | NO: 85) |
| (RBM20^{R634Q}) | R634Q-sgRNA1-RV | AAACCCGGACTACGAGACTGCGGC (SEQ ID |
| | | NO: 86) |
| | R634Q-sgRNA2-FW | CACCGAGGCCGCAGTCTCGTAGTCC (SEQ ID |
| | | NO: 87) |
| | R634Q-sgRNA2-RV | AAACGGACTACGAGACTGCGGCCTC (SEQ ID |
| | | NO: 88) |
| | R634Q-sgRNA3-FW | CACCGAGGCCGCAGTCTCGTAGTCCG (SEQ ID |
| | | NO: 89) |
| | R634Q-sgRNA3-RV | AGACCGGACTACGAGACTGCGGCCTC (SEQ ID |
| | | NO: 90) |
| | R634Q-sgRNA4-FW | CACCGCGCAGTCTCGTAGTCCGGTG (SEQ ID |
| | | NO: 91) |
| | R634Q-sgRNA4-RV | AAACCACCGGACTACGAGACTGCGC (SEQ ID |
| | | NO: 92) |
| Human qRT-PCR | qRT-18SrRNA-FW | ACCGCAGCTAGGAATAATGGA (SEQ ID NO: |
| | | 93) |
| | qRT-18SrRNA-RV | GCCTCAGTTCCGAAAACCA (SEQ ID NO: |
| | | 94) |
| | qRT-TTN-N2B-FW | CCAATGAGTATGGCAGTGTCA(SEQ ID NO: |
| | | 95) |
| | qRT-TTN-N2B-RV | TACGTTCCGGAAGTAATTTGC (SEQ ID NO: |
| | | 96) |
| Off-target | OT1-FW | TTTGTTGAGGGCAGAGCCAA(SEQ ID NO: |
| | | 97) |
| | OT1-RV | TGGCACTCTTTGCTTGGTGA(SEQ ID NO: |
| | | 98) |
| | OT2-FW | GACTCTCTGTGGGCCTTCAAAGATGGA(SEQ |
| | | ID NO: 99) |
| | OT2-RV | TATTGGCGCTCGTCTGCCCAATCTC (SEQ ID |
| | | NO: 100) |
| | OT3-FW | CTTTCCTGACTACTTCCCTGGT (SEQ ID NO: |
| | | 101) |
| | OT3-RV | GAGTCTGGCAGTGGAACAAGA (SEQ ID NO: |
| | | 102) |
| | OT4-FW | GCAACTTGGCAAAGGGAAGAAAAACA (SEQ ID |
| | | NO: 103) |
| | OT4-RV | CAGAGTACCACTGCCTACCCACTACAA (SEQ |
| | | ID NO: 104) |
| | OT5-FW | GCATGTGTCTCCGGGTTCAA (SEQ ID NO: |
| | | 105) |
| | OT5-RV | GCGGCTTTCCCACTGAAATC (SEQ ID NO: |
| | | 106) |
| | OT6-FW | TGAACTGGACCCCCGAGGTGTAGCC (SEQ ID |
| | | NO: 107) |
| | OT6-RV | TTTCCTAAGAGTCGGTCGGCTTGAG (SEQ ID |
| | | NO: 108) |
| | OT7-FW | ATCCTTTGGGTCCAACCAGC (SEQ ID NO: |
| | | 109) |
| | OT7-RV | AGAGCACTTGGATAGTTGGCT (SEQ ID NO: |
| | | 110) |
| | OT8-FW | GCCCCCTTAGGTGTGCCCGTATCTT (SEQ ID |
| | | NO: 111) |
| | OT8-RV | GCAGTGAGGCTGTGTAGTCTTGGGC (SEQ ID |
| | | NO: 112) |
| Generating iPSCs | R636S-sgRNA-FW | CACCGACCGGCTCACCGGACTACG (SEQ ID |
| (RBM20^{R636S}) | | NO: 113) |
| | R636S-sgRNA-RV | AAACCGTAGTCCGGTGAGCCGGTC (SEQ ID |
| | | NO: 114) |
| | SSODN-RBM20- | GTGTGGGTGGGGTGGGATGGGAGGTGTGAAGATT |
| | R636S | CTAAATCCTGCTCCTTGGCTCCCTCACAGATATG |
| | | GCCCAGAAAGGCCGCGGTCTAGTAGTCCGGTGAG |
| | | CCGGTCACTCTCCCCGAGGTCCCAC (SEQ ID |
| | | NO: 115) |
| PegRNA (PBS | PegRNA-spacer-FW | CACCGATATGGCCCAGAAAGGCCGGTTTT (SEQ |
| 11 nt, RT 17 nt) | | ID NO: 116) |
| | PegRNA-spacer-RV | CTCTAAAACCGGCCTTTCTGGGCCATATC (SEQ |
| | | ID NO: 117) |
| | Scaffold-FW | AGAGCTAGAAATAGCAAGTTAAAATAAGGCTAGT |
| | | CCGTTATCAACTTGAAAAAGTGGCACCGAGTCG |
| | | (SEQ ID NO: 118) |
| | Scaffold-RV | GCACCGACTCGGTGCCACTTTTTCAAGTTGATAA |
| | | CGGACTAGCCTTATTTTAACTTGCTATTTCTAG |
| | | (SEQ ID NO: 119) |
| | PegRNA-FW | GTGCGGACTACGAGAGCGCGGCCTTTCTGGGC |
| | | (SEQ ID NO: 120) |
| | PegRNA-RV | AAAAGCCCAGAAAGGCCGCGCTCTCGTAGTCC |
| | | (SEQ ID NO: 121) |
| | PegRNA-2ndnick- | CACCGCTCACCGGACTACGAGAGCG (SEQ ID |
| | FW | NO: 122) |
| | PegRNA-2ndnick- | AAACCGCTCTCGTAGTCCGGTGAGC (SEQ ID |
| | RV | NO: 123) |
| EpegRNA (PBS | EpegRNA-FW | GTGCGGACTACGAGAGCGCGGCCTTTCTGGGCTT |
| 11 nt, RT 17 nt) | | GACGCGGTTCTATCTAGTTACGCGTTAAACCAAC |
| | | TAGAAA(SEQ ID NO: 124) |
| | EpegRNA-RV | AAAATTTCTAGTTGGTTTAACGCGTAACTAGATA |
| | | GAACCGCGTCAAGCCCAGAAAGGCCGCGCTCTCG |
| | | TAGTCC (SEQ ID NO: 125) |
| ABE-SpCas9- | AgeI-ABEmax-FW | TTTTTTTCAGGTTGGACCGGTGCCACCATGAAAC |
| variants-2A-GFP | | GGACAGCC (SEQ ID NO: 126) |
| | ABEmax-RV | GCCCACAGAGTTGGTGCCGAT (SEQ ID NO: |
| | | 127) |
| | SpCas9-FW | TCGGCACCAACTCTGTGGGC (SEQ ID NO: |
| | | 128) |
| | ApaI-SpCas9-RV | GCTGTTTCCCCTGGCCAGAGG (SEQ ID NO: |
| | | 129) |
| ABE8e-SaCas9- | Age1-ABE8e-FW | TTTTTTTCAGGTTGGACCGGTGCCACCATGAAAC |
| 2A-GFP | | GGACAGCCG (SEQ ID NO: 130) |
| | D10A-SaCas9-RV | TTGATGCTCTGGATGAAGCTCCGC (SEQ ID |
| | | NO: 131) |
| AAV-cTnT- | XhoI-PacI-cTnT-F1 | AGAAGAAATATAAGACTCGAGTTAATTAAGAGGT |
| ABEmax-VRQR- | | CGGGATAAAAGCAGTCTGGGC (SEQ ID NO: |
| SpCas9-N and -C | | 132) |
| | AgeI-cTnT-R1 | CCGTTTCATGGTGGCACCGGTTCCCACGGAGCGG |
| | | TGGGT (SEQ ID NO: 133) |
| | U6-gRNA-F1 | GGTGGGCTCTATGGGCGGCCGAGGGCCTATTTCC |
| | | CATGATTCC (SEQ ID NO: 134) |
| | Xbal-NotI-U6- | GCTGGCGCGCCTTTTTCTAGAGCGGCCGCAAAAA |
| | gRNA-R1 | AAGCACCGACTCGGTGC (SEQ ID NO: 135) |
| | XhoI-U6-gRNA-F1 | CGGTGGGCTCTATGGCTCGAGAGGGCCTATTTCC |
| | | CATGATTCC (SEQ ID NO: 136) |
| Generating a | Rbm20-sgRNA-FW | CACCGCTCATTGGACTTCGAGAACG (SEQ ID |
| mouse model | | NO: 137) |
| (Rbm20^{R636Q}) | Rbm20-sgRNA-RV | AAACCGTTCTCGAAGTCCAATGAGC (SEQ ID |
| | | NO: 138) |
| | Rbm20-Ex9-FW | GGTAGAGGGCAGAGAGTGTCTTAGGG (SEQ ID |
| | | NO: 139) |
| | Rbm20-Ex9-RV | CCTTCGAGTCGCTCATCCAACTCAGC (SEQ ID |
| | | NO: 140) |
| | ssODN-Rbm20- | GTCTCCATCTGGGTGATGCAGGTTACGAGCTCTG |
| | R636Q | CAGAGTCTAAACCCTGTCTCTTCCCTTCCTCCCA |
| | | GGTATGGTCCAGAGCGGCCACagTCTCGAAGTCC |
| | | AATGAGCCGATCACTCTCCCCAAGA(SEQ ID |
| | | NO: 141) |
| Base editing | R636Q-sgRNA-FW | CACCGCCACAGTCTCGAAGTCCAA (SEQ ID |
| sgRNA | | NO: 142) |
| (Rbm20^{R636Q}) | R636Q-sgRNA-RV | AAACTTGGACTTCGAGACTGTGGC (SEQ ID |
| | | NO: 143) |
| Mouse RT-PCR | Rbm20-FW | ATGGCTTACACAGAAGCCGC (SEQ ID NO: |
| | | 144) |
| | Rbm20-RV | CCTTCGAGTCGCTCATCCAA (SEQ ID NO: |
| | | 145) |
| Mouse qRT-PCR | qRT-mmu18SrRNA- | GTAACCCGTTGAACCCCATT (SEQ ID NO: |
| | FW | 146) |
| | qRT-mmu18SrRNA- | CCATCCAATCGGTAGTAGCG (SEQ ID NO: |
| | RV | 147) |
| | qRT-Ttn-N2B-FW | GGAGTACACCTGCAAAGCCT (SEQ ID NO: |
| | | 148) |
| | qRT-Ttn-N2B-RV | TGCGGCTTAGGTTCAGGAAG (SEQ ID NO: |
| | | 149) |
| | qRT-Ttn-N2BA-FW | GGAGTACACCTGCAAAGCCT (SEQ ID NO: |
| | | 148) |
| | qRT-Ttn-N2BA-RV | CCTTGGGCCTGGAGAGAAAG (SEQ ID NO: |
| | | 150) |

### Example 2—Results

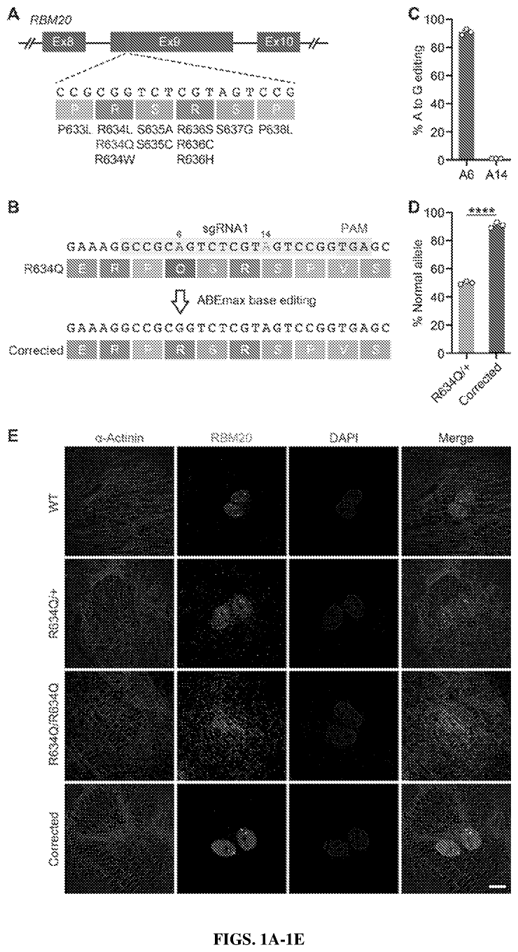

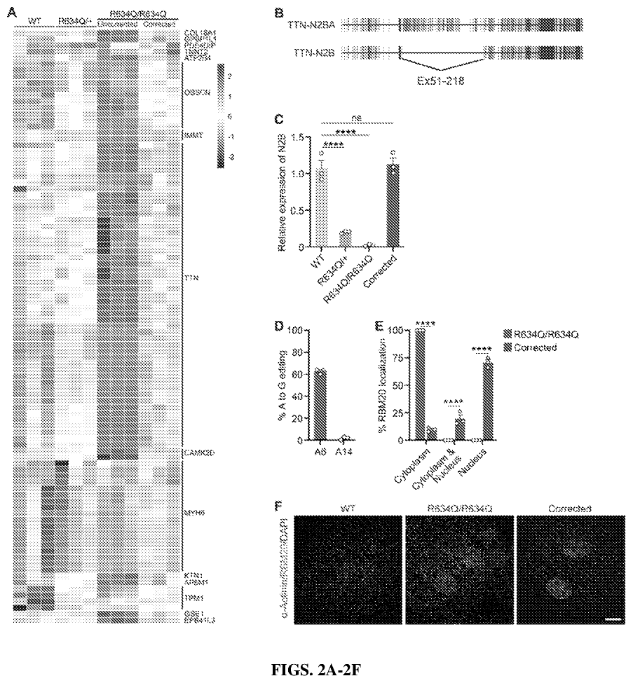

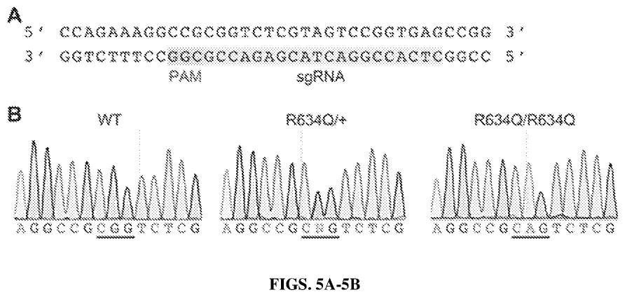

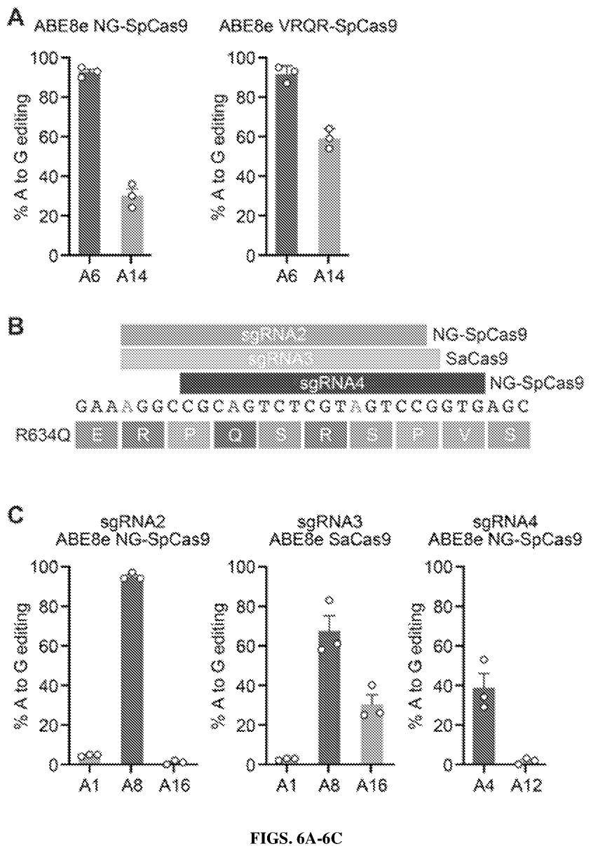

The RBM20^{R634Q} mutation (c.1901 G>A) in the RS-rich region is caused by transition of guanine to adenine, which is suitable for adenine base editing (ABE) (19, 20) (FIGS. 1A and 1B). To test whether ABE could be adapted to precisely correct this mutation, human isogenic induced pluripotent stem cell (iPSC) lines with heterozygous (R634Q/+) and homozygous (R634Q/R634Q) mutations were generated from healthy control iPSCs using CRISPR-Cas9 gene editing (FIGS. 5A and 5B). To evaluate the efficiency of ABE, four single guide RNAs (sgRNAs) (Table 2) were designed and each sgRNA (sgRNA 1-4) subcloned into the All-In-One ABE8e vectors that express different SpCas9 variants or SaCas9. Initially, each sgRNA was tested with different ABE8e variants but potentially detrimental bystander mutations were observed in addition to on target editing (FIGS. 6A-6C). Therefore, the base editor was switched to ABEmax, which has a narrower editing window. The combination of sgRNA1 and All-In-One ABEmax-VRQR-SpCas9, which has an on-target site at position A6 (FIG. 1B), showed high efficiency of A to G editing (89%) without inducing notable bystander mutations (<1%) (FIG. 1C). In addition, ABEmax-VRQR-SpCas9 editing of the R634Q/+ mutation in iPSCs increased the percentage of the normal RBM20 allele from 50% to 91% (FIG. 1D). Therefore, ABEmax-VRQR-SpCas9 (hereafter referred to as ABE) was selected for further studies.

TABLE 2
Single guide RNAs of base editing for the
RBM20^{R634Q} mutation.

| Base | | | |
|---|---|---|---|
| editing | | | |
| sgRNA | sgRNA sequence | PAM | Cas9 |
| sgRNA1 | GCCGCAGTCTCGTAGTCCGG | TGA | NG- or VRQR- |
| | (SEQ ID NO: 1) | | SpCas9 |
| sgRNA2 | AGGCCGCAGTCTCGTAGTCC | GG | NG-SpCas9 |
| | (SEQ ID NO: 2) | | |
| sgRNA3 | AGGCCGCAGTCTCGTAGTCCG | GTGAG | SaCas9 |
| | (SEQ ID NO: 3) | | |
| sgRNA4 | CGCAGTCTCGTAGTCCGGTG | AG | NG-SpCas9 |
| | (SEQ ID NO: 4) | | |

In healthy human CMs derived from iPSCs (iPSC-CMs), RBM20 was localized predominantly to the nucleus (FIG. 1E). In contrast, in R634Q/R634Q iPSC-CMs, RBM20 was localized to cytoplasmic RNP granules, while R634Q/+ iPSC-CMs showed RBM20 distributed in both the nucleus and cytoplasm (FIG. 1E). To correct the RBM20^{R634Q} mutation, ABE and sgRNA1 were used, and nuclear localization of RBM20, absence of RNP granule formation, and normal architecture of sarcomeric structures marked by α-actinin were observed (FIG. 1E).

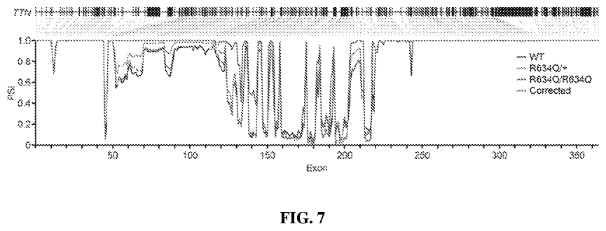

In addition to RNP granule formation, mutations of RBM20 also cause RNA splicing defects. To analyze the alternative splicing of genes regulated by RBM20, RNA-seq analysis was performed on normal, uncorrected and ABE-corrected R634Q/R634Q iPSC-CMs. As shown in the heatmap (FIG. 2A), 15 genes showed aberrant splicing patterns in uncorrected CMs, including genes encoding cardiac sarcomere and calcium signaling proteins, such as TTN, myosin heavy chain 6 (MYH6), Troponin T2 (TNNT2), and calcium/calmodulin-dependent protein kinase II6 (CAMK2D). Since TTN mis-splicing represents a major indicator of RBM20-associated DCM, the exon-inclusion ratio was measured by percent spliced in (PSI) to assess the splicing pattern of the TTN gene (FIG. 7). Normal splicing of TTN produces a rigid isoform, termed N2B, which lacks exon 51 through exon 218 (FIG. 2B). In uncorrected R634Q/R634Q iPSC-CMs, exons 51 to 218 were not spliced properly, generating the N2BA isoform (FIG. 7). This mis-spliced isoform reduces cardiac stiffness, leading to DCM. Similarly, R634Q/+ iPSC-CMs also showed an abnormal alternative splicing pattern compared to normal iPSC-CMs (FIG. 7). Conversely, ABE-corrected iPSC-CMs displayed normal TTN splicing pattern as healthy iPSC-CMs (FIG. 7). The expression of the N2B isoform in ABE-corrected iPSC-CMs was also validated by qRT-PCR analysis (FIG. 2C). Therefore, ABE correction of the RBM20^{R634Q} mutation was effective in restoring proper RNA splicing.

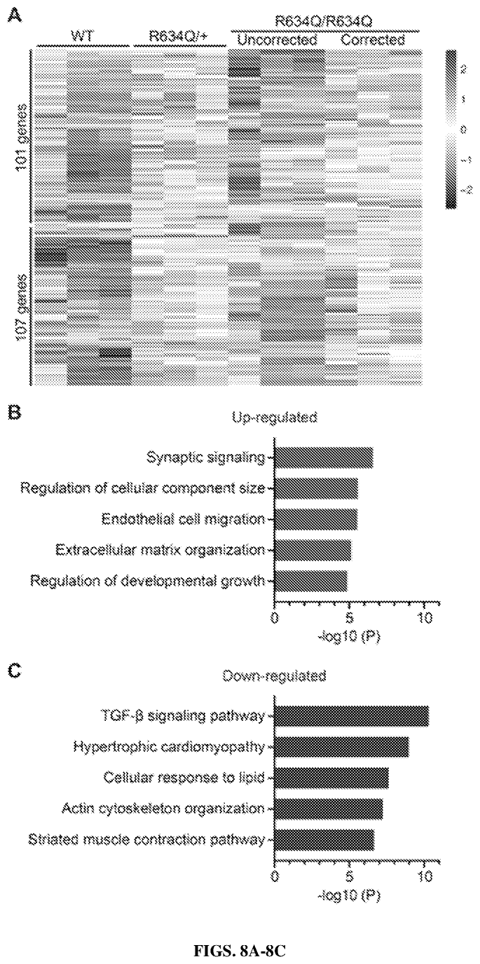

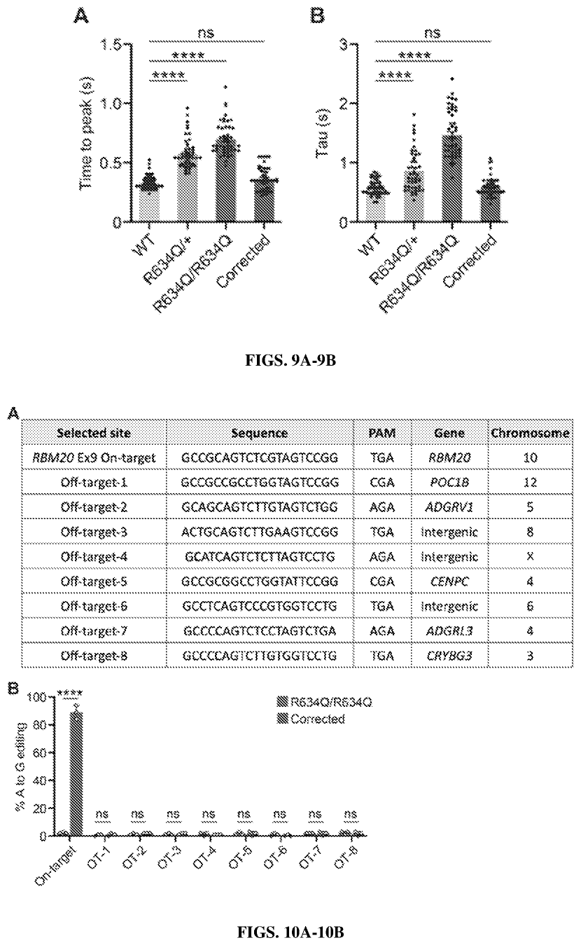

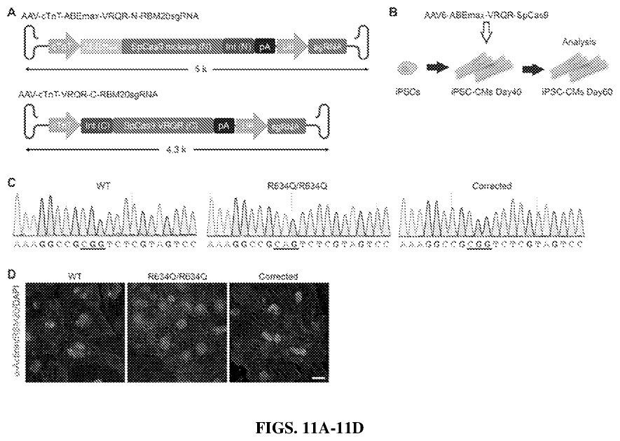

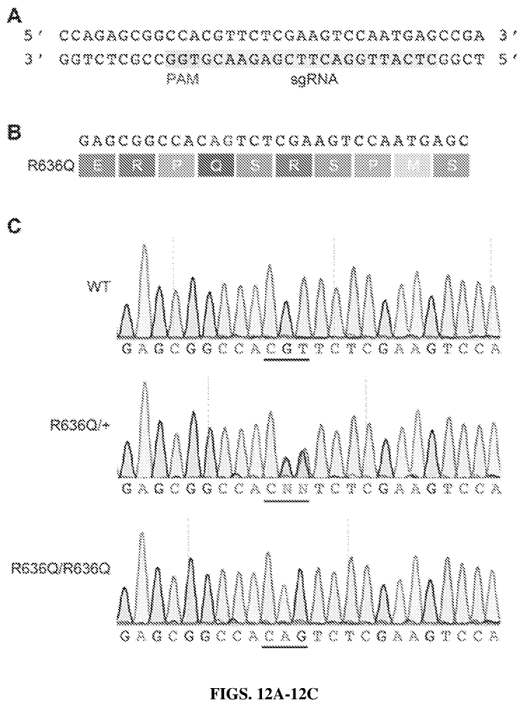

Dysregulation of gene expression and calcium handling are common pathogenic phenotypes seen in CMs with RBM20 mutations. To assess the transcriptional consequences of the RBM20^{R634Q} mutation and the effect of ABE gene editing, RNA-seq and gene Ontology (GO) analyses were performed on normal, uncorrected and ABE-corrected R634Q/R634Q iPSC-CMs (FIGS. 8A-8C). Down-regulated genes in R634Q/R634Q iPSC-CMs included categories related to cardiomyopathy and striated muscle contraction, consistent with the phenotype of DCM. The abnormal transcriptome seen in R634Q/R634Q iPSC-CMs was recovered following ABE editing of iPSC-CMs. Calcium transient kinetics, including time to peak and decay rate (tau), were abnormally elevated in both R634Q/+ and R634Q/R634Q iPSC-CMs (FIGS. 9A and 9B). After ABE-mediated correction, R634Q/R634Q iPSC-CMs displayed normal calcium transient kinetics, like those of healthy control iPSC-CMs (FIGS. 9A and 9B), indicating restoration of calcium release and reuptake. To assess possible off-target editing, genomic DNA of the top eight predicted off-target sites was sequenced and no detectable genomic alterations were found at any of the potential off-target sites (FIGS. 10A and 10B).

Restoration of RBM20 nuclear localization and elimination of RNP granules are required for proper cardiac function. However, it was unclear whether eradicating accumulated RNP granules from differentiated CMs would be effective. To confirm the elimination of RNP granules in differentiated iPSC-CMs, adeno-associated virus serotype-6 (AAV6) and the trans-splicing intein system (21) were used to deliver ABE components driven by the cardiac troponin T promoter (cTnT), into differentiated R634Q/R634Q CMs (FIGS. 11A and 11B). After ABE correction, the corrected iPSC-CMs showed 63% editing efficiency at the genomic level (FIGS. 2D and 11C). Immunocytochemistry showed localization of RBM20 in the nucleus and elimination of RNP granules in the cytoplasm (FIGS. 2E, 2F, and 11D). These findings indicate that ABE rescues the normal cardiac phenotype by reversing the formation of RNP granules.

To evaluate the potential of ABE for in vivo DCM therapy, a Rbm20^{R636Q} mutant mouse model, which is analogous to the human RBM20^{R634Q} mutation, was generated (FIGS. 12A-12C). Cardiac function was measured in 4-week-old mutant mice by echocardiography. Homozygous (R636Q/R636Q) mice showed dramatically reduced fractional shortening (% FS) (FIGS. 3A and 3B). Left ventricular internal dimensions during end-diastole (LVIDd) and end-systole (LVIDs) were substantially increased in R636Q/R636Q mice compared to wild type (WT) mice. Heterozygous (R636Q/+) mice demonstrated mild cardiac dysfunction (FIGS. 3A and 3B). Hearts of R636Q/R636Q mice at 12 weeks of age showed morphological characteristics consistent with DCM, including atrial and ventricular dilation (FIG. 3C). Thus, the R636Q/R636Q mice recapitulate the phenotype of DCM patients harboring the RBM20^{R634Q} mutation.

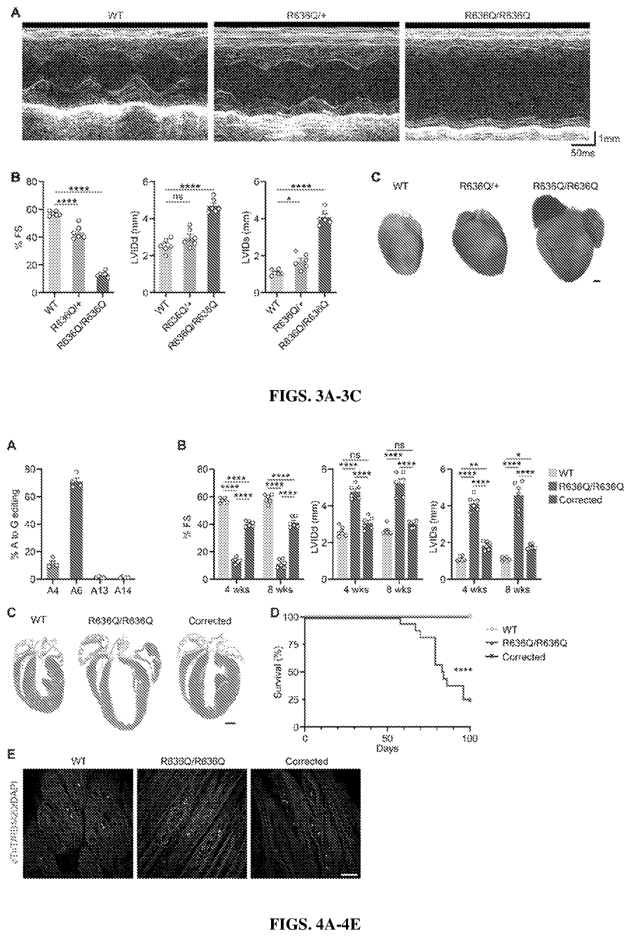

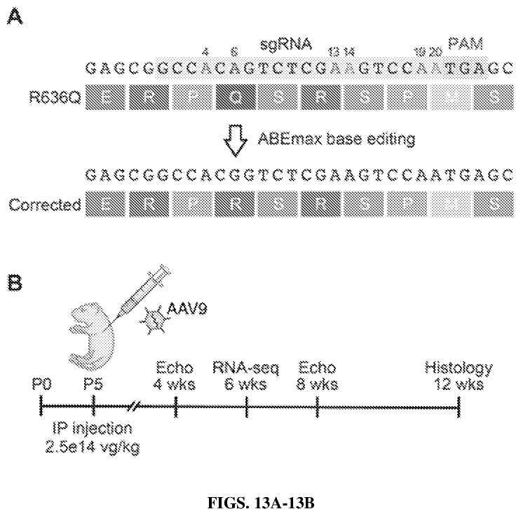

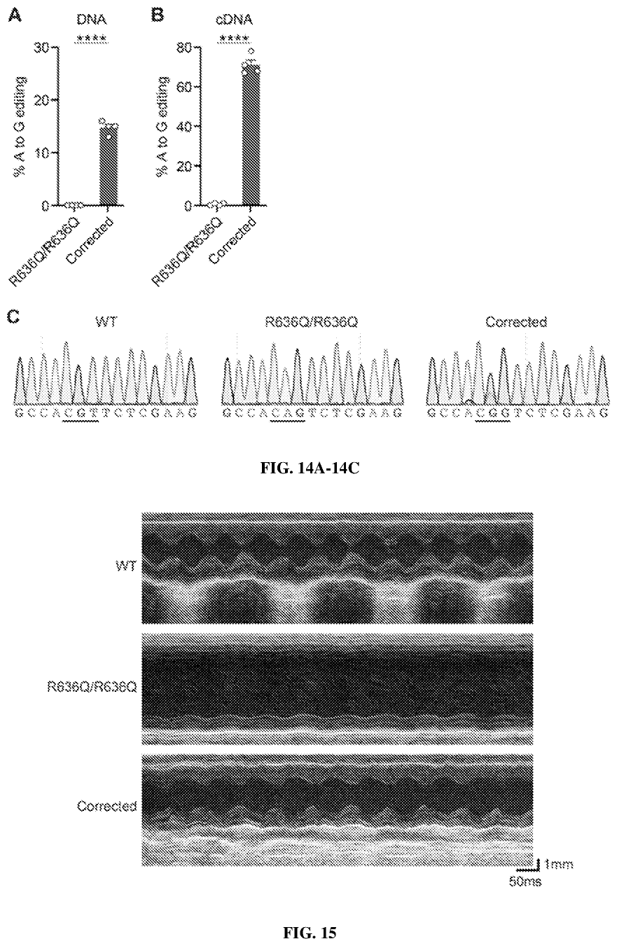

The R636Q/R636Q mutant mouse model enabled the assessment of in vivo correction of the RBM20^{R634Q} mutation by ABE. For ABE correction in mice, the targeted adenine is positioned in the sgRNA at the A6 position, with possible bystander mutations found at A14 and A20, and silent mutations at A4, A13 and A19 (FIG. 13A). ABE components were administered by intraperitoneal injection of AAV9 at a dose of 1.25×10¹⁴ vg/kg for each AAV (total 2.5×10¹⁴ vg/kg) to postnatal day 5 mice (FIG. 13B). To confirm whether ABE-mediated correction was effective in vivo, DNA editing efficiency was assessed in the heart. Six weeks post administration of ABE components by AAV, 15% of the targeted adenine underwent DNA editing (FIG. 14A). Genomic INDEL analysis underestimates ABE editing efficiency because other cell types, such as endothelial cells and cardiac fibroblasts, which comprise up to two-thirds of the cells of the heart, are not edited by the ABE components, which were expressed under the control of the cardiac-specific TnT promoter. Therefore, ABE gene editing was further evaluated at the cDNA level and 71% of RBM20 cDNA transcripts were found to have been precisely corrected (FIGS. 4A, 14B, and 14C).

To assess cardiac function after ABE correction, echocardiography was performed in R636Q/R636Q mice at 4- and 8-weeks after systemic delivery of ABE gene editing components. Uncorrected R636Q/R636Q mice exhibited severe heart failure and premature death at 2 to 3-months of age. In contrast, R636Q/R636Q mice receiving systemic ABE components showed a substantial improvement of LV function, as measured by fractional shortening (FIGS. 4B and 15). In addition, cardiac chamber size was also partially rescued (FIG. 4B). Histological analysis of ABE-corrected R636Q/R636Q hearts at 12-weeks post-AAV9 administration showed recovery from cardiac dilation, while untreated hearts revealed atrial and ventricular enlargement (FIG. 4C). Histological assessment revealed no significant fibrosis of the left ventricular myocardium in treated R636Q/R636Q mice (FIGS. 16A and 16B). Importantly, systemic delivery of ABE editing components also significantly increased the life span of the corrected R636Q/R636Q mice (FIG. 4D).

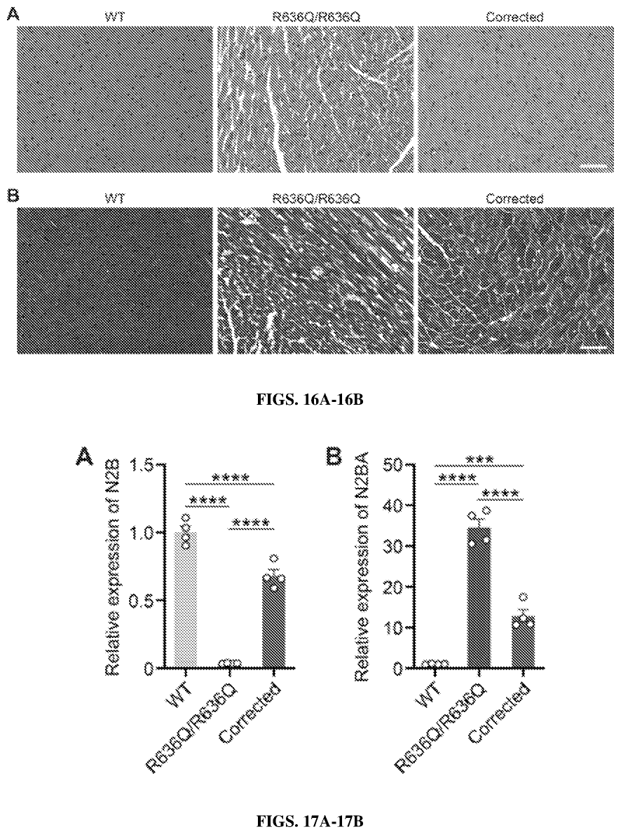

To determine whether the restoration of cardiac function was attributable to restored function of RBM20, the localization of RBM20 was evaluated in vivo. In WT mice, RBM20 was localized to the nucleus without formation of RNP granules (FIG. 4E). In contrast, CMs of R636Q/R636Q mice displayed RNP granules in the perinuclear region, similar to R634Q/R634Q iPSC-CMs. ABE-mediated correction of R636Q/R636Q mice restored RBM20 localization to the nucleus and eliminated RNP granules as assessed by immunohistochemistry (FIG. 4E). Next, the splicing pattern of Ttn was evaluated in R636Q/R636Q mice following systemic ABE gene editing. qRT-PCR analysis showed a reduction of the rigid N2B isoform and an increase of the compliant N2BA isoform in the R636Q/R636Q mice (FIGS. 17A and 17B). Conversely, ABE corrected R636Q/R636Q hearts showed partial restoration (68%) of the N2B isoform and a reduction (63%) of the N2BA isoform (FIGS. 17A and 17B).

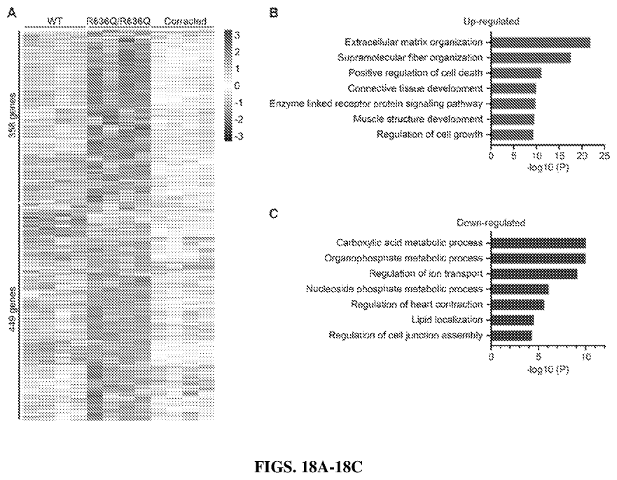

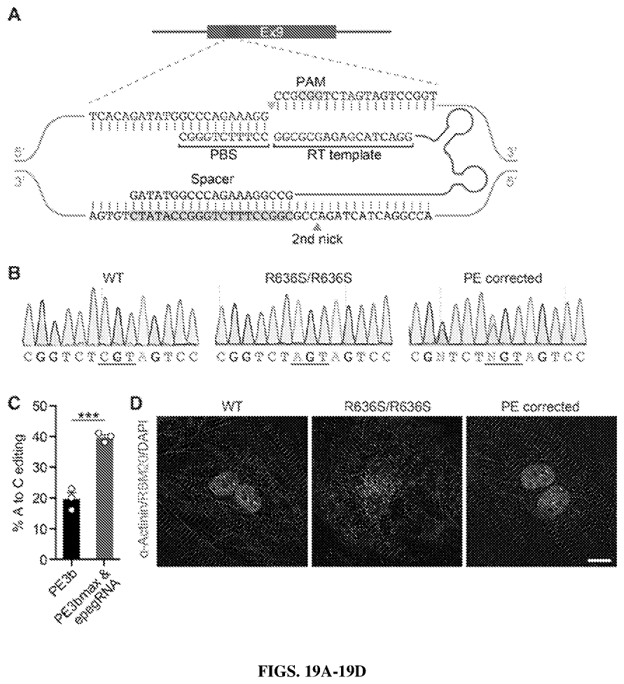

To determine whether ABE gene editing normalized gene expression in R636Q/R636Q mice, RNA-seq analysis was performed. ABE gene editing rescued the transcriptional profile of corrected R636Q/R636Q mice, while R636Q/R636Q mice showed altered gene expression and GO terms including heart contraction and extracellular matrix (FIGS. 18A-18C). These findings demonstrate that ABE gene editing protects the heart without inducing adverse events, such as inflammation and cell damage.

Despite the high efficiency of ABE gene editing of the RBM20^{R634Q} mutation, BE cannot correct all RBM20 mutations due to the limited editing window, unwanted bystander editing, and the lack of a proper PAM sequence near some target nucleotides. To overcome these limitations, a prime editing (PE) strategy was developed for the p.R636S (c.1906 C>A) mutation. The PE system includes a Cas9 nickase fused to reverse transcriptase and a prime-editing guide RNA (pegRNA) that contains a primer binding site (PBS) and a reverse transcription (RT) template to enable the incorporation of the intended sequence at the target site in the genome (22). Recent studies reported that engineered pegRNAs (epegRNAs), which have an additional RNA motif to protect the 3′ extension from degradation, coupled with a new variant of the prime editor (PEmax) improved PE efficiency (23, 24). A pegRNA was designed with a PBS length of 11nt and RT template length of 17nt (FIG. 19A). To optimize the editing efficiency, the nicking sgRNA was selected for the PE3b system and the epegRNA created by inserting a structured RNA motif. To evaluate the efficiency of PE, an isogenic iPSC line with the homozygous RBM20^{R636S} (R636S/R636S) mutation was generated. The PE3bmax coupled with epegRNA resulted in A to C editing with an efficiency of 40% without unintended genomic edits (FIGS. 19B and 19C). Whereas RNP granules were detected in the cytoplasm of uncorrected R636S/R636S iPSC-CMs, a normal pattern of nuclear localization was observed in PE-corrected iPSC-CMs (FIG. 19D).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. E. M. McNally, L. Mestroni, Dilated Cardiomyopathy: Genetic Determinants and Mechanisms. Circ Res 121, 731-748 (2017).
2. E. Jordan, L. Peterson, T. Ai, B. Asatryan, L. Bronicki, E. Brown, R. Celeghin, M. Edwards, J. Fan, J. Ingles, C. A. James, O. Jarinova, R. Johnson, D. P. Judge, N. Lahrouchi, R. H. Lekanne Deprez, R. T. Lumbers, F. Mazzarotto, A. Medeiros Domingo, R. L. Miller, A. Morales, B. Murray, S. Peters, K. Pilichou, A. Protonotarios, C. Semsarian, P. Shah, P. Syrris, C. Thaxton, J. P. van Tintelen, R. Walsh, J. Wang, J. Ware, R. E. Hershberger, Evidence-Based Assessment of Genes in Dilated Cardiomyopathy. Circulation 144, 7-19 (2021).
3. A. N. Rosenbaum, K. E. Agre, N. L. Pereira, Genetics of dilated cardiomyopathy: practical implications for heart failure management. Nat Rev Cardiol 17, 286-297 (2020).
4. K. M. Brauch, M. L. Karst, K. J. Herron, M. de Andrade, P. A. Pellikka, R. J. Rodeheffer, V. V. Michels, T. M. Olson, Mutations in ribonucleic acid binding protein gene cause familial dilated cardiomyopathy. J Am Coll Cardiol 54, 930-941 (2009).
5. H. C. Zahr, D. E. Jaalouk, Exploring the Crosstalk Between LMNA and Splicing Machinery Gene Mutations in Dilated Cardiomyopathy. Front Genet 9, 231 (2018).
6. T. M. Hey, T. B. Rasmussen, T. Madsen, M. M. Aagaard, M. Harbo, H. Molgaard, J. E. Moller, H. Eiskjaer, J. Mogensen, Pathogenic RBM20-Variants Are Associated With a Severe Disease Expression in Male Patients With Dilated Cardiomyopathy. Circ Heart Fail 12, e005700 (2019).
7. D. Lennermann, J. Backs, M. M. G. van den Hoogenhof, New Insights in RBM20 Cardiomyopathy. Curr Heart Fail Rep 17, 234-246 (2020).
8. D. Li, A. Morales, J. Gonzalez-Quintana, N. Norton, J. D. Siegfried, M. Hofmeyer, R. E. Hershberger, Identification of novel mutations in RBM20 in patients with dilated cardiomyopathy. Clin Transl Sci 3, 90-97 (2010).
9. A. M. Gacita, E. M. McNally, Genetic Spectrum of Arrhythmogenic Cardiomyopathy. Circ Heart Fail 12, e005850 (2019).
10. V. N. Parikh, C. Caleshu, C. Reuter, L. C. Lazzeroni, J. Ingles, J. Garcia, K. McCaleb, T. Adesiyun, F. Sedaghat-Hamedani, S. Kumar, S. Graw, M. Gigli, D. Stolfo, M. Dal Ferro, A. Y. Ing, R. Nussbaum, B. Funke, M. T. Wheeler, R. E. Hershberger, S. Cook, L. M. Steinmetz, N. K. Lakdawala, M. R. G. Taylor, L. Mestroni, M. Merlo, G. Sinagra, C. Semsarian, B. Meder, D. P. Judge, E. Ashley, Regional Variation in RBM20 Causes a Highly Penetrant Arrhythmogenic Cardiomyopathy. Circ Heart Fail 12, e005371 (2019).
11. T. Watanabe, A. Kimura, H. Kuroyanagi, Alternative Splicing Regulator RBM20 and Cardiomyopathy. Front Mol Biosci 5, 105 (2018).
12. W. Guo, S. Schafer, M. L. Greaser, M. H. Radke, M. Liss, T. Govindarajan, H. Maatz, H. Schulz, S. Li, A. M. Parrish, V. Dauksaite, P. Vakeel, S. Klaassen, B. Gerull, L. Thierfelder, V. Regitz-Zagrosek, T. A. Hacker, K. W. Saupe, G. W. Dec, P. T. Ellinor, C. A. MacRae, B. Spallek, R. Fischer, A. Perrot, C. Ozcelik, K. Saar, N. Hubner, M. Gotthardt, RBM20, a gene for hereditary cardiomyopathy, regulates titin splicing. Nat Med 18, 766-773 (2012).
13. J. W. Schneider, S. Oommen, M. Y. Qureshi, S. C. Goetsch, D. R. Pease, R. S. Sundsbak, W. Guo, M. Sun, H. Sun, H. Kuroyanagi, D. A. Webster, A. W. Coutts, K. A. Holst, B. S. Edwards, N. Newville, M. A. Hathcock, T. Melkamu, F. Briganti, W. Wei, M. G. Romanelli, S. C. Fahrenkrug, D. E. Frantz, T. M. Olson, L. M. Steinmetz, D. F. Carlson, T. J. Nelson, P. Wanek, Dysregulated ribonucleoprotein granules promote cardiomyopathy in RBM20 gene-edited pigs. Nat Med 26, 1788-1800 (2020).
14. K. Ihara, T. Sasano, Y. Hiraoka, M. Togo-Ohno, Y. Soejima, M. Sawabe, M. Tsuchiya, H. Ogawa, T. Furukawa, H. Kuroyanagi, A missense mutation in the RSRSP stretch of Rbm20 causes dilated cardiomyopathy and atrial fibrillation in mice. Sci Rep 10, 17894 (2020).
15. A. M. Fenix, Y. Miyaoka, A. Bertero, S. M. Blue, M. J. Spindler, K. K. B. Tan, J. A. Perez-Bermejo, A. H. Chan, S. J. Mayerl, T. D. Nguyen, C. R. Russell, P. P. Lizarraga, A. Truong, P. L. So, A. Kulkarni, K. Chetal, S. Sathe, N. J. Sniadecki, G. W. Yeo, C. E. Murry, B. R. Conklin, N. Salomonis, Gain-of-function cardiomyopathic mutations in RBM20 rewire splicing regulation and re-distribute ribonucleoprotein granules within processing bodies. Nat Commun 12, 6324 (2021).
16. C. Wang, Y. Zhang, M. Methawasin, C. U. Braz, J. Gao-Hu, B. Yang, J. Strom, J. Gohlke, T. Hacker, H. Khatib, H. Granzier, W. Guo, RBM20(S639G) mutation is a high genetic risk factor for premature death through RNA-protein condensates. J Mol Cell Cardiol 165, 115-129 (2022).
17. A. V. Anzalone, L. W. Koblan, D. R. Liu, Genome editing with CRISPR-Cas nucleases, base editors, transposases and prime editors. Nat Biotechnol 38, 824-844 (2020).
18. T. Nishiyama, R. Bassel-Duby, E. N. Olson, Toward CRISPR Therapies for Cardiomyopathies. Circulation 144, 1525-1527 (2021).
19. N. M. Gaudelli, A. C. Komor, H. A. Rees, M. S. Packer, A. H. Badran, D. I. Bryson, D. R. Liu, Programmable base editing of A*T to G*C in genomic DNA without DNA cleavage. Nature 551, 464-471 (2017).
20. M. F. Richter, K. T. Zhao, E. Eton, A. Lapinaite, G. A. Newby, B. W. Thuronyi, C. Wilson, L. W. Koblan, J. Zeng, D. E. Bauer, J. A. Doudna, D. R. Liu, Phage-assisted evolution of an adenine base editor with improved Cas domain compatibility and activity. Nat Biotechnol 38, 883-891 (2020).
21. L. W. Koblan, M. R. Erdos, C. Wilson, W. A. Cabral, J. M. Levy, Z. M. Xiong, U. L. Tavarez, L. M. Davison, Y. G. Gete, X. Mao, G. A. Newby, S. P. Doherty, N. Narisu, Q. Sheng, C. Krilow, C. Y. Lin, L. B. Gordon, K. Cao, F. S. Collins, J. D. Brown, D. R. Liu, In vivo base editing rescues Hutchinson-Gilford progeria syndrome in mice. Nature 589, 608-614 (2021).
22. A. V. Anzalone, P. B. Randolph, J. R. Davis, A. A. Sousa, L. W. Koblan, J. M. Levy, P. J. Chen, C. Wilson, G. A. Newby, A. Raguram, D. R. Liu, Search-and-replace genome editing without double-strand breaks or donor DNA. Nature 576, 149-157 (2019).
23. J. W. Nelson, P. B. Randolph, S. P. Shen, K. A. Everette, P. J. Chen, A. V. Anzalone, M. An, G. A. Newby, J. C. Chen, A. Hsu, D. R. Liu, Engineered pegRNAs improve prime editing efficiency. Nat Biotechnol, (2021).
24. P. J. Chen, J. A. Hussmann, J. Yan, F. Knipping, P. Ravisankar, P. F. Chen, C. Chen, J. W. Nelson, G. A. Newby, M. Sahin, M. J. Osborn, J. S. Weissman, B. Adamson, D. R. Liu, Enhanced prime editing systems by manipulating cellular determinants of editing outcomes. Cell 184, 5635-5652 e5629 (2021).
25. F. A. Ran, P. D. Hsu, J. Wright, V. Agarwala, D. A. Scott, F. Zhang, Genome engineering using the CRISPR-Cas9 system. Nat Protoc 8, 2281-2308 (2013).
26. T. P. Huang, K. T. Zhao, S. M. Miller, N. M. Gaudelli, B. L. Oakes, C. Fellmann, D. F. Savage, D. R. Liu, Circularly permuted and PAM-modified Cas9 variants broaden the targeting scope of base editors. Nat Biotechnol 37, 626-631 (2019).
27. Y. Zhang, C. Long, H. Li, J. R. McAnally, K. K. Baskin, J. M. Shelton, R. Bassel-Duby, E. N. Olson, CRISPR-Cpf1 correction of muscular dystrophy mutations in human cardiomyocytes and mice. Sci Adv 3, e1602814 (2017).
28. Y. L. Min, H. Li, C. Rodriguez-Caycedo, A. A. Mireault, J. Huang, J. M. Shelton, J. R. McAnally, L. Amoasii, P. P. A. Mammen, R. Bassel-Duby, E. N. Olson, CRISPR-Cas9 corrects Duchenne muscular dystrophy exon 44 deletion mutations in mice and human cells. Sci Adv 5, eaav4324 (2019).
29. F. Chemello, A. C. Chai, H. Li, C. Rodriguez-Caycedo, E. Sanchez-Ortiz, A. Atmanli, A. A. Mireault, N. Liu, R. Bassel-Duby, E. N. Olson, Precise correction of Duchenne muscular dystrophy exon deletion mutations by base and prime editing. Sci Adv 7, (2021).
30. Z. Lin, A. von Gise, P. Zhou, F. Gu, Q. Ma, J. Jiang, A. L. Yau, J. N. Buck, K. A. Gouin, P. R. van Gorp, B. Zhou, J. Chen, J. G. Seidman, D. Z. Wang, W. T. Pu, Cardiac-specific YAP activation improves cardiac function and survival in an experimental murine MI model. Circ Res 115, 354-363 (2014).
31. J. M. Levy, W. H. Yeh, N. Pendse, J. R. Davis, E. Hennessey, R. Butcher, L. W. Koblan, J. Comander, Q. Liu, D. R. Liu, Cytosine and adenine base editing of the brain, liver, retina, heart and skeletal muscle of mice via adeno-associated viruses. Nat Biomed Eng 4, 97-110 (2020).
32. B. R. Nelson, C. A. Makarewich, D. M. Anderson, B. R. Winders, C. D. Troupes, F. Wu, A. L. Reese, J. R. McAnally, X. Chen, E. T. Kavalali, S. C. Cannon, S. R. Houser, R. Bassel-Duby, E. N. Olson, A peptide encoded by a transcript annotated as long noncoding RNA enhances SERCA activity in muscle. Science 351, 271-275 (2016).
33. B. Langmead, S. L. Salzberg, Fast gapped-read alignment with Bowtie 2. Nat Methods 9, 357-359 (2012).
34. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics 29, 15-21 (2013).
35. H. Li, B. Handsaker, A. Wysoker, T. Fennell, J. Ruan, N. Homer, G. Marth, G. Abecasis, R. Durbin, S. Genome Project Data Processing, The Sequence Alignment/Map format and SAMtools. Bioinformatics 25, 2078-2079 (2009).
36. S. Anders, P. T. Pyl, W. Huber, HTSeq—a Python framework to work with high-throughput sequencing data. Bioinformatics 31, 166-169 (2015).
37. R. C. Gentleman, V. J. Carey, D. M. Bates, B. Bolstad, M. Dettling, S. Dudoit, B. Ellis, L. Gautier, Y. Ge, J. Gentry, K. Homik, T. Hothom, W. Huber, S. Iacus, R. Irizarry, F. Leisch, C. Li, M. Maechler, A. J. Rossini, G. Sawitzki, C. Smith, G. Smyth, L. Tierney, J. Y. Yang, J. Zhang, Bioconductor: open software development for computational biology and bioinformatics. Genome Biol 5, R80 (2004).
38. M. D. Robinson, D. J. McCarthy, G. K. Smyth, edgeR: a Bioconductor package for differential expression analysis of digital gene expression data. Bioinformatics 26, 139-140 (2010).
39. D. J. McCarthy, Y. Chen, G. K. Smyth, Differential expression analysis of multifactor RNA-Seq experiments with respect to biological variation. Nucleic Acids Res 40, 4288-4297 (2012).
40. Y. Zhou, B. Zhou, L. Pache, M. Chang, A. H. Khodabakhshi, O. Tanaseichuk, C. Benner, S. K. Chanda, Metascape provides a biologist-oriented resource for the analysis of systems-level datasets. Nat Commun 10, 1523 (2019).

What is claimed is:

1. A guide RNA (gRNA) comprising a targeting nucleic acid sequence selected from any one of SEQ ID NOs: 1-4.

2. The gRNA of claim 1, wherein the gRNA is a single-molecule guide RNA (sgRNA).

3. The gRNA of claim 1 or 2, wherein the gRNA is for modifying a sequence in the human RBM20 gene.

4. A composition comprising a gRNA that targets a mutation in human RBM20 and a base editor.

5. The composition of claim 4, wherein the base editor is an adenine base editor (ABE).

6. The composition of claim 4, wherein the gRNA is the gRNA of any one of claims 1-3.

7. The composition of claim 6, wherein the base editor is an adenine base editor (ABE).

8. The composition of any one of claims 4-7, wherein the base editor comprises a CRISPR/Cas nucleases linked to an adenosine deaminase.

9. The composition of claim 8, wherein the CRISPR/Cas nuclease is catalytically impaired.

10. The composition of claim 8 or 9, wherein the CRISPR/Cas nuclease is a Cas9 nuclease.

11. The composition of claim 10, wherein the Cas9 nuclease is isolated or derived from Streptococcus pyogenes (spCas9).

12. A nucleic acid comprising:
a sequence encoding a first gRNA of any one of claims 1-3,
a sequence encoding a base editor,
a sequence encoding a first promoter, wherein the first promoter drives expression of the sequence encoding the first gRNA, and
a sequence encoding a second promoter, wherein the second promoter drives expression of the sequence encoding the base editor.

13. The nucleic acid of claim 12, wherein the base editor is an adenine base editor (ABE).

14. The nucleic acid of claim 12 or 13, wherein the base editor comprises a CRISPR/Cas nuclease linked to an adenosine deaminase.

15. The nucleic acid of claim 14, wherein the CRISPR/Cas nuclease is catalytically impaired.

16. The nucleic acid of claim 14 or 15, wherein the CRISPR/Cas nuclease is a Cas9 nuclease.

17. The nucleic acid of claim 16, wherein the Cas9 nuclease is isolated or derived from Streptococcus pyogenes (spCas9), Staphylococcus aureus (SaCas9), Staphylococcus auricularis (SauCas9), or Staphylococcus lugdunensis (SlugCas9).

18. The nucleic acid any one of claims 12-17, wherein at least one of the sequence encoding the first promoter and the sequence encoding the second promoter comprises a cell-type specific promoter.

19. The nucleic acid of claim 18, wherein the cell-type specific promoter is a cardiomyocyte-specific promoter.

20. The nucleic acid of claim 19, wherein the muscle-specific promoter is a cardiac troponin T (cTnT) promoter.

21. The nucleic acid of any one of claims 12-20, wherein the sequence encoding the first promoter comprises a sequence encoding a U6 promoter, an H1 promoter, or a 7SK promoter.

22. The nucleic acid of any one of claims 12-21, wherein the nucleic acid comprises a DNA sequence.

23. The nucleic acid of any one of claims 12-22, wherein the nucleic acid comprises an RNA sequence.

24. The nucleic acid of any one of claims 12-23, wherein the nucleic acid further comprises a polyadenosine (polyA) sequence.

25. The nucleic acid of claim 24, wherein the polyA sequence is a mini polyA sequence.

26. A cell comprising the nucleic acid of any one of claims 12-25.

27. A composition comprising the nucleic acid of any one of claims 12-25.

28. A cell comprising the composition of claim 27.

29. A composition comprising the cell of claim 28.

30. A vector comprising the nucleic acid of any one of claims 12-25.

31. The vector of claim 30, wherein the vector further comprises a sequence encoding an inverted terminal repeat (ITR) of a transposable element.

32. The vector of claim 31, wherein the transposable element is a transposon.

33. The vector of claim 32, wherein the transposon is a Tn7 transposon.

34. The vector of claim 33, wherein the vector further comprises a sequence encoding a 5′ ITR of a T7 transposon and a sequence encoding a 3′ ITR of a T7 transposon.

35. The vector of any one of claims 30-34, wherein the vector is a non-viral vector.

36. The vector of claim 35, wherein the non-viral vector is a plasmid.

37. The vector of any one of claims 30-34, wherein the vector is a viral vector.

38. The vector of claim 37, wherein the viral vector is an adeno-associated viral (AAV) vector or an adenoviral vector.

39. The vector of claim 38, wherein the AAV vector is replication-defective or conditionally replication defective.

40. The vector of claim 38 or 39, wherein the AAV vector is a recombinant AAV vector.

41. The vector of any one of claims 38-40, wherein the AAV vector comprises a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11) or any combination thereof.

42. The vector of any one of claims 38-41, wherein the AAV vector comprises a sequence isolated or derived from an AAV vector of serotype 9 (AAV9).

43. The vector of any one of claims 38-42, wherein the AAV vector comprises a sequence isolated or derived from an AAV vector of serotype 2 (AAV2).

44. The vector of any one of claims 38-43, wherein the AAV vector comprises a sequence isolated or derived from an AAV2 and a sequence isolated or derived from an AAV9.

45. The vector of any one of claims 30-44, wherein the vector is optimized for expression in mammalian cells.

46. The vector of any one of claims 30-45, wherein the vector is optimized for expression in human cells.

47. A composition comprising the vector of any one of claims 30-46.

48. The composition of claim 47, further comprising a pharmaceutically acceptable carrier.

49. A cell comprising the composition of 45 or 46.

50. The cell of claim 49, wherein the cell is a human cell.

51. The cell of claim 49 or 50, wherein the cell is a cardiomyocyte.

52. The cell of claim 49 or 50, wherein the cell is an induced pluripotent stem (iPS) cell.

53. A composition comprising the cell of any one of claims 49-52.

54. A method for correcting a mutation in human RBM20, the method comprising contacting a cell with a composition of any one of claim 47 or 48 under conditions suitable for expression of the first gRNA and the adenine base editor, wherein the first gRNA forms a complex with the adenine base editor, wherein the complex modifies a mutation thereby restoring correct the coding sequence of RBM20.

55. A cell produced by the method of claim 54.

56. A method of treating dilated cardiomyopathy in a subject in need thereof, the method comprising administering to the subject a therapeutically effective amount of a composition of any one of claim 47 or 48.

57. The method of claim 56, wherein the composition is administered locally.

58. The method of claim 56 or 57, wherein the composition is administered directly to cardiac tissue.

59. The method of any one of claims 56-58, wherein the composition is administered by an infusion or injection.

60. The method of claim 56, wherein the composition is administered systemically.

61. The method of claim 60, wherein the composition is administered by an intravenous infusion or injection.

62. The method of any one of claims 56-61, wherein, following administration of the composition, the subject exhibits normal architecture of sarcomeric structures, nuclear localization of RBM20, absence of RNP granule formation, or a combination thereof.

63. The method of any one of claims 56-62, wherein, following administration of the composition, the subject exhibits improved LV function.

64. The method of any one of claims 56-63, wherein the subject is a neonate, an infant, a child, a young adult, or an adult.

65. The method of any one of claims 56-64, wherein the subject is male.

66. The method of any one of claims 56-64, wherein the subject is female.

67. Use of a therapeutically effective amount of a composition of any one of claim 47 or 48 for treating dilated cardiomyopathy in a subject in need thereof.

68. A guide RNA (gRNA) comprising a targeting nucleic acid sequence of 5′-GATATGGCCCAGAAAGGCCG-3′ (SEQ ID NO: 5).

69. The gRNA of claim 68, wherein the gRNA is a prime editing (pe) gRNA (pegRNA).

70. The gRNA of claim 68 or 69, wherein the gRNA is for modifying the human RBM20 gene to correct a C1906A mutation.

71. The gRNA of claim 68, wherein the gRNA further comprises a primer binding site comprising a nucleic acid sequence of 5′-CCTTTCTGGGC-3′ (SEQ ID NO: 6).

72. The gRNA of claim 71, wherein the gRNA further comprises a reverse transcriptase template comprising a nucleic acid sequence of 5′-GGACTACGAGAGCGCGG-3′ (SEQ ID NO: 7).

73. A composition comprising a gRNA that targets a mutation in human RBM20 and a prime editor.

74. The composition of claim 73, wherein the prime editor comprises a CRISPR/Cas nuclease linked to a reverse transcriptase.

75. The composition of claim 73, wherein the gRNA is the gRNA of any one of claims 89-94.

76. The composition of claim 75, wherein the prime editor comprises a CRISPR/Cas nuclease linked to a reverse transcriptase.

77. The composition of claim 76, wherein the CRISPR/Cas nuclease is catalytically impaired.

78. The composition of claim 76 or 77, wherein the CRISPR/Cas nuclease is a Cas9 nuclease.

79. The composition of claim 78, wherein the Cas9 nuclease is isolated or derived from Streptococcus pyogenes (spCas9).

80. The composition of any one of claims 73-79, further comprising a second-strand nicking sgRNA.

81. A nucleic acid comprising:
a sequence encoding a first gRNA of any one of claims 68-72,
a sequence encoding a prime editor,
a sequence encoding a first promoter, wherein the first promoter drives expression of the sequence encoding the first gRNA, and
a sequence encoding a second promoter, wherein the second promoter drives expression of the sequence encoding the prime editor.

82. The nucleic acid of claim 81, wherein the prime editor comprises a CRISPR/Cas nuclease linked to a reverse transcriptase.

83. The nucleic acid of claim 82, wherein the CRISPR/Cas nuclease is catalytically impaired.

84. The nucleic acid of claim 82 or 83, wherein the CRISPR/Cas nuclease is a Cas9 nuclease.

85. The nucleic acid of claim 84, wherein the Cas9 nuclease is isolated or derived from Streptococcus pyogenes (spCas9).

86. The nucleic acid of any one of claims 81-85, further comprising a sequence encoding a second-strand nicking sgRNA.

87. The nucleic acid any one of claims 81-86, wherein at least one of the sequence encoding the first promoter and the sequence encoding the second promoter comprises a cell-type specific promoter.

88. The nucleic acid of claim 87, wherein the cell-type specific promoter is a cardiomyocyte-specific promoter.

89. The nucleic acid of claim 88, wherein the muscle-specific promoter is a cardiac troponin T (cTnT) promoter.

90. The nucleic acid of any one of claims 81-89, wherein the sequence encoding the first promoter comprises a sequence encoding a U6 promoter, an H1 promoter, or a 7SK promoter.

91. The nucleic acid of any one of claims 81-90, wherein the nucleic acid comprises a DNA sequence.

92. The nucleic acid of any one of claims 81-91, wherein the nucleic acid comprises an RNA sequence.

93. The nucleic acid of any one of claims 81-92, wherein the nucleic acid further comprises a polyadenosine (polyA) sequence.

94. The nucleic acid of claim 93, wherein the polyA sequence is a mini polyA sequence.

95. A cell comprising the nucleic acid of any one of claims 81-94.

96. A composition comprising the nucleic acid of any one of claims 81-94.

97. A cell comprising the composition of claim 96.

98. A composition comprising the cell of claim 97.

99. A vector comprising the nucleic acid of any one of claims 81-94.

100. The vector of claim 99, wherein the vector further comprises a sequence encoding an inverted terminal repeat (ITR) of a transposable element.

101. The vector of claim 100, wherein the transposable element is a transposon.

102. The vector of claim 101, wherein the transposon is a Tn7 transposon.

103. The vector of claim 102, wherein the vector further comprises a sequence encoding a 5′ ITR of a T7 transposon and a sequence encoding a 3′ ITR of a T7 transposon.

104. The vector of any one of claims 99-103, wherein the vector is a non-viral vector.

105. The vector of claim 127, wherein the non-viral vector is a plasmid.

106. The vector of any one of claims 99-103, wherein the vector is a viral vector.

107. The vector of claim 129, wherein the viral vector is an adeno-associated viral (AAV) vector.

108. The vector of claim 107, wherein the AAV vector is replication-defective or conditionally replication defective.

109. The vector of claim 107 or 108, wherein the AAV vector is a recombinant AAV vector.

110. The vector of any one of claims 107-109, wherein the AAV vector comprises a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11) or any combination thereof.

111. The vector of any one of claims 107-110, wherein the AAV vector comprises a sequence isolated or derived from an AAV vector of serotype 9 (AAV9).

112. The vector of any one of claims 107-111, wherein the AAV vector comprises a sequence isolated or derived from an AAV vector of serotype 2 (AAV2).

113. The vector of any one of claims 107-112, wherein the AAV vector comprises a sequence isolated or derived from an AAV2 and a sequence isolated or derived from an AAV9.

114. The vector of any one of claims 99-113, wherein the vector is optimized for expression in mammalian cells.

115. The vector of any one of claims 99-114, wherein the vector is optimized for expression in human cells.

116. A composition comprising the vector of any one of claims 99-115.

117. The composition of claim 116, further comprising a pharmaceutically acceptable carrier.

118. A cell comprising the composition of 116 or 117.

119. The cell of claim 118, wherein the cell is a human cell.

120. The cell of claim 18 or 119, wherein the cell is a cardiomyocyte.

121. The cell of claim 118 or 119, wherein the cell is an induced pluripotent stem (iPS) cell.

122. A composition comprising the cell of any one of claims 118-121.

123. A method for correcting a mutation in human RBM20, the method comprising contacting a cell with a composition of any one of claim 116 or 117 under conditions suitable for expression of the first gRNA and the prime editor, wherein the first gRNA forms a complex with the prime editor, wherein the complex modifies a mutation thereby restoring correct the coding sequence of RBM20.

124. A cell produced by the method of claim 123.

125. A method of treating dilated cardiomyopathy in a subject in need thereof, the method comprising administering to the subject a therapeutically effective amount of a composition of any one of claim 116 or 117.

126. The method of claim 125, wherein the composition is administered locally.

127. The method of claim 125 or 126, wherein the composition is administered directly to a cardiac tissue.

128. The method of any one of claims 125-127, wherein the composition is administered by an infusion or injection.

129. The method of claim 125, wherein the composition is administered systemically.

130. The method of claim 129, wherein the composition is administered by an intravenous infusion or injection.

131. The method of any one of claims 125-130, wherein, following administration of the composition, the subject exhibits normal architecture of sarcomeric structures, nuclear localization of RBM20, absence of RNP granule formation, or a combination thereof.

132. The method of any one of claims 125-131, wherein, following administration of the composition, the subject exhibits improved LV function.

133. The method of any one of claims 125-132, wherein the subject is a neonate, an infant, a child, a young adult, or an adult.

134. The method of any one of claims 125-133, wherein the subject is male.

135. The method of any one of claims 125-133, wherein the subject is female.

136. Use of a therapeutically effective amount of a composition of any one of claim 116 or 117 for treating muscular dystrophy in a subject in need thereof.

137. A mouse whose genome comprises at least one allele of a Rbm20 encoded an R636Q mutation.

138. The mouse of claim 137, wherein the mouse has a C57/BL6 genetic background.

139. The mouse of claim 137, wherein the genome is homozygous for alleles of Rbm20 encoded an R636Q mutation.

140. The mouse of claim 137, wherein the mouse suffers from cardiac dysfunction.

141. The mouse of claim 140, wherein the left ventricular internal dimensions during end-diastole (LVIDd) and end-systole (LVIDs) are increased.

142. The mouse of claim 140, wherein the cardiac dysfunction is atrial and ventricular dilation.

143. The mouse of claim 140, wherein the cardiac dysfunction is reduced fractional shortening.

144. A cell isolated from a mouse of any one of claims 137-143.

145. The cell of claim 144, wherein the cell is a cardiomyocyte.

146. A method for screening at least one candidate agent in the mouse according to any one of claims 137-143, comprising administering one or more candidate agent to the mouse.

147. The method of claim 146, wherein the at least one candidate agent is screened for its ability to improve left ventricular function.

148. The method of claim 146, wherein the at least one candidate agent is screened for its ability to rescue cardiac chamber size.

149. The method of any one of claims 146-148, wherein the at least one candidate agent is screened for its ability to increase life span.

150. The method of any one of claims 146-148, wherein the candidate agent is a comprising the nucleic acid of any one of claims 12-25.
